(19) United States
(12) Patent Application Publication
(10) Pub. No.: US 2009/0270442 A1
(43) Pub. Date: Oct. 29, 2009

(54) POLYMORPHS OF HYDROCHLORIDE SALT OF 5-(3-(ETHYLSULFONYL)PHENYL)-3,8-DIMETHYL-N-(1-METHYLPIPERIDIN-4-YL)-9H-PYRIDO[2,3-B]INDOLE-7-CARBOXAMIDE AND METHODS OF USE THEREFOR

(75) Inventors: Lauren Graham, Altamont, NY (US); Paul Isbester, Castleton-on-Hudson, NY (US); Olga V. Lapina, Clifton Park, NY (US); Bingidimi I. Mobele, Altamont, NY (US); Grant J. Palmer, Clifton Park, NY (US); Karl Reineke, Niskayuna, NY (US); Jonathon S. Salsbury, Madison, WI (US); Luckner Ulysse, Albany, NY (US)

Correspondence Address: TAKEDA SAN DIEGO, INC., 10410 SCIENCE CENTER DRIVE, SAN DIEGO, CA, 92121, US

(73) Assignee: TAKEDA PHARMACEUTICAL COMPANY LIMITED, Osaka (JP)

(21) Appl. No.: 12/422,877

(22) Filed: Apr. 13, 2009

Related U.S. Application Data
(60) Provisional application No. 61/045,523, filed on Apr. 16, 2008.

(51) Int. Cl. A61K 31/437; C07D 471/04; A61P 35/00; A61P 25/28; A61P 25/00; A61P 19/00

(52) U.S. Cl. 514/292; 546/87

(57) ABSTRACT

Polymorphic forms of the hydrochloride salt of 5-(3-(ethylsulfonyl)phenyl)-3,8-dimethyl-N-(1-methylpiperidin-4-yl)-9h-pyrido[2,3-b]indole-7-carboxamide (referred to herein as Compound 1) which has the formula:

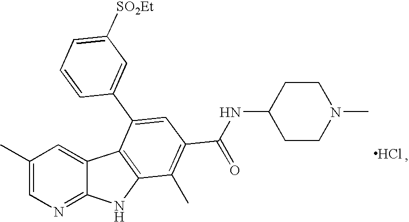

and compositions thereof, wherein the Compound 1 is present in one or more polymorphic forms. Also provided are novel methods for the preparation of the polymorphs of Compound 1, and kits and articles of manufacture of the compositions, and methods of using the compositions to treat various diseases.

### RELATED APPLICATION

This application claims benefit of U.S. Provisional Application No. 61/045,523 filed on Apr. 16, 2008, which is incorporated herein by reference.

### FIELD OF THE INVENTION

The present invention relates generally to polymorphic forms of the hydrochloric acid salt of 5-(3-(ethylsulfonyl)phenyl)-3,8-dimethyl-N-(1-methylpiperidin-4-yl)-9H-pyrido[2,3-b]indole-7-carboxamide, (referred to herein as “Compound 1”) and methods for their preparation. The present invention also relates to pharmaceutical compositions, kits and articles of manufacture comprising polymorphs of Compound 1, and methods of their use.

### DESCRIPTION OF RELATED ART

Compound 1, which has the formula:

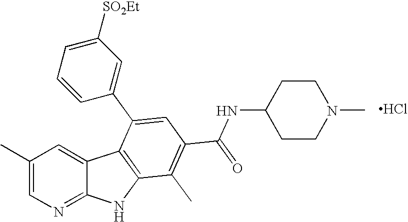

is a kinase inhibitor that is described in U.S. Patent Publication No. 2007-0117816, published May 24, 2007 (see Compound 112) and U.S. Patent Application Nos. 60/912,625 and 60/912,629, filed Apr. 18, 2007 (see Compound 83), which are incorporated herein by reference in their entireties.

Phosphoryl transferases are a large family of enzymes that transfer phosphorous-containing groups from one substrate to another. By the conventions set forth by the Nomenclature Committee of the International Union of Biochemistry and Molecular Biology (IUBMB) enzymes of this type have Enzyme Commission (EC) numbers starting with 2.7 . . . . (See, Bairoch A., The ENZYME database in Nucleic Acids Res. 28:204-305 (2000)). Kinases are a class of enzymes that function in the catalysis of phosphoryl transfer. The protein kinases constitute the largest subfamily of structurally related phosphoryl transferases and are responsible for the control of a wide variety of signal transduction processes within the cell. (See, Hardie, G. and Hanks, S. (1995) The Protein Kinase Facts Book, I and II, Academic Press, San Diego, Calif.). Protein kinases are thought to have evolved from a common ancestral gene due to the conservation of their structure and catalytic function. Almost all kinases contain a similar 250-300 amino acid catalytic domain. The protein kinases may be categorized into families by the substrates they phosphorylate (e.g., protein-tyrosine, protein-serine/threonine, histidine, etc.). Protein kinase sequence motifs have been identified that generally correspond to each of these kinase families (See, for example, Hanks, S. K.; Hunter, T., FASEB J. 9:576-596 (1995); Kinghton et al., Science, 253:407-414 (1991); Hiles et al., Cell 70:419-429 (1992); Kunz et al., Cell, 73:585-596 (1993); Garcia-Bustos et al., EMBO J., 13:2352-2361 (1994)). Lipid kinases (e.g. PI3K) constitute a separate group of kinases with structural similarity to protein kinases.

Protein and lipid kinases regulate many different cell processes including, but not limited to, proliferation, growth, differentiation, metabolism, cell cycle events, apoptosis, motility, transcription, translation and other signaling processes, by adding phosphate groups to targets such as proteins or lipids. Phosphorylation events catalyzed by kinases act as molecular on/off switches that can modulate or regulate the biological function of the target protein. Phosphorylation of target proteins occurs in response to a variety of extracellular signals (hormones, neurotransmitters, growth and differentiation factors, etc.), cell cycle events, environmental or nutritional stresses, etc. Protein and lipid kinases can function in signaling pathways to activate or inactivate, or modulate the activity of (either directly or indirectly) the targets. These targets may include, for example, metabolic enzymes, regulatory proteins, receptors, cytoskeletal proteins, ion channels or pumps, or transcription factors. Uncontrolled signaling due to defective control of protein phosphorylation has been implicated in a number of diseases and disease conditions, including, for example, inflammation, cancer, allergy/asthma, diseases and conditions of the immune system, disease and conditions of the central nervous system (CNS), cardiovascular disease, dermatology, and angiogenesis.

Initial interest in protein kinases as pharmacological targets was stimulated by the findings that many viral oncogenes encode structurally modified cellular protein kinases with constitutive enzyme activity. These findings pointed to the potential involvement of oncogene related protein kinases in human proliferatives disorders. Subsequently, deregulated protein kinase activity, resulting from a variety of more subtle mechanisms, has been implicated in the pathophysiology of a number of important human disorders including, for example, cancer, CNS conditions, and immunologically related diseases. The development of selective protein kinase inhibitors that can block the disease pathologies and/or symptoms resulting from aberrant protein kinase activity has therefore generated much interest.

Cancer results from the deregulation of the normal processes that control cell division, differentiation and apoptotic cell death. Protein kinases play a critical role in this regulatory process. A partial non-limiting list of such kinases includes abl, Aurora-A, Aurora-B, Aurora-C, ATK, bcr-abl, Blk, Brk, Btk, c-Kit, c-Met, c-Src, CDK1, CDK2, CDK4, CDK6, cRaf1, CSF1R, CSK, EGFR, ErbB2, ErbB3, ErbB4, ERK, Fak, fes, FGFR1, FGFR2, FGFR3, FGFR4, FGFR5, Fgr, FLK-4, Flt-1, Fps, Frk, Fyn, Hck, IGF-1R, INS-R, Jak, KDR, Lck, Lyn, MEK, p38, PDGFR, PIK, PKC, PYK2, Ros, Tie1, Tie2, Trk, Yes and Zap70. In mammalian biology, such protein kinases comprise mitogen activated protein kinase (MAPK) signaling pathways. MAPK signaling pathways are inappropriately activated by a variety of common disease-associated mechanisms such as mutation of ras genes and deregulation of growth factor receptors (Magnuson et al., Seminars in Cancer Biology 5:247-252 (1994)). Therefore the inhibition of protein kinases is an object of the present invention.

Aurora kinases (Aurora-A, Aurora-B, Aurora-C) are serine/threonine protein kinases that have been implicated in human cancer, such as colon, breast and other solid tumors. Aurora-A (also sometimes referred to as AIK) is believed to be involved in protein phosphorylation events that regulate the cell cycle. Specifically, Aurora-A may play a role in controlling the accurate segregation of chromosomes during mitosis. Misregulation of the cell cycle can lead to cellular proliferation and other abnormalities. In human colon cancer tissue, Aurora-A, Aurora-B and Aurora-C have been found to be overexpressed (See, Bischoff et al., EMBO J., 17:3052-3065 (1998); Schumacher et al., J. Cell Biol. 143:1635-1646 (1998); Kimura et al., J. Biol. Chem., 272:13766-13771 (1997)).

Kinase inhibitors are believed to be useful agents for the prevention, delay of progression, and/or treatment of conditions mediated by kinases.

### SUMMARY OF THE INVENTION

The present invention provides novel polymorphic forms of Compound 1 and methods of preparing these polymorphic forms, as well as compositions comprising one or more of the novel polymorphs.

### Polymorphic Forms

In one aspect, the invention provides polymorphic forms of Compound 1 having the formula:

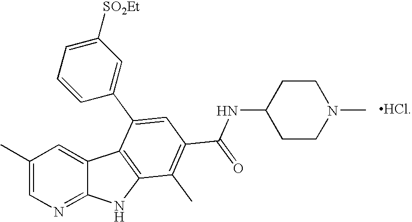

Various methods are also provided for making Amorphous Form, Form A, Form B, Form C, Form D, Form E, Form F, Form G, Form I, Form J, Form K, Form L, Form M, Form N, Form O, and Form P. Various methods are also provided for manufacturing pharmaceutical compositions, kits and other articles of manufacture comprising one or more of Amorphous Form, Form A, Form B, Form C, Form D, Form E, Form F, Form G, Form I, Form J, Form K, Form L, Form M, Form N, Form O and Form P.

### Amorphous Form:

In one embodiment, the polymorphic form is an amorphous solid having an X-ray powder diffraction pattern (CuKα) comprising a broad diffraction peak at about 25.5 degrees 2-theta (°2θ). In some variations, the X-ray diffraction pattern is substantially as shown in FIG. 1.

### Form A:

In another embodiment, the polymorphic form is a monohydrate having an X-ray powder diffraction pattern (CuKα) comprising significant diffraction peaks at about 5.2, 10.3 and 20.5 degrees 2-theta (°2θ). In some variations, the X-ray powder diffraction pattern further comprises significant diffraction peaks at about 15.5, 17.0 and 19.9°2θ. In other variations, the X-ray diffraction pattern is substantially as shown in FIG. 2.

In yet another embodiment, the polymorphic form is a monohydrate having a differential scanning calorimetry (DSC) curve comprising an endotherm centered from about 315° C. to about 330° C. In some variations the endotherm is centered at about 327° C. In further variations, the DSC curve is substantially as shown in FIG. 3.

### Form B:

In still another embodiment, the polymorphic form is a dimethylacetamide (DMA) solvate having an X-ray powder diffraction pattern (CuKα) comprising significant diffraction peaks at about 13.8, 17.1 and 19.7°2θ. In some variations, the X-ray powder diffraction pattern further comprises significant diffraction peaks at about 16.5, 20.1 and 25.0°2θ. In further variations the X-ray diffraction pattern is substantially as shown in FIG. 7.

In a further embodiment, the polymorphic form is a dimethylacetamide (DMA) solvate having a differential scanning calorimetry (DSC) curve comprising an endotherm centered from about 330° C. to about 340° C. In some variations the endotherm is centered at about 337° C. In other variations the polymorphic form has substantially a DSC curve as shown in FIG. 8.

### Form C:

In a still further embodiment, the polymorphic form is an anhydrate having an X-ray powder diffraction pattern (CuKα) comprising significant diffraction peaks at about 17.1, 19.8 and 26.4°2θ. In some variations the X-ray powder diffraction pattern further comprises significant diffraction peaks at about 17.7 and 22.0°2θ. In other variations, the X-ray diffraction pattern is substantially as shown in FIG. 11.

In another embodiment, the polymorphic form is an anhydrate having a differential scanning calorimetry (DSC) curve comprising an endotherm centered from about 332° C. to about 336° C. In some variations the endotherm is centered at about 335° C. In other variations the polymorphic form has a DSC curve substantially as shown in FIG. 12.

### Form D:

In still another embodiment, the polymorphic form is an anhydrate having an X-ray powder diffraction pattern (CuKα) comprising significant diffraction peaks at about 7.8, 17.6, and 20.9°2θ. In some variations the X-ray powder diffraction pattern further comprises significant diffraction peaks at about 5.9 and 25.2°2θ. In other variations the X-ray diffraction pattern is substantially as shown in FIG. 16.

In yet another embodiment, the polymorphic form is an anhydrate having a differential scanning calorimetry (DSC) curve comprising an endotherm centered from about 245° C. to about 255° C. In some variations, the endotherm is centered at about 251° C. In other variations, the polymorphic form has a DSC curve substantially as shown in FIG. 17.

### Form E:

In still yet another embodiment, the polymorphic form is a N-methylpyrrolidinone (NMP) solvate having an X-ray powder diffraction pattern (CuKα) comprising significant diffraction peaks at about 17.0, 19.6 and 20.2°2θ. In some variations the X-ray powder diffraction pattern further comprises significant diffraction peaks at about 13.9, 25.1 and 26.2 °2θ. In other variations, the X-ray diffraction pattern is substantially as shown in FIG. 20.

In a further embodiment, the polymorphic form is a N-methylpyrrolidinone (NMP) solvate having a differential scanning calorimetry (DSC) curve comprising an endotherm centered from about 215° C. to about 225° C. In some variations, the endotherm is centered at about 221° C. In other variations, the polymorphic form has a DSC curve substantially as shown in FIG. 21.

### Form F:

In yet a further embodiment, the polymorphic form is a desolvate having an X-ray powder diffraction pattern (CuKα) comprising significant diffraction peaks at about 7.0, 17.2, and 25.9°2θ. In some variations, the X-ray powder diffraction pattern further comprises significant diffraction peaks at about 5.2, 10.3 and 20.2°2θ. In other variations, the X-ray diffraction pattern is substantially as shown in FIG. 24.

In another embodiment, the polymorphic form is a desolvate having a differential scanning calorimetry (DSC) curve comprising an endotherm centered from about 323° C. to about 333° C. In some variations, the endotherm is centered at about 328° C. In other variations, the polymorphic form has a DSC curve substantially as shown in FIG. 25.

### Form G:

In still another embodiment, the polymorphic form is a dimethylformamide (DMF) solvate having an X-ray powder diffraction pattern (CuKα) comprising significant diffraction peaks at about 5.5, 10.9 and 22.0°2θ. In some variations, the X-ray powder diffraction pattern further comprises significant diffraction peaks at about 16.5, 18.4 and 19.5°2θ. In other variations, the X-ray diffraction pattern is substantially as shown in FIG. 27.

In yet another embodiment, the polymorphic form is a dimethylformamide (DMF) solvate having a differential scanning calorimetry (DSC) curve comprising an endotherm centered from about 334° C. to about 338° C. In some variations, the endotherm is centered at about 336° C. In some variations, the polymorphic form has a DSC curve substantially as shown in FIG. 28.

### Form I:

In still yet another embodiment, the polymorphic form is a tetrahydrofuran (THF) solvate having an X-ray powder diffraction pattern (CuKα) comprising significant diffraction peaks at about 7.0, 16.7 and 17.4°2θ. In some variations, the X-ray powder diffraction pattern further comprises significant diffraction peaks at about 19.6, 20.2 and 24.6°2θ. In other variations, the X-ray diffraction pattern is substantially as shown in FIG. 31.

In a further embodiment, the polymorphic form is a tetrahydrofuran (THF) solvate having a differential scanning calorimetry (DSC) curve comprising an endotherm centered from about 320° C. to about 340° C. In some variations the endotherm is centered at about 331° C. In other variations, the polymorphic form has substantially a DSC curve substantially as shown in FIG. 32.

### Form J:

In a still further embodiment, the polymorphic form is an anhydrate having an X-ray powder diffraction pattern (CuKα) comprising significant diffraction peaks at about 4.9, 17.5 and 20.0°2θ. In some variations the X-ray powder diffraction pattern (CuKα) further comprises significant diffraction peaks at about 9.2, 22.1 and 25.2°2θ. In other variations the X-ray diffraction pattern (CuKα) is substantially as shown in FIG. 35.

In another embodiment, the polymorphic form is an anhydrate having a differential scanning calorimetry (DSC) curve comprising a forked endotherm centered from about 320° C. to about 330° C. In some variations the forked endotherm is centered at about 326° C. In other variations, the polymorphic form has substantially a DSC curve substantially as shown in FIG. 36.

### Form K:

In still another embodiment, the polymorphic form is an anhydrate having an X-ray powder diffraction pattern (CuKα) comprising significant diffraction peaks at about 5.3, 8.5 and 10.5°2θ. In some variations, the X-ray powder diffraction pattern (CuKα) further comprises significant diffraction peaks at about 13.3, 18.6 and 21.3°2θ. In other variations, the X-ray diffraction pattern (CuKα) is substantially as shown in FIG. 39.

In yet another embodiment, the polymorphic form is an anhydrate having a differential scanning calorimetry (DSC) curve comprising an endotherm centered from about 315° C. to about 330° C. In some variations, the endotherm is centered at about 322° C. In other variations, the polymorphic form has a DSC curve substantially as shown in FIG. 40.

### Form L:

In a further embodiment, the polymorphic form is a channel hydrate having an X-ray powder diffraction pattern (CuKα) comprising significant diffraction peaks at about 5.2, 10.4 and 20.7°2θ. In some variations, the X-ray powder diffraction pattern (CuKα) further comprises significant diffraction peaks at about 15.5, 16.9 and 24.4°2θ. In other variations, the X-ray diffraction pattern (CuKα) is substantially as shown in FIG. 43.

In still a further embodiment, the polymorphic form is a channel hydrate having a differential scanning calorimetry (DSC) curve comprising an endotherm centered from about 320° C. to about 340° C. In some variations, the endotherm is centered at about 333° C. In other variations, the polymorphic form has a DSC curve substantially as shown in FIG. 44.

### Form M:

In another embodiment, the polymorphic form is a hydrate having an X-ray powder diffraction pattern (CuKα) comprising significant diffraction peaks at about 5.1, 8.2 and 10.2°2θ. In some variations the X-ray powder diffraction pattern (CuKα) further comprises significant diffraction peaks at about 18.1 and 20.6°2θ. In other variations the X-ray diffraction pattern (CuKα) is substantially as shown in FIG. 48.

In still another embodiment, the polymorphic form is a hydrate having a differential scanning calorimetry (DSC) curve comprising an endotherm centered from about 325° C. to about 335° C. In some variations, the endotherm is centered at about 332° C. In other variations, the polymorphic form has a DSC curve substantially as shown in FIG. 49.

### Form N:

In yet another embodiment, the polymorphic form is a hydrate having an X-ray powder diffraction pattern (CuKα) comprising significant diffraction peaks at about 5.2, 8.4 and 10.3°2θ. In some variations, the X-ray powder diffraction pattern (CuKα) further comprises significant diffraction peaks at about 18.6, 20.0 and 21.0°2θ. In other variations the X-ray diffraction pattern (CuKα) is substantially as shown in FIG. 52.

In a further embodiment, the polymorphic form is a hydrate having a differential scanning calorimetry (DSC) curve comprising an endotherm centered from about 326° C. to about 336° C. In some variations, the endotherm is centered at about 331° C. In other variations, the polymorphic form has a DSC curve substantially as shown in FIG. 53.

### Form O:

In a still further embodiment, the polymorphic form is a dehydrate having an X-ray powder diffraction pattern (CuKα) comprising significant diffraction peaks at about 6.3, 12.6 and 25.3°2θ. In some variations the X-ray powder diffraction pattern (CuKα) further comprises significant diffraction peaks at about 10.5 and 21.0°2θ. In other variations, the X-ray diffraction pattern (CuKα) is substantially as shown in FIG. 56.

In another embodiment, the polymorphic form is a dehydrate having a differential scanning calorimetry (DSC) curve comprising an endotherm centered from about 320° C. to about 330° C. In some variations, the endotherm is centered at about 327° C. In other variations, the polymorphic form has a DSC curve substantially as shown in FIG. 57.

### Form P:

In still another embodiment, the polymorphic form has an X-ray powder diffraction pattern (CuKα) comprising significant diffraction peaks at about 5.0, 9.4 and 10.0°2θ. In some variations, the X-ray powder diffraction pattern (CuKα) further comprises significant diffraction peaks at about 17.2 and 25.7°2θ. In other variations, the X-ray diffraction pattern (CuKα) is substantially as shown in FIG. 59.

### Methods of Making Polymorphic Forms

In another aspect, the invention provides methods of making polymorphic forms of Compound 1 having the formula:

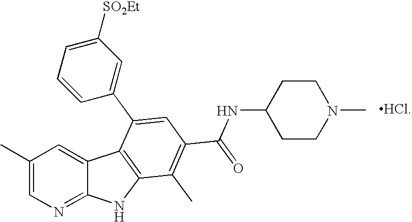

In one embodiment, the polymorphic form is Form A (e.g., a monohydrate having an X-ray powder diffraction pattern (CuKα) comprising significant diffraction peaks at about 5.2, 10.3 and 20.5°2θ), and the method comprises treating Compound 1 with water. In some variations, the method further comprises dissolving Compound 1 in DMF. In other variations, the method further comprises adding an antisolvent to Compound 1 dissolved in the solvent, wherein the antisolvent is isopropyl acetate.

In another embodiment, the polymorphic form is Form B (e.g., a dimethylacetamide (DMA) solvate having an X-ray powder diffraction pattern comprising significant diffraction peaks at about 13.8, 17.1 and 19.7°2θ), and the method comprises treating Compound 1 with DMA. In some variations, the method further comprises dissolving Compound 1 in DMA.

In a further embodiment, the polymorphic form is Form C (e.g., an anhydrate having an X-ray powder diffraction pattern (CuKα) comprising significant diffraction peaks at about 17.1, 19.8 and 26.4°2θ), and the method comprises drying Compound 1. In some variations, the method further comprises drying Compound 1 at a temperature above 50° C. In other variations, the method further comprises drying Compound 1 at a temperature above 70° C.

In still a further embodiment, the polymorphic form is Form C (e.g., an anhydrate having an X-ray powder diffraction pattern (CuKα) comprising significant diffraction peaks at about 17.1, 19.8 and 26.4°2θ), and the method comprises dissolving Compound 1 in an anhydrous solvent.

In another embodiment, the polymorphic form is Form D (e.g., an anhydrate having an X-ray powder diffraction pattern (CuKα) comprising significant diffraction peaks at about 7.8, 17.6, and 20.9°2θ), and the method comprises treating Compound 1 with DMA. In some variations, the method further comprises dissolving Compound 1 in DMA. In other variations, the method further comprises adding an antisolvent to Compound 1 dissolved in the solvent, wherein the antisolvent is methyl tert-butylether (MTBE).

In still another embodiment, the polymorphic form is Form E (e.g., a N-methyl pyrrolidinone (NMP) solvate having an X-ray powder diffraction pattern (CuKα) comprising significant diffraction peaks at about 17.0, 19.6 and 20.2°2θ), and the method comprises treating Compound 1 with NMP.

In yet another embodiment, the polymorphic form is Form F (e.g., a desolvate having an X-ray powder diffraction pattern (CuKα) comprising significant diffraction peaks at about 7.0, 17.2, and 25.9°2θ), and the method comprises treating Compound 1 with DMA or DMF. In some variations, the method further comprises heating Compound 1.

In another embodiment, the polymorphic form is Form G (e.g., a dimethylformamide (DMF) solvate having an X-ray powder diffraction pattern (CuKα) comprising significant diffraction peaks at about 5.5, 10.9 and 22.0°2θ), and the method comprises treating Compound 1 with DMF.

In still another embodiment, the polymorphic form is Form I (e.g., a tetrahydrofuran (THF) solvate having an X-ray powder diffraction pattern (CuKα) comprising significant diffraction peaks at about 7.0, 16.7 and 17.4°2θ), and the method comprises treating Compound 1 with THF.

In another embodiment, the polymorphic form is Form J (e.g., an anhydrate having an X-ray powder diffraction pattern (CuKα) comprising significant diffraction peaks at about 4.9, 17.5 and 20.0°2θ), and the method comprises treating Compound 1 with isopropyl alcohol.

In still another embodiment, the polymorphic form is Form K (e.g., an anhydrate having an X-ray powder diffraction pattern (CuKα) comprising significant diffraction peaks at about 5.3, 8.5 and 10.5°2θ), and the method comprises treating Compound 1 with THF. In some variations, the method further comprises dissolving Compound 1 in EtOH. In other variations, the method further comprises adding an antisolvent to Compound 1 dissolved in the solvent, wherein the antisolvent is THF.

In yet another embodiment, the polymorphic form is Form L (e.g., a channel hydrate having an X-ray powder diffraction pattern (CuKα) comprising significant diffraction peaks at about 5.2, 10.4 and 20.7°2θ), and the method comprises treating Compound 1 with water. In some variations, the method further comprises dissolving Compound 1 in methanol. In other variations, the method further comprises adding an antisolvent to Compound 1 dissolved in the solvent, wherein the antisolvent is selected from the group consisting of methyl tert-butylether, isopropyl acetate and heptane.

In a further embodiment, the polymorphic form is Form M (e.g., a hydrate having an X-ray powder diffraction pattern (CuKα) comprising significant diffraction peaks at about 5.1, 8.2 and 10.2°2θ), and the method comprises treating Compound 1 with water.

In a still further embodiment, the polymorphic form is Form N (e.g., a hydrate having an X-ray powder diffraction pattern (CuKα) comprising significant diffraction peaks at about 5.2, 8.4 and 10.3°2θ), and the method comprises treating Compound 1 with water.

In another embodiment, the polymorphic form is Form O (e.g., a dehydrate having an X-ray powder diffraction pattern (CuKα) comprising significant diffraction peaks at about 6.3, 12.6 and 25.3°2θ), and the method comprises treating Compound 1 with water. In some variations, the method further comprises heating Compound 1.

Methods by which the above referenced analyses were performed in order to identify these physical characteristics are described in the Examples section below.

### Compositions Comprising Compound 1

In a further aspect, the invention provides pharmaceutical compositions comprising Compound 1 of the formula:

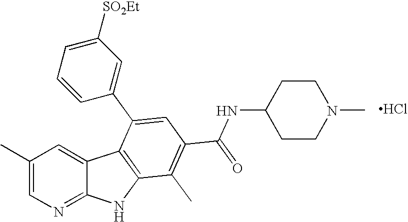

wherein at least a portion of Compound 1 is present as a polymorphic form, such as any polymorphic form described throughout this application.

In some embodiments, Compound 1 is present in a form selected from the group consisting of Amorphous Form, Form A, Form B, Form C, Form D, Form E, Form F, Form G, Form I, Form J, Form K, Form L, Form M, Form N, Form O and/or Form P. These forms are described in greater detail below. It is noted that other crystalline and amorphous forms of Compound 1 may also be present in the composition.

In one variation, the composition comprises at least 0.1%, 0.25%, 0.5%, 1%, 5%, 10%, 25%, 50%, 75%, 80%, 85%, 90%, 95%, 97%, or 99% of Compound 1 where greater than 0.1%, 0.25%, 0.5%, 1%, 5%, 10%, 25%, 50%, 75%, 80%, 85%, 90%, 95%, 97% or 99% of Compound 1 (by weight) is present in the composition in a form selected from the group consisting of Amorphous Form, Form A, Form B, Form C, Form D, Form E, Form F, Form G, Form I, Form J, Form K, Form L, Form M, Form N, Form O and Form P. The composition may optionally be a pharmaceutical composition. The pharmaceutical composition may optionally further include one or more additional components that do not deleteriously affect the use of Compound 1.

### Kits and Articles of Manufacture Comprising Compound 1

The invention also provides kits and other articles of manufacture comprising a composition that comprises Compound 1, wherein Compound 1 is present in a form selected from the group consisting of Amorphous Form, Form A, Form B, Form C, Form D, Form E, Form F, Form G, Form I, Form J, Form K, Form L, Form M, Form N, Form O and Form P. In one variation, the composition comprises at least 0.1%, 0.25%, 0.5%, 1%, 5%, 10%, 25%, 50%, 75%, 80%, 85%, 90%, 95%, 97%, or 99% of Compound 1 where greater than 0.1%, 0.25%, 0.5%, 1%, 5%, 10%, 25%, 50%, 75%, 80%, 85%, 90%, 95%, 97% or 99% of Compound 1 (by weight) is present in the composition in a form selected from the group consisting of Amorphous Form, Form A, Form B, Form C, Form D, Form E, Form F, Form G, Form I, Form J, Form K, Form L, Form M, Form N, Form O and Form P. The composition in the kits and articles of manufacture may optionally be a pharmaceutical composition. The pharmaceutical composition may optionally further include one or more additional components that do not deleteriously affect the use of Compound 1.

In regard to each of the above embodiments including a pharmaceutical composition, the pharmaceutical composition may be formulated in any manner where at least a portion of Compound 1 is present in a form selected from the group consisting of Amorphous Form, Form A, Form B, Form C, Form D, Form E, Form F, Form G, Form I, Form J, Form K, Form L, Form M, Form N, Form O and Form P. Optionally, a portion of Compound 1 is present in a form selected from the group consisting of Amorphous Form, Form A, Form B, Form C, Form D, Form E, Form F, Form G, Form I, Form J, Form K, Form L, Form M, Form N, Form O and Form P for a period of time subsequent to administration of the pharmaceutical formulation to a subject.

### Methods of Using Polymorphic Forms

Methods of using a pharmaceutical composition, kit and other article of manufacture comprising one or more of Amorphous Form, Form A, Form B, Form C, Form D, Form E, Form F, Form G, Form I, Form J, Form K, Form L, Form M, Form N, Form O and Form P to treat various diseases mediated by a kinase are also provided.

In one embodiment, the present invention relates to a method of inhibiting kinases comprising administering a composition where greater than 0.1%, 0.25%, 0.5%, 1%, 5%, 10%, 25%, 50%, 75%, 80%, 85%, 90%, 95%, 97% or 99% of Compound 1 (by weight) is present in the composition in a form selected from the group consisting of Amorphous Form, Form A, Form B, Form C, Form D, Form E, Form F, Form G, Form I, Form J, Form K, Form L, Form M, Form N, Form O and Form P. Optionally, the composition comprises at least 0.1%, 0.25%, 0.5%, 1%, 5%, 10%, 25%, 50%, 75%, 80%, 85%, 90%, 95%, 97%, or 99% of Compound 1.

In another embodiment, the present invention relates to a method of inhibiting kinases in a subject (e.g., human body) with Compound 1 by administering Compound 1 where greater than 0.1%, 0.25%, 0.5%, 1%, 5%, 10%, 25%, 50%, 75%, 80%, 85%, 90%, 95%, 97% or 99% of Compound 1 (by weight) is present in the composition in a form selected from the group consisting of Amorphous Form, Form A, Form B, Form C, Form D, Form E, Form F, Form G, Form I, Form J, Form K, Form L, Form M, Form N, Form O and Form P, when the compound is administered. Optionally, the composition comprises at least 0.1%, 0.25%, 0.5%, 1%, 5%, 10%, 25%, 50%, 75%, 80%, 85%, 90%, 95%, 97%, or 99% of Compound 1.

In another embodiment, the present invention relates to a method of inhibiting kinases in a subject (e.g., human body) with Compound 1 by administering Compound 1 where greater than 0.1%, 0.25%, 0.5%, 1%, 5%, 10%, 25%, 50%, 75%, 80%, 85%, 90%, 95%, 97% or 99% of Compound 1 (by weight) is present in the composition in a form selected from the group consisting of Amorphous Form, Form A, Form B, Form C, Form D, Form E, Form F, Form G, Form I, Form J, Form K, Form L, Form M, Form N, Form O and Form P for a period of time after the compound has been administered to a subject. Optionally, the composition comprises at least 0.1%, 0.25%, 0.5%, 1%, 5%, 10%, 25%, 50%, 75%, 80%, 85%, 90%, 95%, 97%, or 99% of Compound 1.

In still another embodiment, the present invention provides a method of treating a disease state for which kinases possess activity that contributes to the pathology and/or symptomology of the disease state, comprising administering to a subject (e.g., human body) a composition where greater than 0.1%, 0.25%, 0.5%, 1%, 5%, 10%, 25%, 50%, 75%, 80%, 85%, 90%, 95%, 97% or 99% of Compound 1 (by weight) is present in the composition in a form selected from the group consisting of Amorphous Form, Form A, Form B, Form C, Form D, Form E, Form F, Form G, Form I, Form J, Form K, Form L, Form M, Form N, Form O and Form P when administered. Optionally, the composition comprises at least 0.1%, 0.25%, 0.5%, 1%, 5%, 10%, 25%, 50%, 75%, 80%, 85%, 90%, 95%, 97%, or 99% of Compound 1.

In still another embodiment, the present invention provides a method of treating a disease state for which kinases possess activity that contributes to the pathology and/or symptomology of the disease state, comprising causing a composition to be present in a subject (e.g., human body) where greater than 0.1%, 0.25%, 0.5%, 1%, 5%, 10%, 25%, 50%, 75%, 80%, 85%, 90%, 95%, 97% or 99% of Compound 1 (by weight) is present in the composition in a form selected from the group consisting of Amorphous Form, Form A, Form B, Form C, Form D, Form E, Form F, Form G, Form I, Form J, Form K, Form L, Form M, Form N, Form O and Form P, for a period of time after the composition has been administered to a subject. Optionally, the composition comprises at least 0.1%, 0.25%, 0.5%, 1%, 5%, 10%, 25%, 50%, 75%, 80%, 85%, 90%, 95%, 97%, or 99% of Compound 1.

In another embodiment, a method is provided for preventing, delaying the progression of, and/or treating conditions mediated by kinases, in particular cancer (e.g., squamous cell carcinoma, astrocytoma, Kaposi's sarcoma, glioblastoma, small-cell lung cancer, non small-cell lung cancers (e.g., large cell lung cancer, adenocarcinoma and squamous cell carcinoma), bladder cancer, head and neck cancer, melanoma, ovarian cancer, prostate cancer, breast cancer, glioma, colorectal cancer, genitourinary cancer, gastrointestinal cancer, thyroid cancer, skin cancer and blood cancers (e.g., multiple myeloma, chronic myelogenous leukemia and acute lymphocytic leukemia)); inflammation; inflammatory bowel disease; psoriasis; transplant rejection; amyotrophic lateral sclerosis; corticobasal degeneration; Down syndrome; Huntington's Disease; Parkinson's Disease; postencephelatic parkinsonism; progressive supranuclear palsy; Pick's Disease; Niemann-Pick's Disease; stroke; head trauma; chronic neurodegenerative diseases; Bipolar Disease; affective disorders; depression; schizophrenia; cognitive disorders; hair loss; contraceptive medication; mild Cognitive Impairment; Age-Associated Memory Impairment; Age-Related Cognitive Decline; Cognitive Impairment No Dementia; mild cognitive decline; mild neurocognitive decline; Late-Life Forgetfulness; memory impairment; cognitive impairment; androgenetic alopecia; dementia related diseases (e.g., Frontotemporal dementia Parkinson's Type, Parkinson dementia complex of Guam, HIV dementia, diseases with associated neurofibrillar tangle pathologies, predemented states, vascular dementia, dementia with Lewy bodies, Frontotemporal dementia and dementia pugilistica); Alzheimer's Disease; arthritis; and others.

In each instance where it is stated that Compound 1 may be present in the composition in a form selected from the group consisting of Amorphous Form, Form A, Form B, Form C, Form D, Form E, Form F, Form G, Form I, Form J, Form K, Form L, Form M, Form N, Form 0 and Form P, it is intended for the invention to encompass compositions where only one form is present, where two forms are present (all combinations) and where three, four or more forms are present (all combinations).

### BRIEF DESCRIPTION OF THE FIGURES

FIG. 1 is a X-ray powder diffraction (XRPD) spectrum of Amorphous Form of Compound 1.

FIG. 2 is an XRPD pattern of Form A of Compound 1.

FIG. 3 is a differential scanning calorimetry (DSC) curve of Form A of Compound 1.

FIG. 4 is a thermal gravimetric analysis (TGA) curve of Form A of Compound 1.

FIG. 5 is an ¹H NMR spectrum of Form A of Compound 1.

FIG. 6 is a moisture sorption curve of Form A of Compound 1.

FIG. 7 is an XRPD pattern of Form B of Compound 1.

FIG. 8 is a DSC curve of Form B of Compound 1.

FIG. 9 is a TGA curve of Form B of Compound 1.

FIG. 10 is an ¹H NMR spectrum of Form B of Compound 1.

FIG. 11 is an XRPD pattern of Form C of Compound 1.

FIG. 12 is a DSC curve of Form C of Compound 1.

FIG. 13 is a TGA curve of Form C of Compound 1.

FIG. 14 is a ¹H NMR spectrum of Form C of Compound 1.

FIG. 15 is a moisture sorption curve of Compound 1.

FIG. 16 is an XRPD pattern of Form D of Compound 1.

FIG. 17 is a DSC curve of Form D of Compound 1.

FIG. 18 is a TGA curve of Form D of Compound 1.

FIG. 19 is an is a ¹H NMR spectrum of Form D of Compound 1.

FIG. 20 is a XRPD pattern of Form E of Compound 1.

FIG. 21 is a DSC curve of Form E of Compound 1.

FIG. 22 is a TGA curve of Form E of Compound 1.

FIG. 23 is a ¹H NMR spectrum of Form E of Compound 1.

FIG. 24 is a XRPD pattern of Form F of Compound 1.

FIG. 25 is a DSC curve of Form F of Compound 1.

FIG. 26 is a ¹H NMR spectrum of Form F of Compound 1.

FIG. 27 is a XRPD pattern of Form G of Compound 1.

FIG. 28 is a DSC curve of Form G of Compound 1.

FIG. 29 is a TGA curve of Form G of Compound 1.

FIG. 30 is a ¹H NMR spectrum of Form G of Compound 1.

FIG. 31 is a XRPD pattern of Form I of Compound 1.

FIG. 32 is a DSC curve Form I of Compound 1.

FIG. 33 is a TGA curve of Form I of Compound 1.

FIG. 34 is a ¹H NMR spectrum of Form I of Compound 1.

FIG. 35 is an XRPD pattern of Form J of Compound 1.

FIG. 36 is a DSC curve of Form J of Compound 1.

FIG. 37 is a TGA curve of Form J of Compound 1.

FIG. 38 is a ¹H NMR spectrum of Form J of Compound 1.

FIG. 39 is an XRPD pattern of Form K of Compound 1.

FIG. 40 is a DSC curve of Form K of Compound 1.

FIG. 41 is a TGA curve of Form K of Compound 1.

FIG. 42 is a ¹H NMR spectrum of Form K of Compound 1.

FIG. 43 is an XRPD pattern of Form L of Compound 1.

FIG. 44 is a DSC curve of Form L of Compound 1.

FIG. 45 is a TGA curve of Form L of Compound 1.

FIG. 46 is a ¹H NMR spectrum of Form L of Compound 1.

FIG. 47 is a moisture sorption curve of Form L of Compound 1.

FIG. 48 is an XRPD pattern of Form M of Compound 1.

Form 49 is a DSC curve of Form M of Compound 1.

Form 50 is a TGA curve of Form M of Compound 1.

Form 51 is a ¹H NMR spectrum of Form M of Compound 1.

FIG. 52 is a XRPD pattern of Form N of Compound 1.

FIG. 53 is a DSC curve of Form N of Compound 1.

FIG. 54 is a TGA curve of Form N of Compound 1.

FIG. 55 is a ¹H NMR spectrum of Form N of Compound 1.

FIG. 56 is an XRPD pattern of Form O of Compound 1.

FIG. 57 is a DSC curve of Form O of Compound 1.

FIG. 58 is a TGA curve of Form O of Compound 1.

FIG. 59 is a XRPD pattern of Form P of Compound 1.

FIG. 60 illustrates the conversion of forms observed from slurry and humidity chamber studies.

### DETAILED DESCRIPTION OF THE INVENTION

The present invention provides novel polymorphs of Compound 1, as well as compositions comprising Compound 1, where at least a portion of Compound 1 is present in the composition in a form selected from the group consisting of crystalline forms (e.g., Form A, Form B, Form C, Form D, Form E, Form F, Form G, Form I, Form J, Form K, Form L, Form M, Form N, Form O and Form P) and an amorphous form (e.g., Amorphous Form).

Also provided are kits and other articles of manufacture with compositions comprising Compound 1 where at least a portion of Compound 1 is present in the composition in a form selected from the group consisting of crystalline forms (e.g., Form A, Form B, Form C, Form D, Form E, Form F, Form G, Form I, Form J, Form K, Form L, Form M, Form N, Form O and Form P) and an amorphous form (e.g., Amorphous Form).

Various methods are also provided including methods of making each of the disclosed forms; methods for manufacturing pharmaceutical compositions comprising Compound 1 where at least a portion of Compound 1 is present in the composition in a form selected from the group consisting of crystalline forms (i.e., Form A, Form B, Form C, Form D, Form E, Form F, Form G, Form I, Form J, Form K, Form L, Form M, Form N, Form O and Form P) and an amorphous form; and methods of using compositions comprising Compound 1 where at least a portion of Compound 1 is present in the composition in a form selected from the group consisting of crystalline forms (e.g., Form A, Form B, Form C, Form D, Form E, Form F, Form G, Form I, Form J, Form K, Form L, Form M, Form N, Form O and Form P) and an amorphous form (e.g., Amorphous Form).

As one will appreciate, depending on how a composition comprising a given compound is produced and then, once produced, how the composition is stored and manipulated, will influence the crystalline content of the composition. Accordingly, it is possible for a composition to comprise no crystalline content or may comprise higher concentrations of crystalline content.

It is further noted that a compound may be present in a given composition in one or more different polymorphic forms, as well as optionally also being present as an amorphous material. This may be the result of (a) physically mixing two or more different polymorphic forms; (b) having two or more different polymorphic forms be generated from crystallization conditions; (c) having all or a portion of a given polymorphic form convert into another polymorphic form; and (d) having all or a portion of a compound in an amorphous state convert into two or more polymorphic forms; as well as for a host of other reasons.

As can be seen, depending on how a composition comprising a compound is prepared, the percentage, by weight, of that compound in a given polymorphic form can vary from 0% to 100%. According to the present invention, compositions are provided where greater than 0.1%, 0.25%, 0.5%, 1%, 5%, 10%, 25%, 50%, 75%, 80%, 85%, 90%, 95%, 97%, or 99% or more of Compound 1 (by weight) is present in the composition in a form selected from the group consisting of Form A, Form B, Form C, Form D, Form E, Form F, Form G, Form I, Form J, Form K, Form L, Form M, Form N, Form O Form P and Amorphous Form.

### Definitions

“Crystalline”, as the term is used herein, refers to a material that contains a specific compound, which may be hydrated and/or solvated, and has sufficient crystalline content to exhibit a discernable diffraction pattern by XRPD or other diffraction techniques. Often, a crystalline material that is obtained by direct crystallization of a compound dissolved in a solution or interconversion of crystals obtained under different crystallization conditions, will have crystals that contain the solvent used in the crystallization, termed a crystalline solvate. Also, the specific solvent system and physical embodiment in which the crystallization is performed, collectively termed crystallization conditions, may result in the crystalline material having physical and chemical properties that are unique to the crystallization conditions, generally due to the orientation of the chemical moieties of the compound with respect to each other within the crystal and/or the predominance of a specific polymorphic form of the compound in the crystalline material.

Depending upon the polymorphic form(s) of the compound that are present in a composition, various amounts of the compound in an amorphous solid state may also be present, either as a side product of the initial crystallization, and/or a product of degradation of the crystals comprising the crystalline material. Thus, crystalline, as the term is used herein, contemplates that the composition may include amorphous content; the presence of the crystalline material among the amorphous material being detectable by, among other methods, the composition having a diffraction pattern with individual, discernable peaks.

The amorphous content of a crystalline material may be increased by grinding or pulverizing the material, which is evidenced by broadening of diffraction and other spectral lines relative to the crystalline material prior to grinding. Sufficient grinding and/or pulverizing may broaden the lines relative to the crystalline material prior to grinding to the extent that the XRPD or other crystal specific spectrum may become undiscernable, making the material substantially amorphous or quasi-amorphous.

Continued grinding would be expected to increase the amorphous content and further broaden the XRPD pattern with the limit of the XRPD pattern being so broadened that it can no longer be discerned above noise. When the XRPD pattern is broadened to the limit of being indiscernible, the material may be considered to no longer be a crystalline material, but instead be wholly amorphous. For material having increased amorphous content and wholly amorphous material, no peaks should be observed that would indicate grinding produces another form.

“Amorphous”, as the term is used herein, refers to a composition comprising a compound that contains too little crystalline content of the compound to yield a diffraction pattern, by XRPD or other diffraction techniques, having individual, discernable peaks. Glassy materials are a type of amorphous material. Glassy materials do not have a true crystal lattice, and technically resemble very viscous non-crystalline liquids. Rather than being true solids, glasses may better be described as quasi-solid amorphous material.

“Broad” or “broadened”, as the term is used herein to describe spectral lines, including XRPD, NMR, IR and Raman spectroscopy lines, is a relative term that relates to the line width of a baseline spectrum. The baseline spectrum is often that of an unmanipulated crystalline form of a specific compound as obtained directly from a given set of physical and chemical conditions, including solvent composition and properties such as temperature and pressure. For example, broadened can be used to describe the spectral lines of a XRPD spectrum of ground or pulverized material comprising a crystalline compound relative to the material prior to grinding. In materials where the constituent molecules, ions or atoms, as solvated or hydrated, are not tumbling rapidly, line broadening is indicative of increased randomness in the orientation of the chemical moieties of the compound, thus indicative of an increased amorphous content. When comparisons are made between crystalline materials obtained via different crystallization conditions, broader spectral lines indicate that the material producing the relatively broader spectral lines has a higher level of amorphous material.

“About” as the term is used herein, refers to an estimate that the actual value falls within 5% of the value cited.

“Forked” as the term is used herein to describe DSC endotherms and exotherms, refers to overlapping endotherms or exotherms having distinguishable peak positions.

### Preparation and Characterization of the Polymorphs

A. Preparation of Compound 1

Various methods may be used to synthesize Compound 1. A representative method for synthesizing Compound 1 is provided in Example 1. It is noted, however, that other synthetic routes may also be used to synthesize Compound 1.

B. Preparation of the Polymorphs of Compound 1

General methods for precipitating and crystallizing a compound may be applied to prepare the various polymorphs described herein. These general methods are known to those skilled in the art of synthetic organic chemistry and pharmaceutical formulation, and are described, for example, by J. March, “Advanced Organic Chemistry: Reactions, Mechanisms and Structure,” 4^{th} Ed. (New York: Wiley-Interscience, 1992).

In general, a given polymorph of a compound may be obtained by direct crystallization of the compound or by crystallization of the compound followed by inter-conversion from another polymorphic form or from an amorphous form. Depending on the method by which a compound is crystallized, the resulting composition may contain different amounts of the compound in crystalline form as opposed to as an amorphous material. Also, the resulting composition may contain differing mixtures of different polymorphic forms of the compound.

Compositions comprising a higher percentage of crystalline content (e.g., forming crystals having fewer lattice defects and proportionately less glassy material) are generally prepared when conditions are used that favor slower crystal formation, including those slowing solvent evaporation and those affecting kinetics. Crystallization conditions may be appropriately adjusted to obtain higher quality crystalline material as necessary. Thus, for example, if poor crystals are formed under an initial set of crystallization conditions, the solvent temperature may be reduced and ambient pressure above the solution may be increased relative to the initial set of crystallization conditions in order to slow crystallization.

Precipitation of a compound from solution, often affected by rapid evaporation of solvent, is known to favor the compound forming an amorphous solid as opposed to crystals. A compound in an amorphous state may be produced by rapidly evaporating solvent from a solvated compound, or by grinding, pulverizing or otherwise physically pressurizing or abrading the compound while in a crystalline state.

Compound 1 as prepared by the method described in Example I may be used as the starting material for preparation of other polymorphic forms. The methods for testing the solubility of Compound 1 are described in Example 3, and the solubilities of Compound 1 in various solvents are summarized in Table 16. Good solubility was observed in dioxane, MeOH, DMF, DMA, NMP, AcOH and EtOH. Poor solubility was observed in acetone, MeCN, MTBe, EtOAc, IPAc, IPA, THF, 2-Me-THF, DCM, MEK, cyclohexane, heptane and water.

Methods by which the various polymorphic forms may be prepared are described in the Examples section. Specific methods by which Form A, Form B, Form C, Form D, Form E, Form F, Form G, Form I, Form J, Form K, Form L, Form M, Form N, Form O and Form P may be prepared are summarized below, including in Tables 17-30, 34, 36a and 36b.

C. Polymorphs of Compound 1

Fifteen crystalline forms and one amorphous solid were identified by conducting a polymorph screen. Described herein are Form A, Form B, Form C, Form D, Form E, Form F, Form G, Form I, Form J, Form K, Form L, Form M, Form N, Form O and Form P and Amorphous Form of Compound 1. As described in greater detail below, Forms B, E, G, and I were found to be solvates of DMA, NMP, DMF, and THF respectively. Forms A, L, M, and N were found to be hydrates where Form A was confirmed to be a monohydrate and Form L was found to be a channel hydrate. The remaining forms were found to be either anhydrates (C, F, J, K, O) or likely anhydrates (D, P). Where possible, the results of each test for each different polymorph are provided.

Various tests were performed in order to physically characterize the polymorphs of Compound 1 including X-ray powder diffraction (XRPD), differential scanning calorimetry (DSC), thermogravimetric analysis (TGA), solution proton nuclear magnetic resonance (¹H-NMR), and moisture sorption and desorption analysis (M S/Des). Detailed experimental conditions for each of the analytical techniques are described in Example 2. The characterization of Forms A, B, C, D, E, F, G, 1, J, K, L, M, N, O, and P and Amorphous Form are described below, as are methods for testing the stabilities of the various forms of Compound 1, and the conditions under which the polymeric forms interconvert are also described below.

1. Form A

Based on the available characterization data, Form A appears to be a monohydrate polymorphic form of Compound 1 that is stable at ambient conditions. Form A was characterized by a variety of techniques, including XRPD, DSC, TGA, ¹H-NMR and moisture sorption analysis. Table 36a summarizes some of these results. Preparation and scale-up studies related to Form A are presented in Examples 6-10. For example, Form A could be obtained successfully from a water re-slurry (e.g., a binary solvent system, such as MeCN/water) for approximately 4-5 hours at ambient temperature.

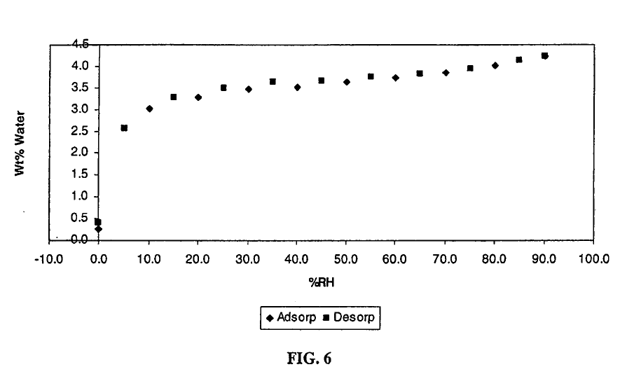

Form A is consistent with a monohydrate based on Karl Fischer (KF) and moisture sorption data (FIG. 6). For example, KF analysis of a sample of Form A showed 3.7% water, consistent with a monohydrate (the theoretical wt % for a monohydrate is 3.2%). KF analysis of another sample of Form A showed 3.1% water before heating and 3.0% water after heating. The moisture sorption curve (FIG. 6) shows the hydrate to be stable from 5 to 90% RH, with a maximum moisture uptake of 4.2 wt % at 90% RH. The experiment did not time out (>4 hours) at any point consistent with the hydrated form being stable during the experiment.

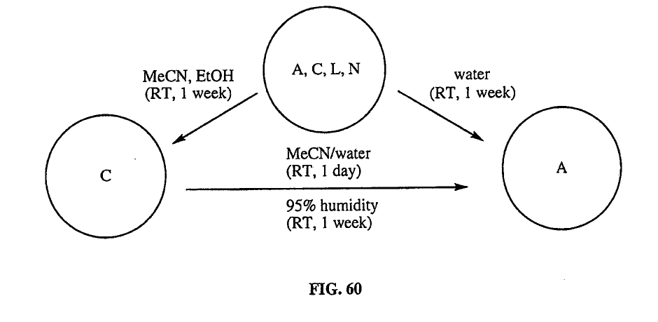

A sample of Form A that was dried for one hour at 80° C. to remove water and XRPD analysis following drying showed a pattern consistent with Form A (Table 38 and Example 12). The equilibration to roughly 1 mole of water under the wide ranges of humidity is consistent with the dehydrated material rapidly reconverting to the hydrated Form A upon exposure to ambient laboratory conditions. Further characterization of Form A by XRPD and KF following heating was performed and further confirmed this assessment. XRPD showed the same pattern before and after heating. Form A was also found to be the isolated form when Forms C, L, or N were slurried in water. See Table 34 and FIG. 60. Solubility measurement results showed similar values for both the DI water and the phosphate buffer slurries after equilibration overnight and 1 week, indicating 3 to 4 mg/mL as shown in Table 37. Form A also did not show a change in form upon exposure to 0 and 95% RH for one week, further consistent with a stable monohydrate form as shown in Table 38.

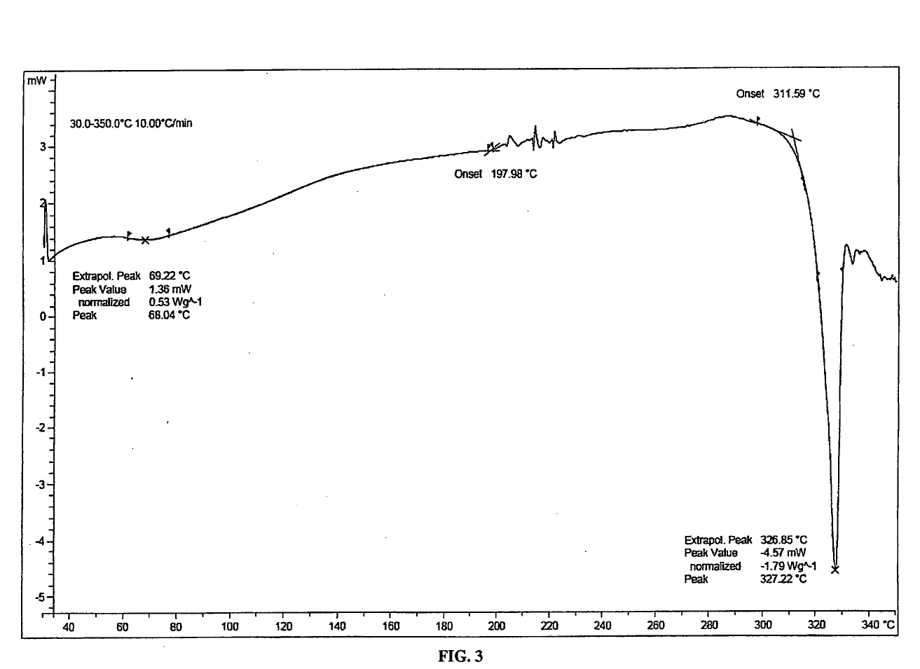

FIG. 2 shows a characteristic XRPD spectrum (CuKα, λ=1.5418 Å) of Form A. The XRPD pattern confirms that Form A is crystalline. Major X-Ray diffraction lines expressed in °2θ and their relative intensities are summarized in Table 1.

TABLE 1
Characteristic XRPD Peaks (CuKα) of Form A

| Peak No. | 2θ (°) | d-spacing | Intensity | I/Iₒ |
|---|---|---|---|---|
| 1 | 4.6000 | 19.19426 | 144 | 2301 |
| 2 | 5.2024 | 16.97298 | 2211 | 15919 |
| 3 | 8.3967 | 10.52186 | 128 | 1187 |
| 4 | 10.2853 | 8.59367 | 938 | 9850 |
| 5 | 14.9200 | 5.93296 | 97 | 939 |
| 6 | 15.2800 | 5.79398 | 192 | 737 |
| 7 | 15.5306 | 5.70105 | 728 | 4707 |
| 8 | 16.2800 | 5.44027 | 242 | 2573 |
| 9 | 16.8000 | 5.27303 | 493 | 2925 |
| 10 | 17.0400 | 5.19930 | 865 | 6197 |
| 11 | 18.0457 | 4.91174 | 84 | 698 |
| 12 | 19.0254 | 4.66096 | 224 | 1633 |
| 13 | 19.8973 | 4.45864 | 484 | 5068 |
| 14 | 20.4800 | 4.33308 | 925 | 6275 |
| 15 | 20.7600 | 4.27527 | 673 | 4921 |
| 16 | 21.2000 | 4.18752 | 100 | 840 |
| 17 | 21.8213 | 4.06968 | 110 | 707 |
| 18 | 22.4272 | 3.96108 | 432 | 3171 |
| 19 | 23.2097 | 3.82927 | 148 | 1069 |
| 20 | 23.7324 | 3.74611 | 274 | 2115 |
| 21 | 24.3450 | 3.65321 | 232 | 1396 |
| 22 | 24.9029 | 3.57262 | 148 | 1107 |
| 23 | 25.2469 | 3.52471 | 167 | 920 |
| 24 | 25.9630 | 3.42910 | 418 | 3520 |
| 25 | 26.8560 | 3.31707 | 383 | 3177 |
| 26 | 27.2400 | 3.27117 | 120 | 972 |
| 27 | 28.2240 | 3.15932 | 76 | 740 |
| 28 | 30.6000 | 2.91921 | 67 | 411 |
| 29 | 30.8400 | 2.89703 | 68 | 963 |
| 30 | 32.1070 | 2.78554 | 99 | 588 |
| 31 | 32.7664 | 2.73098 | 86 | 1644 |
| 32 | 36.0157 | 2.49169 | 92 | 550 |
| 33 | 36.8551 | 2.43685 | 68 | 1234 |
| 34 | 39.2195 | 2.29521 | 203 | 1876 |

This unique set of XRPD peak positions or a subset thereof can be used to identify Form A. One such subset comprises peaks at about 5.2, 10.3 and 20.5°2θ. Another subset comprises peaks comprises peaks at about 15.5, 17.0 and 19.9°2θ.

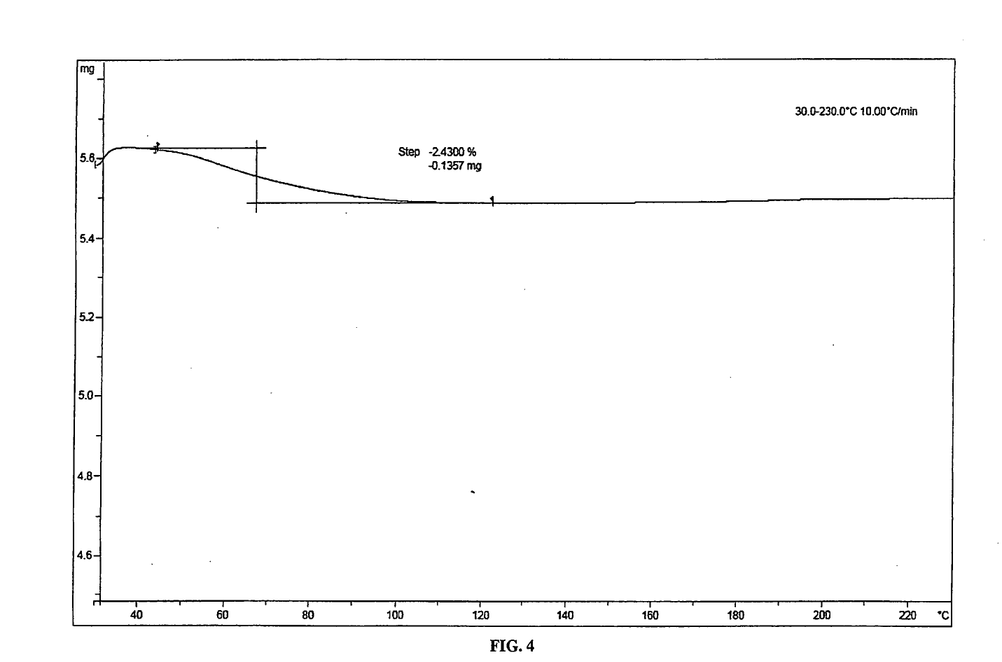

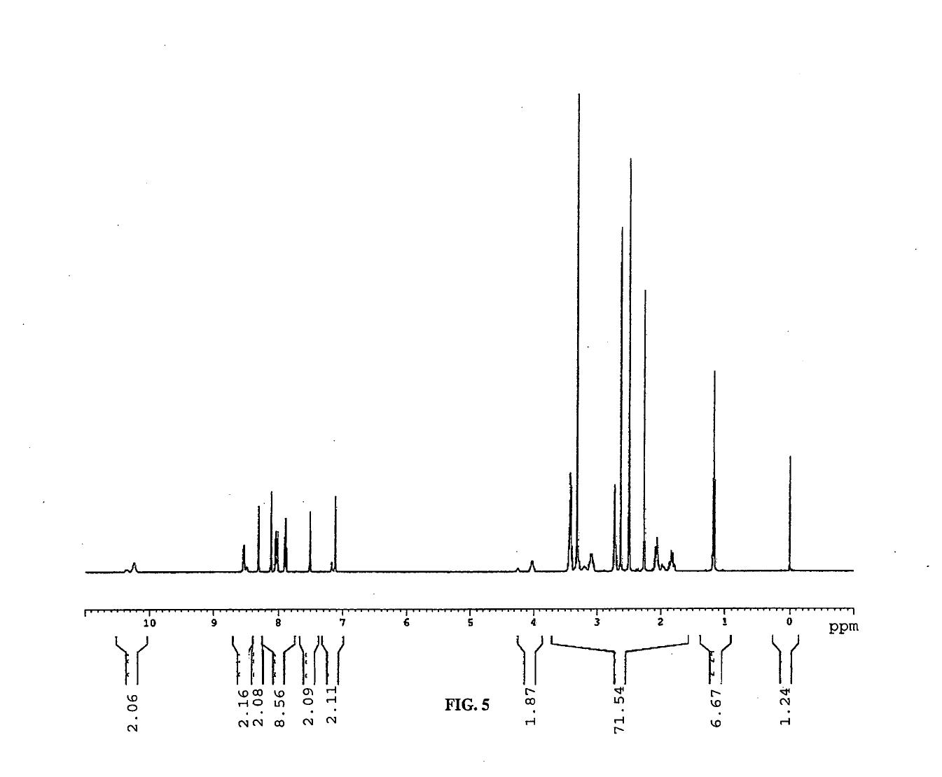

FIG. 3 shows a characteristic DSC thermogram of Form A. An endotherm was observed at approximately 327° C. (peak maximum). FIG. 4 is a TGA thermogram of Form A, showing a weight loss of approximately 2.4% at a temperature below 100° C. The theoretical weight loss for a monohydrate is 3.2%.

Form A was further characterized by solution ¹H NMR. The spectrum is reported in FIG. 5. Chemical assignments were not performed; however, the spectra are consistent with the known chemical structure of Compound 1.

Further details related to the preparation and characterization of Form A are presented below in the Examples section.

2. Form B

Based on the available characterization data, Form B appears to be a DMF solvate polymorphic form of Compound 1. Form B was characterized by a variety of techniques, including XRPD, DSC, TGA, and ¹H-NMR. Table 36b summarizes some of these results.

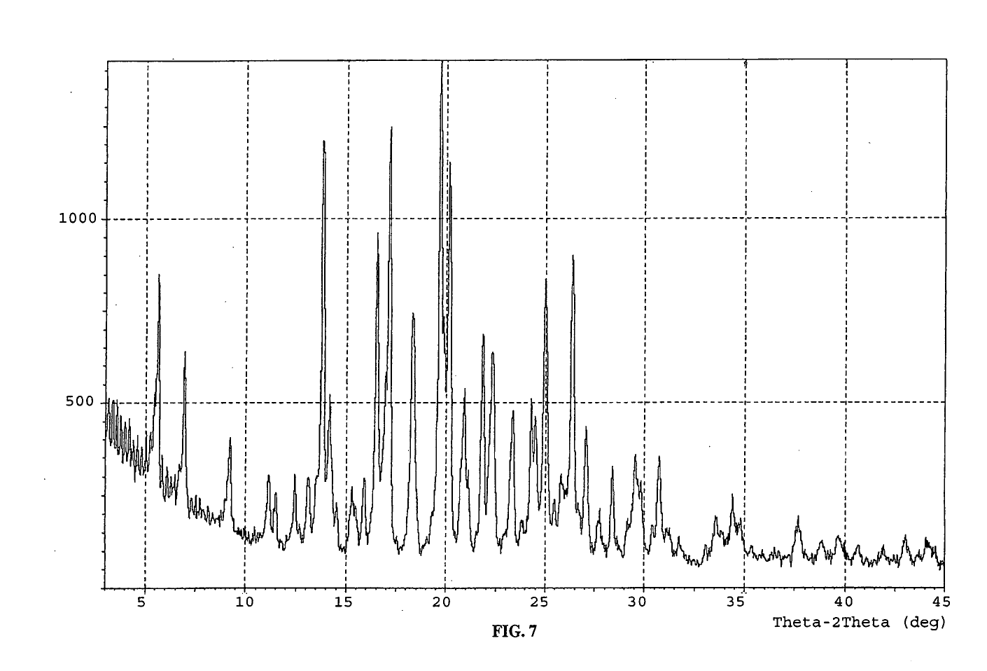

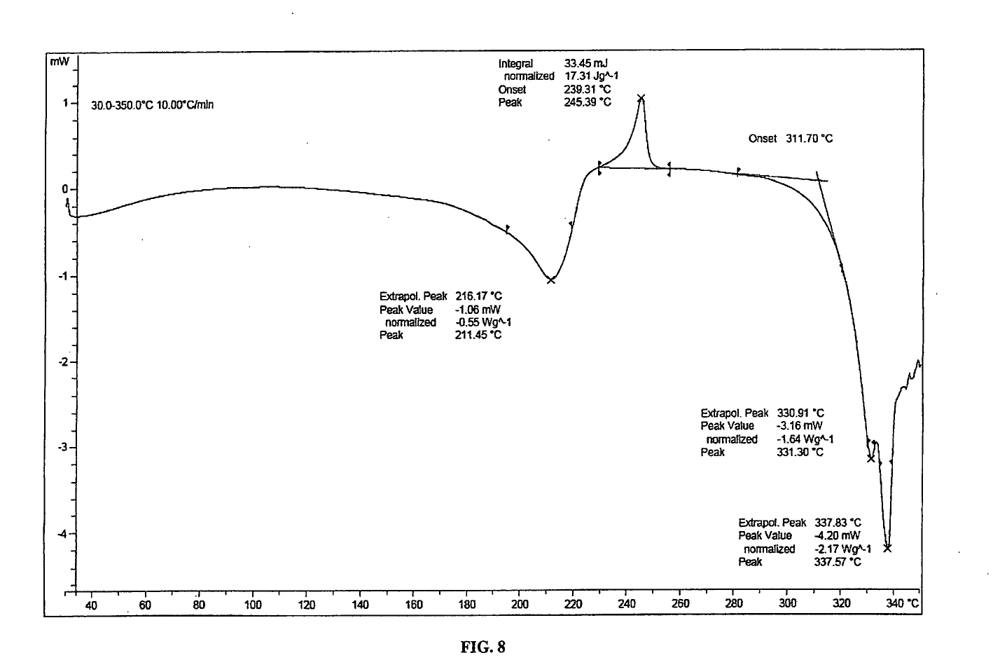

FIG. 7 shows a characteristic XRPD spectrum (CuKα, λ=1.5418 Å) of Form B. The XRPD pattern confirms that Form B is crystalline. Major X-Ray diffraction lines expressed in °2θ and their relative intensities are summarized in Table 2.

TABLE 2
Characteristic XRPD Peaks (CuKα) of Form B

| Peak No. | 2θ (°) | d-spacing | Intensity | I/Iₒ |
|---|---|---|---|---|
| 1 | 3.1200 | 28.29512 | 36 | 52 |
| 2 | 4.2012 | 21.01534 | 46 | 346 |
| 3 | 5.2400 | 16.85127 | 74 | 454 |
| 4 | 5.5845 | 15.81249 | 363 | 2252 |
| 5 | 6.9185 | 12.76628 | 280 | 1756 |
| 6 | 8.9600 | 9.86159 | 50 | 280 |
| 7 | 9.1743 | 9.63172 | 178 | 977 |
| 8 | 11.1284 | 7.94443 | 140 | 955 |
| 9 | 11.4724 | 7.70698 | 111 | 599 |
| 10 | 12.4362 | 7.11177 | 134 | 909 |
| 11 | 13.1090 | 6.74824 | 155 | 865 |
| 12 | 13.4800 | 6.56334 | 129 | 844 |
| 13 | 13.7939 | 6.41468 | 860 | 4077 |
| 14 | 14.1435 | 6.25689 | 286 | 2040 |
| 15 | 14.5200 | 6.09549 | 88 | 400 |
| 16 | 15.2800 | 5.79398 | 106 | 692 |
| 17 | 15.4800 | 5.71957 | 64 | 316 |
| 18 | 15.8606 | 5.58316 | 136 | 684 |
| 19 | 16.2000 | 5.46695 | 50 | 170 |
| 20 | 16.4932 | 5.37042 | 629 | 4273 |
| 21 | 16.8400 | 5.26059 | 240 | 0 |
| 22 | 17.1041 | 5.17996 | 863 | 5415 |
| 23 | 18.0000 | 4.92411 | 39 | 160 |
| 24 | 18.2901 | 4.84665 | 539 | 3482 |
| 25 | 19.2400 | 4.60946 | 57 | 282 |
| 26 | 19.6698 | 4.50970 | 1013 | 6807 |
| 27 | 20.1135 | 4.41120 | 818 | 4984 |
| 28 | 20.8577 | 4.25546 | 303 | 1985 |
| 29 | 21.1200 | 4.20320 | 110 | 456 |
| 30 | 21.8030 | 4.07305 | 438 | 2257 |
| 31 | 22.2845 | 3.98612 | 420 | 3169 |
| 32 | 23.2986 | 3.81486 | 276 | 1641 |
| 33 | 23.7908 | 3.73704 | 32 | 104 |
| 34 | 24.2800 | 3.66284 | 260 | 1229 |
| 35 | 24.4800 | 3.63337 | 238 | 1274 |
| 36 | 24.9602 | 3.56454 | 558 | 3435 |
| 37 | 25.4395 | 3.49846 | 68 | 339 |
| 38 | 25.8000 | 3.45039 | 120 | 892 |
| 39 | 26.3343 | 3.38158 | 631 | 3405 |
| 40 | 26.6800 | 3.33855 | 77 | 641 |
| 41 | 27.0381 | 3.29514 | 258 | 1441 |
| 42 | 27.6966 | 3.21827 | 83 | 418 |
| 43 | 28.3728 | 3.14309 | 174 | 788 |
| 44 | 29.1200 | 3.06412 | 60 | 240 |
| 45 | 29.5200 | 3.02350 | 199 | 1568 |
| 46 | 29.8400 | 2.99180 | 113 | 672 |
| 47 | 30.3757 | 2.94025 | 61 | 277 |

This unique set of XRPD peak positions or a subset thereof can be used to identify Form B. One such subset comprises peaks at about 13.8, 17.1 and 19.7°2θ. Another subset comprises peaks at about 16.5, 20.1 and 25.0°2θ.

FIG. 8 shows a characteristic DSC thermogram of Form B, showing multiple events, with an endotherm observed near the temperature range observed for bound weight loss by TGA and followed by an exothermic event consistent with re-crystallization to an anhydrous form. The first endotherm is centered at about 211° C.; the second endotherm is forked having peaks at about 331° C. and at about 338° C. The exotherm is centered at about 245° C.

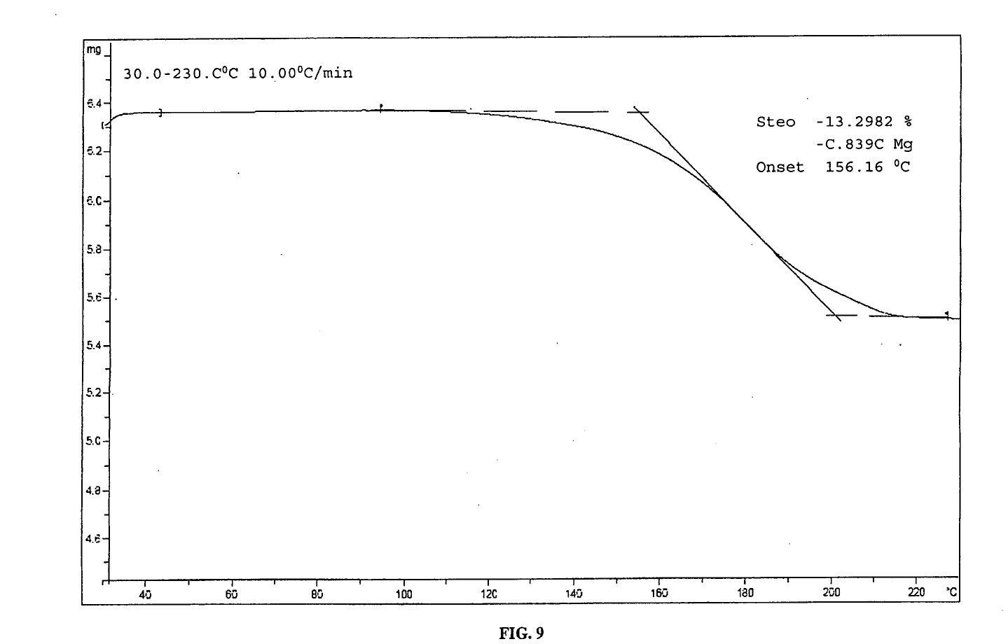

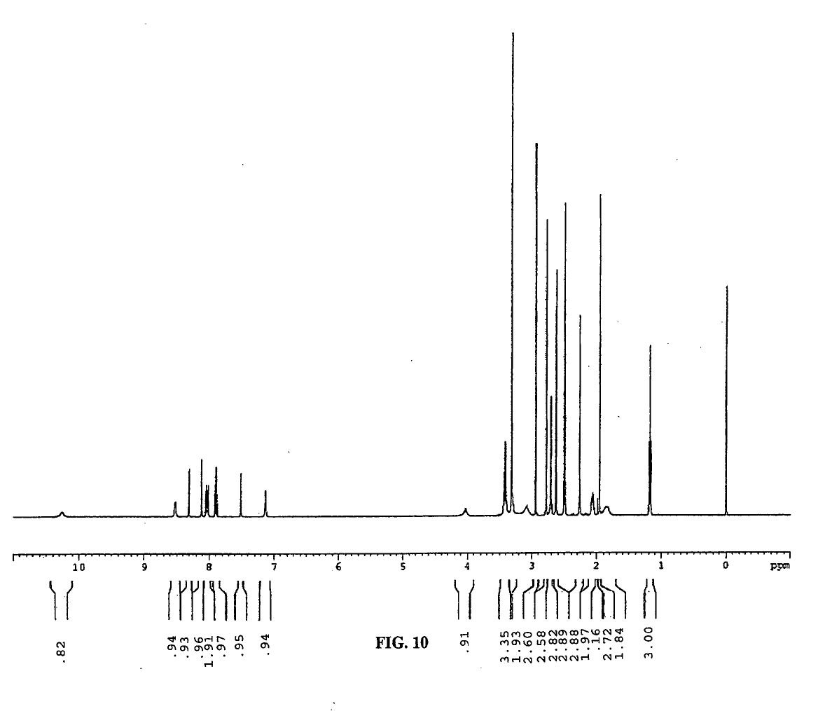

FIG. 9 is a TGA thermogram of Form B.

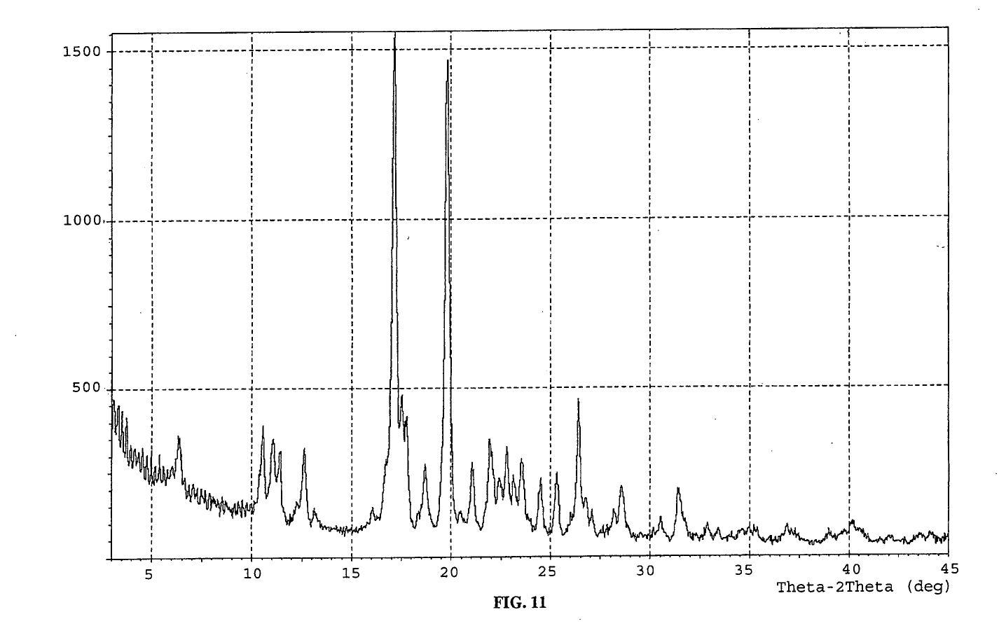

Form B was further characterized by solution ¹H NMR. The spectrum is reported in FIG. 10. The spectrum is consistent with one molar equivalent of solvent present, as well as the known chemical structure of Compound 1.

Further details related to the preparation and characterization of Form B are presented below in the Examples section.

3. Form C

Based on the available characterization data, Form C appears to be an anhydrous polymeric form of Compound 1 that is stable under ambient non-aqueous conditions. Form C can be prepared by slurrying Form A in anhydrous MeCN and MeOH.

Under humid conditions, Form C can be converted to Form A. For example, Form C can be converted to Form A after equilibrating at 95% RH (% relative humidity) for one week at ambient temperature (FIG. 60).

Form C is consistent with an anhydrate based on KF and moisture sorption data showing 1.4% water where 3.2% is theoretical for a monohydrate. The moisture sorption curve showed Form C to be slightly hygroscopic, with a maximum water uptake of 1.9% at 90% RH. The experiment did not time out (>4 hours) at any point and hysteresis was not observed upon desorption. After drying the material at 80° C. for one hour to remove water the sample was analyzed by XRPD and showed a pattern consistent to the starting form. Form C was found to be a stable anhydrate form in non-aqueous environments based on the results of slurry studies presented in Table 34. Form C converted to the monohydrate Form A in water slurries as well as in acetonitrile/water slurries at different ratios (Tables 34 and 35). The humidity chamber study showed that Form C converted to Form A at 95% RH after one week (Table 38).

Form C was characterized by several techniques including XRPD, DSC, TGA, ¹H-NMR and moisture sorption analysis. Table 36a summarizes some of these results. FIG. 11 shows a characteristic XRPD spectrum (CuKα, λ=1.5418 Å) of Form C. The XRPD pattern confirms that Form C is crystalline. Major X-Ray diffraction lines expressed in °2θ and their relative intensities are summarized in Table 3.

TABLE 3
Characteristic XRPD Peaks (CuKα) of Form C

| Peak No. | 2θ (°) | d-spacing | Intensity | I/Iₒ |
|---|---|---|---|---|
| 1 | 3.3217 | 26.57743 | 50 | 259 |
| 2 | 3.5296 | 25.01242 | 36 | 152 |
| 3 | 4.3562 | 20.26793 | 35 | 216 |
| 4 | 4.5614 | 19.35660 | 41 | 189 |
| 5 | 5.3966 | 16.36262 | 36 | 204 |
| 6 | 6.0000 | 14.71837 | 41 | 364 |
| 7 | 6.3449 | 13.91905 | 117 | 890 |
| 8 | 10.5188 | 8.40343 | 183 | 1392 |
| 9 | 11.0558 | 7.99643 | 182 | 1275 |
| 10 | 11.4000 | 7.75576 | 144 | 831 |
| 11 | 12.6166 | 7.01048 | 162 | 1414 |
| 12 | 16.0235 | 5.52677 | 42 | 397 |
| 13 | 16.6400 | 5.32337 | 124 | 1064 |
| 14 | 17.1193 | 5.17539 | 1147 | 9431 |
| 15 | 17.5200 | 5.05792 | 294 | 0 |
| 16 | 17.7200 | 5.00128 | 258 | 2227 |
| 17 | 18.6642 | 4.75034 | 138 | 1167 |
| 18 | 19.4400 | 4.56248 | 88 | 492 |
| 19 | 19.7870 | 4.48325 | 1108 | 7932 |
| 20 | 20.4216 | 4.34534 | 36 | 451 |
| 21 | 21.0406 | 4.21888 | 148 | 911 |
| 22 | 21.9798 | 4.04069 | 199 | 2076 |
| 23 | 22.3600 | 3.97283 | 114 | 0 |
| 24 | 22.7737 | 3.90159 | 175 | 1653 |
| 25 | 23.1517 | 3.83874 | 113 | 761 |
| 26 | 23.5362 | 3.77689 | 158 | 1131 |
| 27 | 24.4723 | 3.63449 | 116 | 692 |
| 28 | 25.2931 | 3.51838 | 140 | 822 |
| 29 | 25.9600 | 3.42949 | 42 | 261 |
| 30 | 26.3864 | 3.37503 | 309 | 1819 |
| 31 | 26.7600 | 3.32875 | 92 | 637 |
| 32 | 27.0579 | 3.29277 | 58 | 308 |
| 33 | 28.1746 | 3.16475 | 60 | 500 |
| 34 | 28.5565 | 3.12329 | 119 | 977 |
| 35 | 30.4944 | 2.92907 | 50 | 421 |
| 36 | 31.4279 | 2.84417 | 127 | 997 |
| 37 | 31.7600 | 2.81518 | 39 | 247 |
| 38 | 36.8369 | 2.43801 | 40 | 436 |
| 39 | 40.1507 | 2.24410 | 47 | 863 |

This unique set of XRPD peak positions or a subset thereof can be used to identify Form C. One such subset comprises peaks at about 17.1, 19.8 and 26.4°2θ. Another subset comprises peaks at about 17.7 and 22.0°2θ.

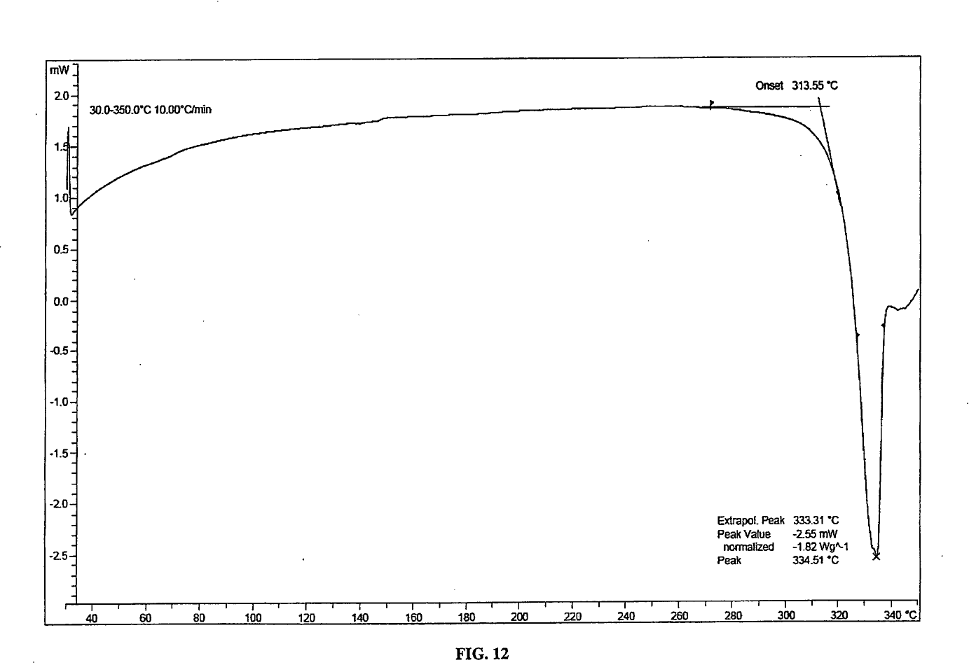

FIG. 12 shows a characteristic DSC thermogram of Form C. An endotherm which onsets at about 314° C. and centered from about 332° C. to about 336° C. and the peak maximum was observed at approximately 335° C.

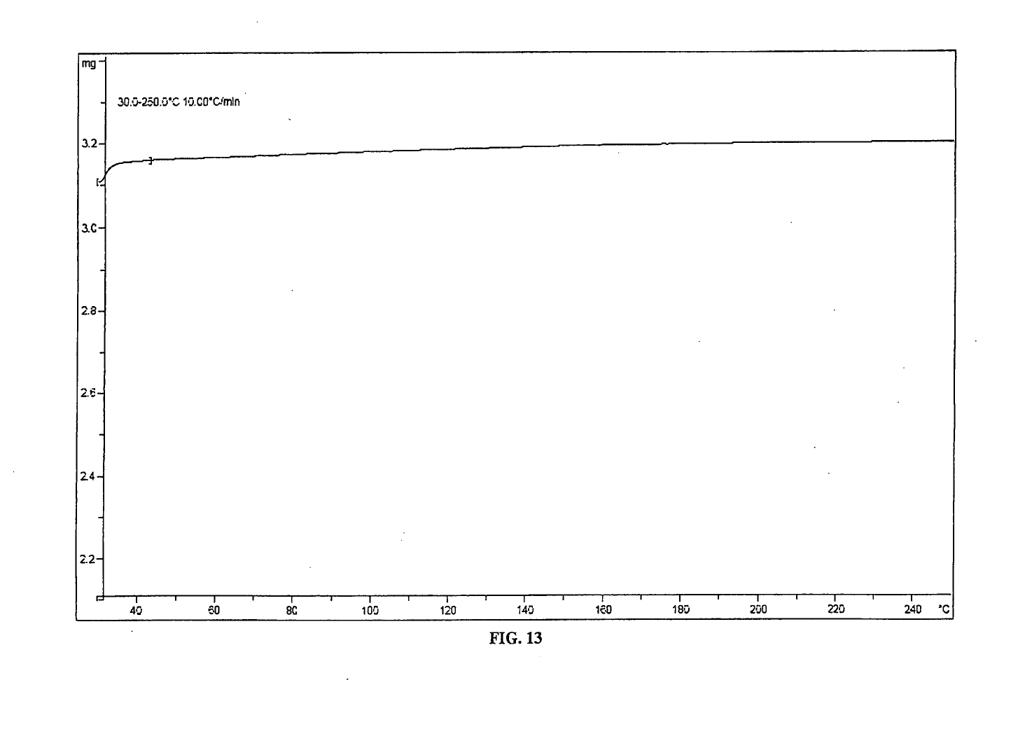

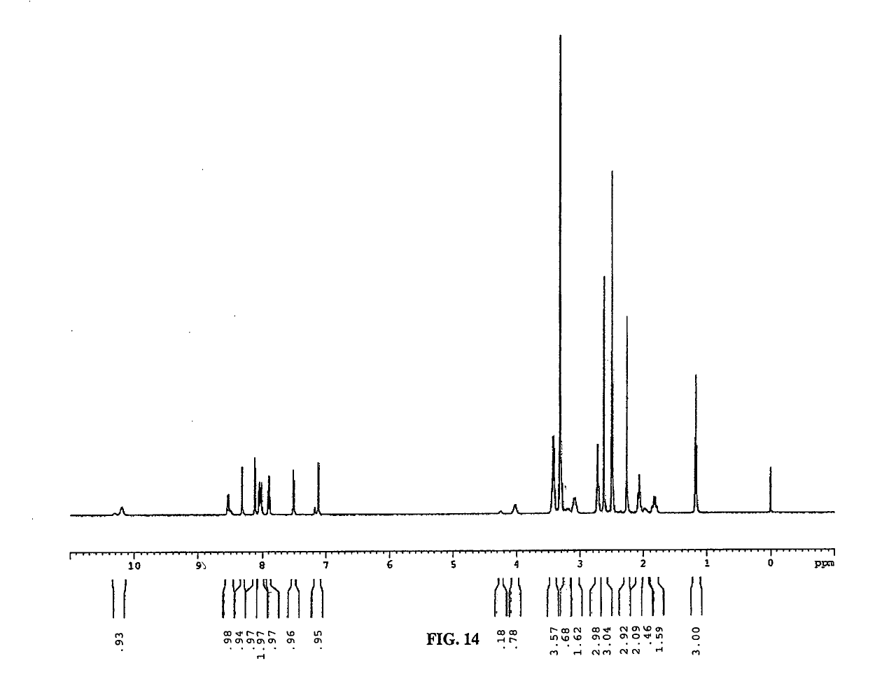

FIG. 13 is a TGA thermogram of Form C. TGA analysis showed no weight loss or only small weight losses likely due to residual solvents.

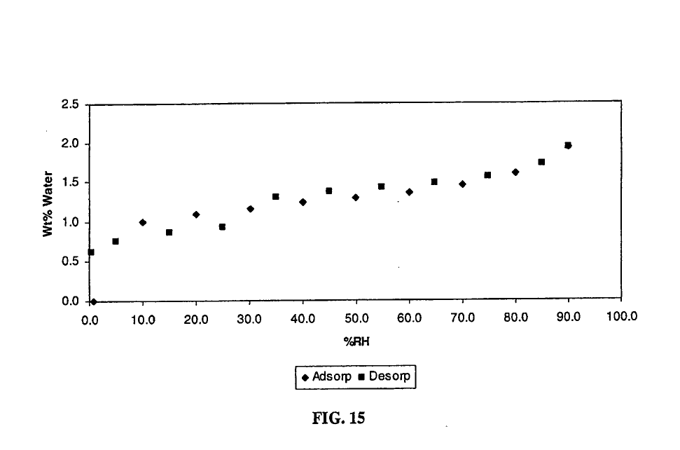

Form C was further characterized by solution ¹H NMR. The spectrum is reported in FIG. 14. Chemical assignments were not performed; however, the spectra are consistent with the known chemical structure of Compound 1. A moisture sorption/desorption analysis is shown in FIG. 15.

Further details related to the preparation and characterization of Form C are presented below in the Examples section.

4. Form D

Based on the available characterization data, Form D appears to be an anhydrous polymorphic form of Compound 1. Form D was characterized by techniques including XRPD, DSC, TGA, and ¹H-NMR. Table 36a summarizes some of these results.

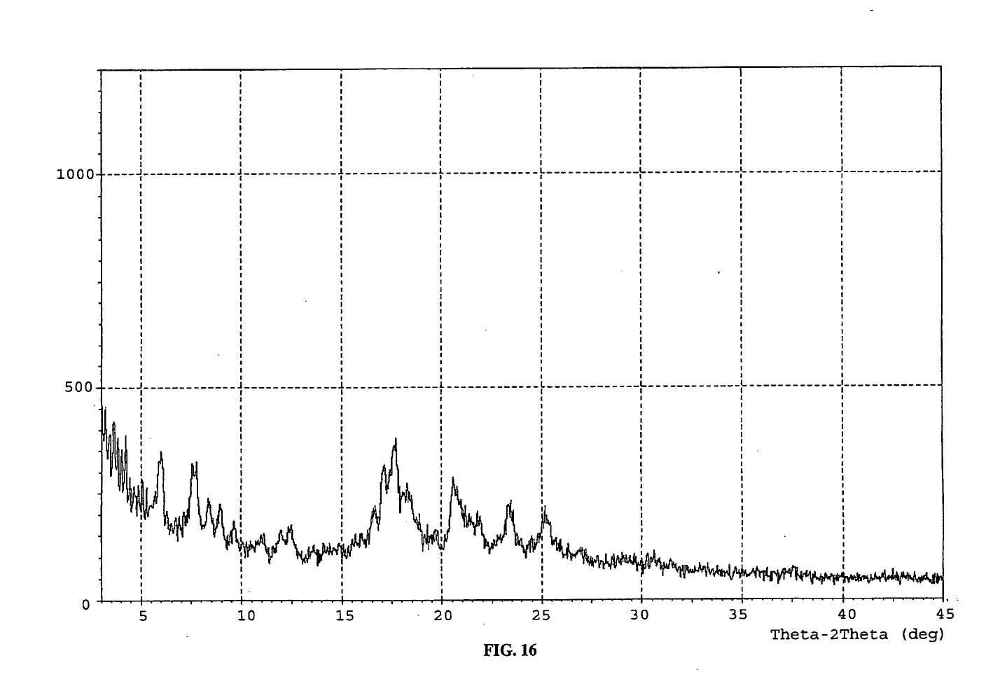

FIG. 16 shows a characteristic XRPD spectrum (CuKα, λ=1.5418 Å) of Form D. The XRPD pattern confirms that Form D is crystalline. Major X-Ray diffraction lines expressed in °2θ and their relative intensities are summarized in Table 4.

TABLE 4
Characteristic XRPD Peaks (CuKα) of Form D

| Peak No. | 2θ (°) | d-spacing | Intensity | I/Iₒ |
|---|---|---|---|---|
| 1 | 5.8720 | 15.03892 | 49 | 1203 |
| 2 | 7.7510 | 11.39688 | 67 | 1686 |
| 3 | 8.8400 | 9.99519 | 27 | 729 |
| 4 | 10.7800 | 8.20039 | 7 | 70 |
| 5 | 12.2700 | 7.20772 | 30 | 753 |
| 6 | 14.4000 | 6.14602 | 7 | 102 |
| 7 | 17.5948 | 5.03658 | 143 | 6341 |
| 8 | 20.8766 | 4.25165 | 78 | 2675 |
| 9 | 23.3666 | 3.80392 | 43 | 994 |
| 10 | 25.1866 | 3.53301 | 43 | 1180 |
| 11 | 26.8200 | 3.32144 | 8 | 145 |
| 12 | 29.2000 | 3.05590 | 8 | 120 |
| 13 | 30.8200 | 2.89887 | 7 | 177 |
| 14 | 37.5200 | 2.39518 | 8 | 177 |

This unique set of XRPD peak positions or a subset thereof can be used to identify Form D. One such subset comprises peaks at about 7.8, 17.6, and 20.9°2θ. Another subset comprises peaks at about 5.9 and 25.2°2θ.

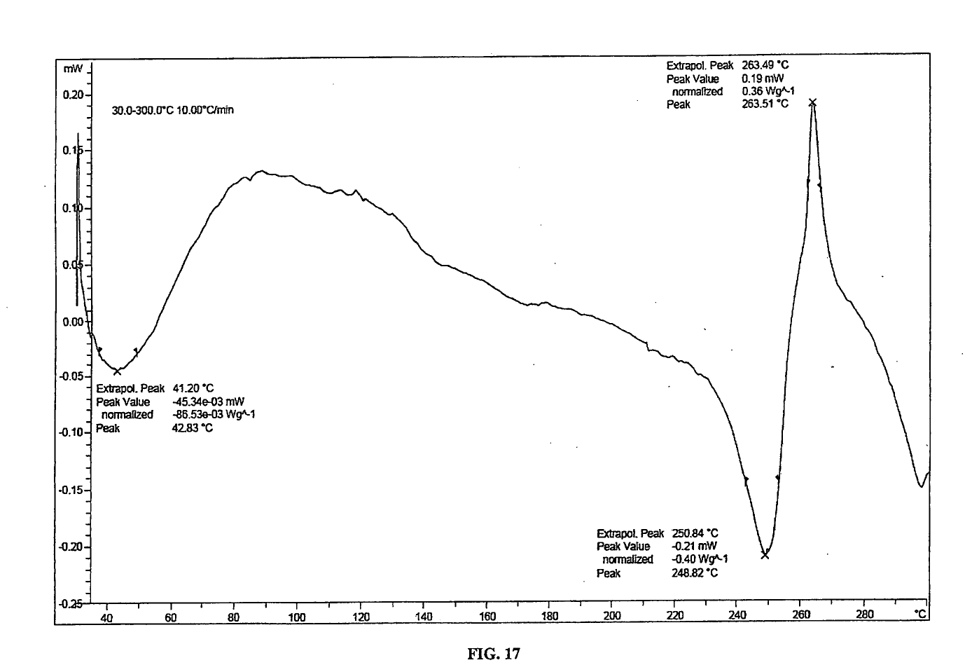

FIG. 17 shows a characteristic DSC thermogram of Form D. An endothermic event centered from about 245° C. to about 255° C. with peak maximum at about 249° C. was observed. An exothermic event which was centered at about 264° C. was also observed.

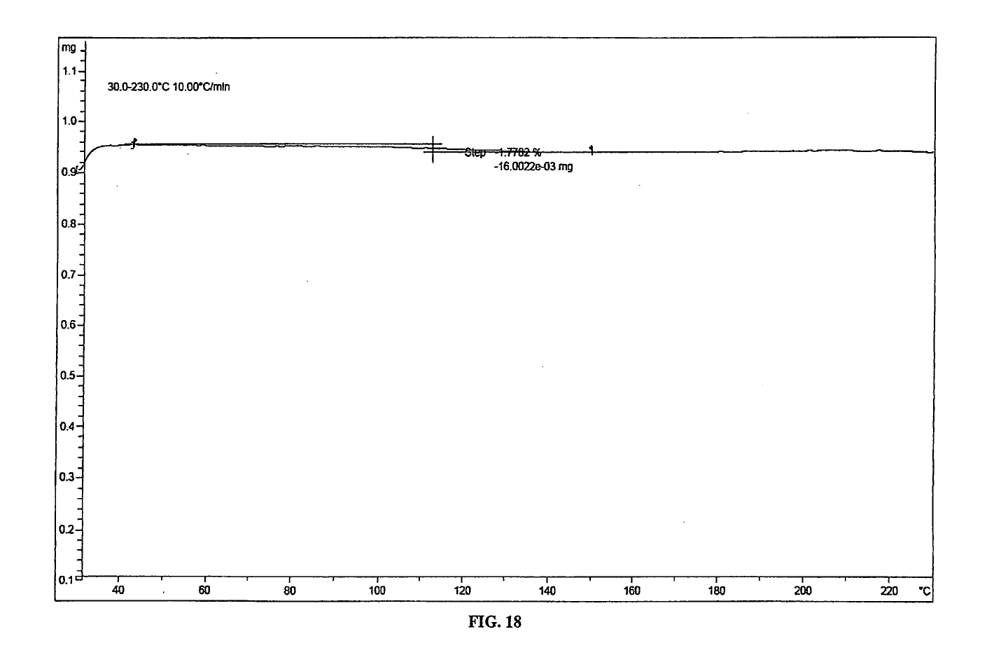

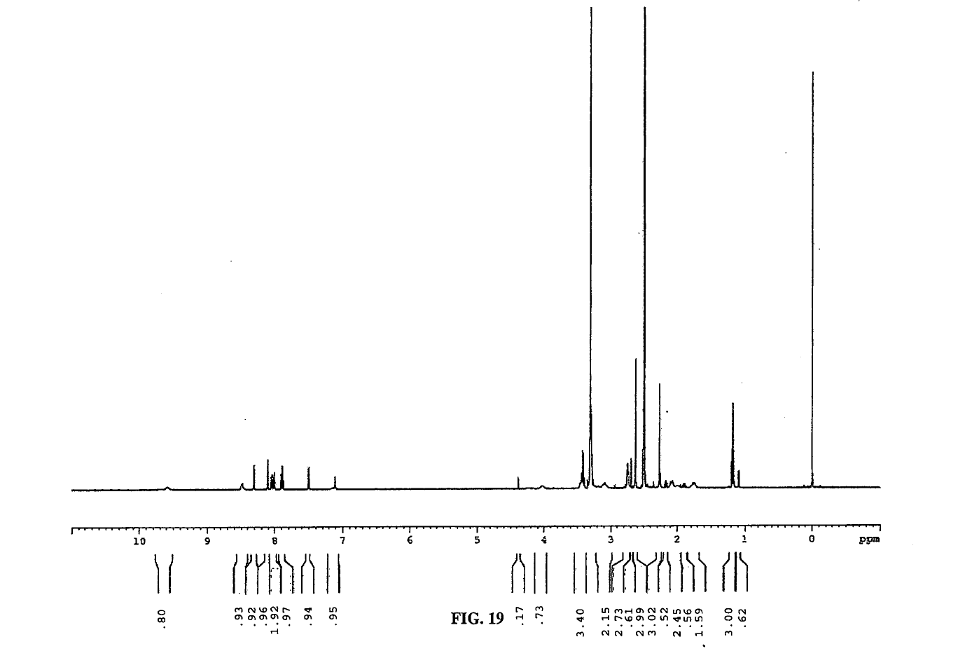

FIG. 18 is a TGA thermogram of Form D. TGA analysis showed no weight loss or only small weight losses likely due to residual solvents.

Form D was further characterized by solution ¹H NMR. The spectrum is reported in FIG. 19. Chemical assignments were not performed; however, the spectra are consistent with the known chemical structure of Compound 1.

Further details related to the preparation and characterization of Form D are presented below in the Examples section.

5. Form E

Based on the available characterization data, Form E appears to be a NMP solvate polymorphic form of Compound 1. Form E was characterized by techniques including XRPD, DSC, TGA, and ¹H-NMR. Table 36b summarizes some of these results.

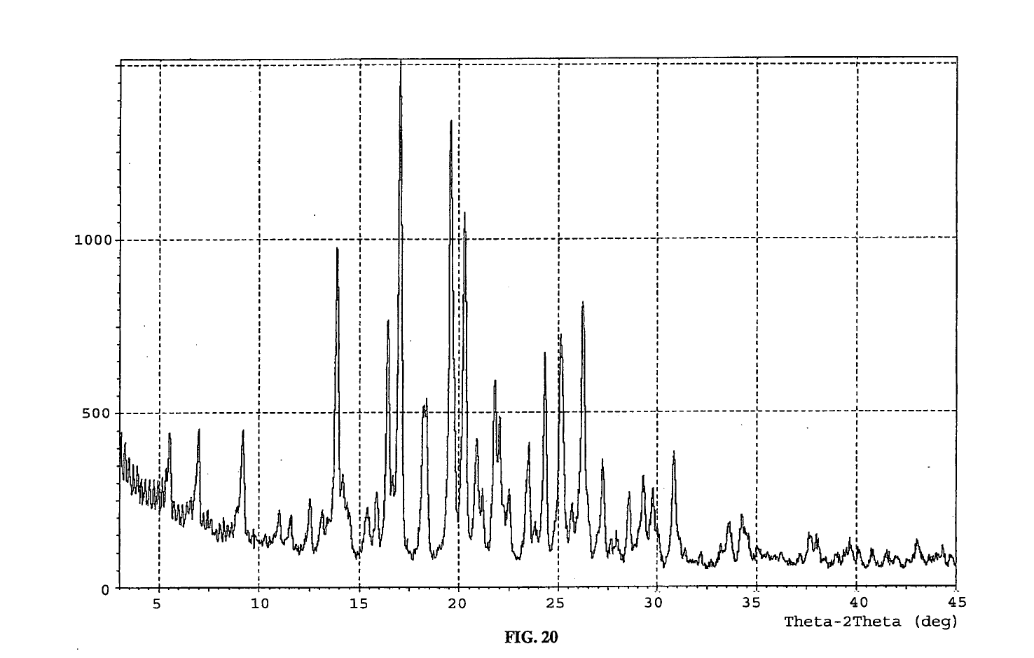

FIG. 20 shows a characteristic XRPD spectrum (CuKα, λ=1.5418 Å) of Form E. The XRPD pattern confirms that Form E is crystalline. Major X-Ray diffraction lines expressed in °2θ and their relative intensities are summarized in Table 5.

TABLE 5
Characteristic XRPD Peaks (CuKα) of Form E

| Peak No. | 2θ (°) | d-spacing | Intensity | I/Iₒ |
|---|---|---|---|---|
| 1 | 3.2796 | 26.91851 | 43 | 161 |
| 2 | 3.4796 | 25.37172 | 34 | 179 |
| 3 | 4.9246 | 17.92979 | 44 | 275 |
| 4 | 5.4866 | 16.09441 | 168 | 1284 |
| 5 | 6.9449 | 12.71781 | 203 | 1689 |
| 6 | 8.8400 | 9.99519 | 56 | 343 |
| 7 | 9.1607 | 9.64598 | 228 | 1460 |
| 8 | 10.9822 | 8.04986 | 70 | 468 |
| 9 | 11.5489 | 7.65610 | 66 | 425 |
| 10 | 12.5213 | 7.06363 | 110 | 629 |
| 11 | 13.1242 | 6.74046 | 84 | 491 |
| 12 | 13.4000 | 6.60234 | 65 | 453 |
| 13 | 13.8611 | 6.38373 | 648 | 4350 |
| 14 | 14.1600 | 6.24964 | 172 | 0 |
| 15 | 14.5200 | 6.09549 | 78 | 1225 |
| 16 | 15.3611 | 5.76357 | 96 | 732 |
| 17 | 15.8393 | 5.59062 | 130 | 755 |
| 18 | 16.4050 | 5.39909 | 520 | 3202 |
| 19 | 17.0185 | 5.20582 | 1073 | 6834 |
| 20 | 17.9600 | 4.93498 | 50 | 188 |
| 21 | 18.2600 | 4.85458 | 350 | 3028 |
| 22 | 19.5778 | 4.53068 | 976 | 5631 |
| 23 | 19.8000 | 4.48034 | 236 | 1186 |
| 24 | 20.2445 | 4.38295 | 755 | 4547 |
| 25 | 20.8611 | 4.25478 | 253 | 1612 |
| 26 | 21.1600 | 4.19535 | 119 | 669 |
| 27 | 21.7900 | 4.07545 | 401 | 2126 |
| 28 | 22.0000 | 4.03702 | 295 | 1689 |
| 29 | 22.4800 | 3.95190 | 142 | 1162 |
| 30 | 23.4391 | 3.79231 | 236 | 1552 |
| 31 | 23.8134 | 3.73355 | 66 | 518 |
| 32 | 24.3019 | 3.65959 | 422 | 2213 |
| 33 | 24.7600 | 3.59291 | 52 | 240 |
| 34 | 25.1167 | 3.54269 | 509 | 3622 |
| 35 | 25.6577 | 3.46920 | 108 | 622 |
| 36 | 26.2135 | 3.39689 | 597 | 3619 |
| 37 | 26.5200 | 3.35833 | 84 | 630 |
| 38 | 26.9200 | 3.30933 | 41 | 186 |
| 39 | 27.1968 | 3.27627 | 211 | 1123 |
| 40 | 27.6243 | 3.22653 | 41 | 156 |
| 41 | 27.9202 | 3.19300 | 48 | 256 |
| 42 | 28.5154 | 3.12770 | 160 | 893 |
| 43 | 29.2544 | 3.05035 | 187 | 1398 |
| 44 | 29.7186 | 3.00375 | 147 | 960 |
| 45 | 30.0400 | 2.97234 | 54 | 308 |
| 46 | 30.8020 | 2.90052 | 263 | 1475 |
| 47 | 31.1200 | 2.87160 | 50 | 348 |

This unique set of XRPD peak positions or a subset thereof can be used to identify Form E. One such subset comprises peaks at about 17.0, 19.6 and 20.2°2θ. Another subset comprises peaks at about 13.9, 25.1 and 26.2°2θ.

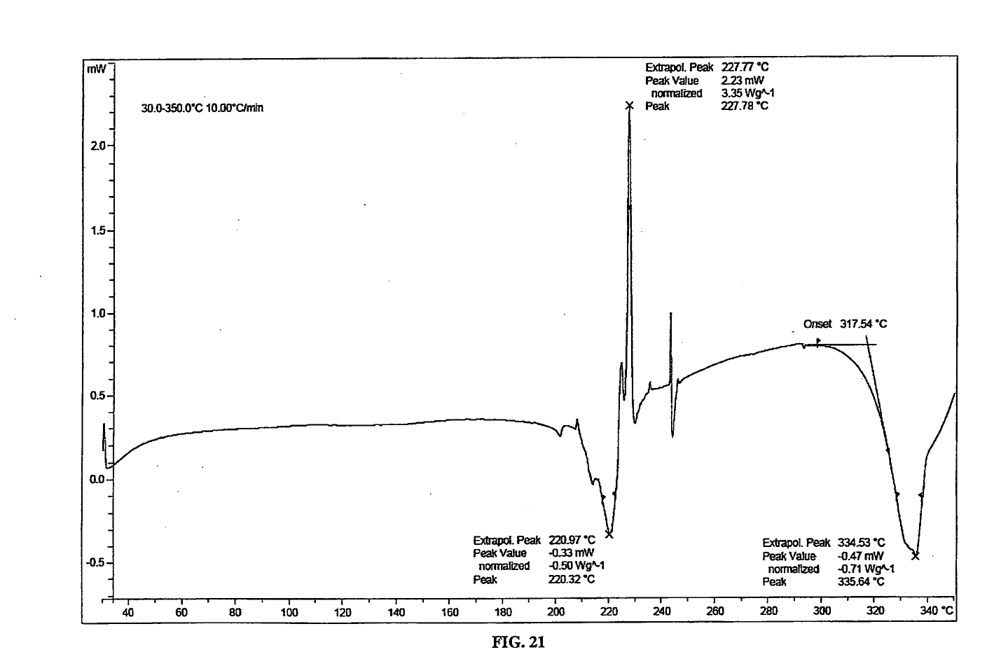

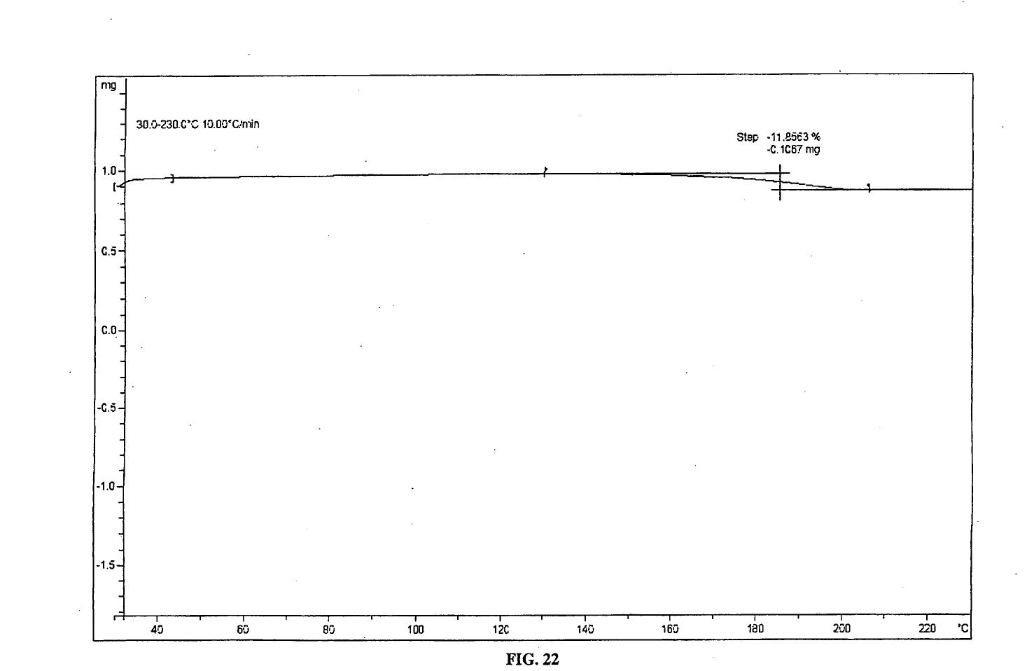

FIG. 21 shows a characteristic DSC thermogram of Form E, showing multiple events, with an endotherm observed near the temperature range observed for bound weight loss by TGA and followed by an exothermic event consistent with re-crystallization to an anhydrous form. The first endothermic event was centered at approximately 220° C. (peak maximum). The second endothermic event onset at about 318° C. and was centered at 336°. The exothermic event was centered at about 228° C. (peak maximum).

FIG. 22 is a TGA thermogram of Form E.

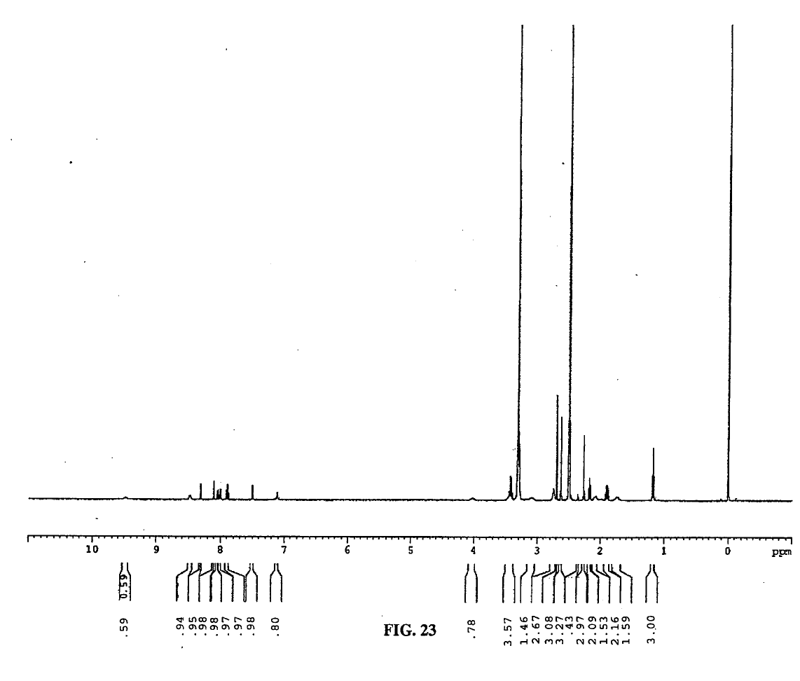

Form E was further characterized by solution ¹H NMR. The spectrum is reported in FIG. 23. The spectrum is consistent with one molar equivalent of solvent present, as well as the known chemical structure of Compound 1.

Further details related to the preparation and characterization of Form E are presented below in the Examples section.

6. Form F

Based on the available characterization data, Form F appears to be a desolvate polymorphic form of Compound 1. Form F can be observed after de-solvating Forms B or G by heating them in a TGA instrument to 230-250° C. Form F was characterized by techniques including XRPD, DSC, and ¹H-NMR. Table 36a summarizes some of these results.

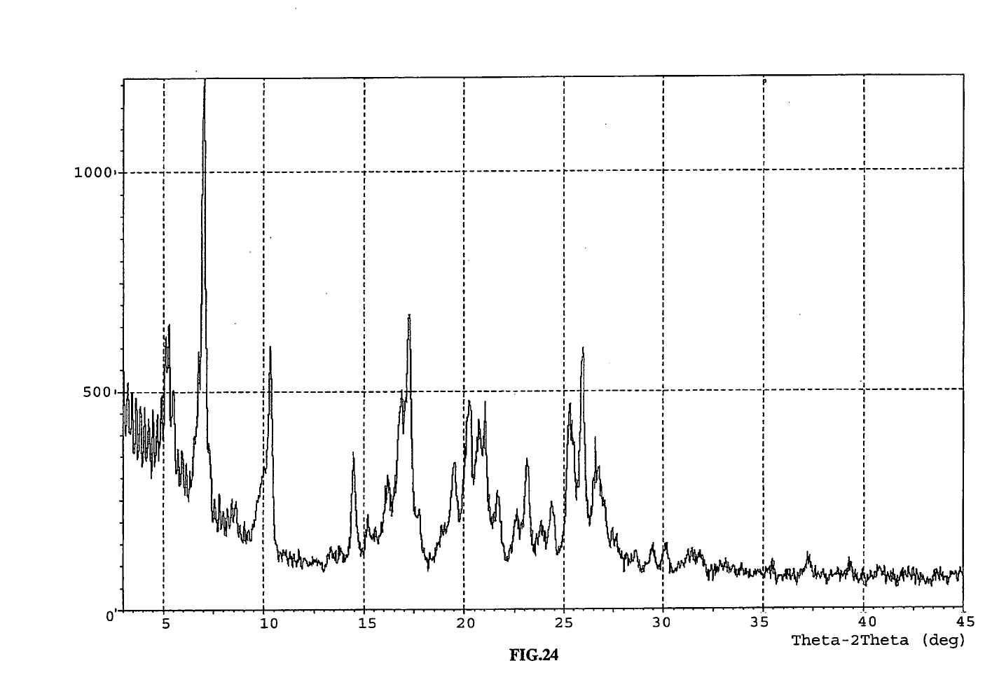

FIG. 24 shows a characteristic XRPD spectrum (CuKα, λ=1.5418 Å) of Form F. The XRPD pattern confirms that Form F is crystalline. Major X-Ray diffraction lines expressed in °2θ and their relative intensities are summarized in Table 6.

TABLE 6
Characteristic XRPD Peaks (CuKα) of Form F

| Peak No. | 2θ (°) | d-spacing | Intensity | I/Iₒ |
|---|---|---|---|---|
| 1 | 3.2450 | 27.20545 | 41 | 158 |
| 2 | 4.2764 | 20.64596 | 56 | 359 |
| 3 | 5.1988 | 16.98472 | 234 | 2476 |
| 4 | 5.5200 | 15.99711 | 130 | 596 |
| 5 | 6.4800 | 13.62916 | 61 | 129 |
| 6 | 7.0130 | 12.59447 | 755 | 5647 |
| 7 | 7.3200 | 12.06693 | 106 | 630 |
| 8 | 8.6057 | 10.26679 | 48 | 322 |
| 9 | 9.6000 | 9.20554 | 47 | 367 |
| 10 | 9.9600 | 8.87361 | 133 | 1450 |
| 11 | 10.3084 | 8.57447 | 347 | 2173 |
| 12 | 13.3040 | 6.64977 | 24 | 345 |
| 13 | 14.4843 | 6.11043 | 156 | 979 |
| 14 | 15.1888 | 5.82856 | 39 | 297 |
| 15 | 15.8400 | 5.59038 | 23 | 119 |
| 16 | 16.2000 | 5.46695 | 110 | 1185 |
| 17 | 16.8400 | 5.26059 | 276 | 2913 |
| 18 | 17.2400 | 5.13943 | 441 | 3111 |
| 19 | 17.7200 | 5.00128 | 84 | 833 |
| 20 | 18.8800 | 4.69653 | 40 | 400 |
| 21 | 19.4883 | 4.55129 | 153 | 1491 |
| 22 | 20.1886 | 4.39496 | 269 | 2680 |
| 23 | 20.7200 | 4.28343 | 218 | 2060 |
| 24 | 21.0000 | 4.22695 | 230 | 1402 |
| 25 | 21.6475 | 4.10196 | 114 | 1120 |
| 26 | 22.5920 | 3.93256 | 72 | 634 |
| 27 | 23.1216 | 3.84367 | 166 | 1263 |
| 28 | 23.8612 | 3.72617 | 44 | 315 |
| 29 | 24.3634 | 3.65049 | 80 | 566 |
| 30 | 25.2400 | 3.52566 | 255 | 1302 |
| 31 | 25.4400 | 3.49839 | 191 | 1576 |
| 32 | 25.9281 | 3.43363 | 370 | 2674 |
| 33 | 26.5600 | 3.35336 | 161 | 1517 |
| 34 | 26.7600 | 3.32875 | 140 | 0 |
| 35 | 27.0800 | 3.29013 | 77 | 1043 |
| 36 | 27.4450 | 3.24720 | 40 | 168 |
| 37 | 27.6339 | 3.22543 | 33 | 109 |
| 38 | 28.6027 | 3.11835 | 27 | 102 |
| 39 | 29.4300 | 3.03254 | 41 | 291 |
| 40 | 30.0671 | 2.96972 | 44 | 333 |
| 41 | 31.1600 | 2.86801 | 28 | 139 |
| 42 | 31.4666 | 2.84076 | 31 | 242 |
| 43 | 31.8143 | 2.81050 | 28 | 200 |
| 44 | 35.4395 | 2.53087 | 24 | 151 |
| 45 | 37.2440 | 2.41229 | 40 | 506 |
| 46 | 39.3022 | 2.29057 | 32 | 222 |
| 47 | 42.4711 | 2.12671 | 30 | 174 |

This unique set of XRPD peak positions or a subset thereof can be used to identify Form F. One such subset comprises peaks at about 7.0, 17.2, and 25.9°2θ. Another subset comprises peaks at about 5.2, 10.3 and 20.2°2θ.

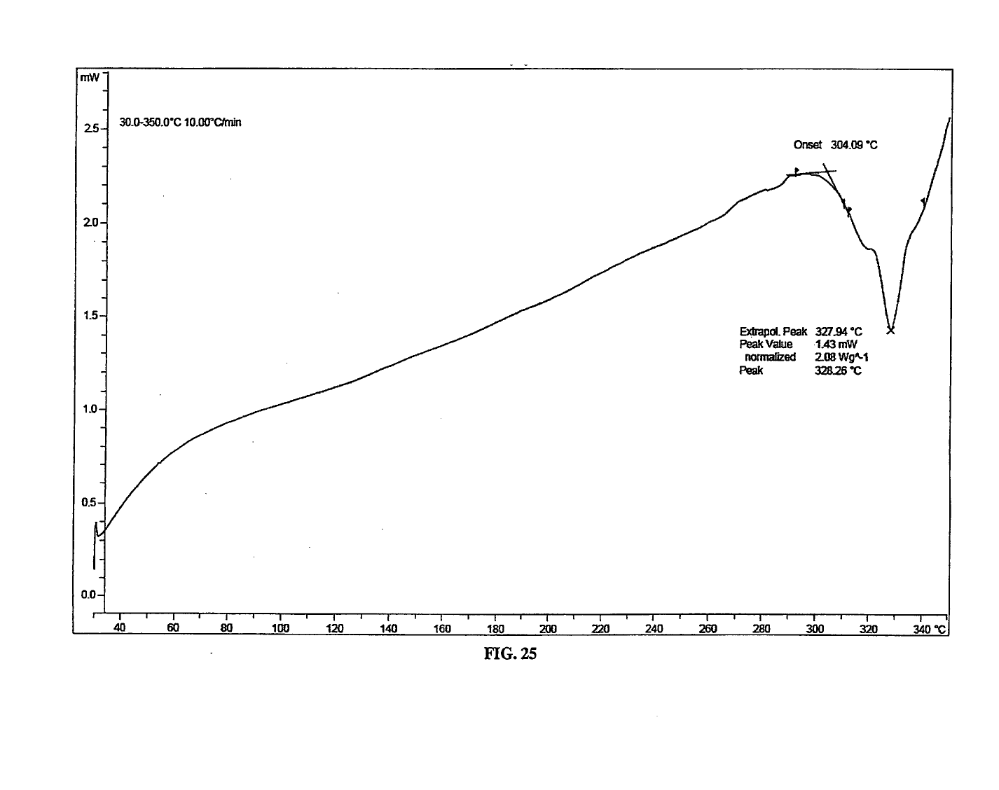

FIG. 25 shows a characteristic DSC thermogram of Form F. An endotherm was observed to onset at about 304° C. and centered from about 323° C. to about 333° C.; peak maximum is at about 328° C.

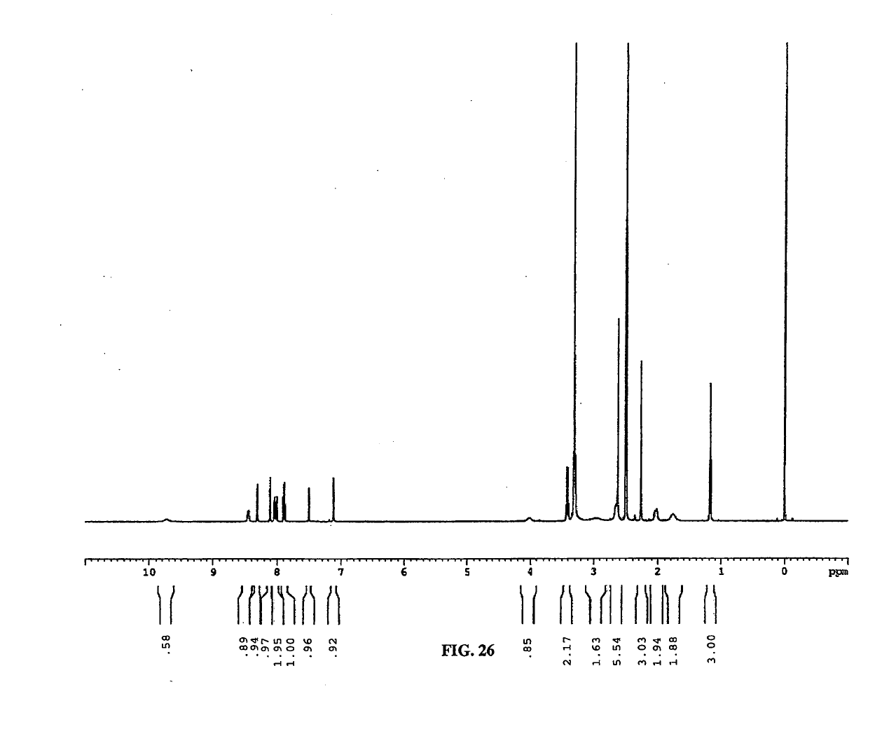

Form F was further characterized by solution ¹H NMR. The spectrum is reported in FIG. 26. Chemical assignments were not performed; however, the spectra are consistent with the known chemical structure of Compound 1.

7. Form G

Based on the available characterization data, Form G appears to be an DMF solvate polymorphic form of Compound 1. Form G was characterized by techniques including XRPD, DSC, TGA, and ¹H-NMR. Table 36b summarizes some of these results.

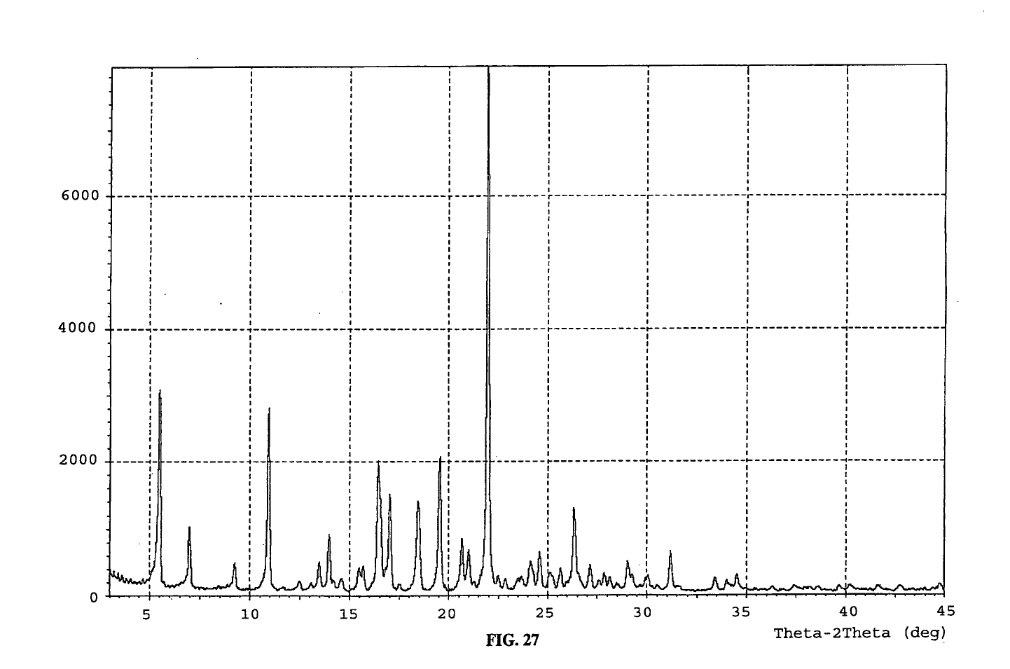

FIG. 27 shows a characteristic XRPD spectrum (CuKα, λ=1.5418 Å) of Form G. The XRPD pattern confirms that Form G is crystalline. Major X-Ray diffraction lines expressed in °2θ and their relative intensities are summarized in Table 7.

TABLE 7
Characteristic XRPD Peaks (CuKα) of Form G

| Peak No. | 2θ (°) | d-spacing | Intensity | I/Iₒ |
|---|---|---|---|---|
| 1 | 5.4847 | 16.09999 | 2359 | 10474 |
| 2 | 6.9827 | 12.64905 | 710 | 3038 |
| 3 | 9.2290 | 9.57475 | 309 | 1561 |
| 4 | 10.9344 | 8.08494 | 2101 | 8785 |
| 5 | 13.4725 | 6.56697 | 321 | 1670 |
| 6 | 13.9654 | 6.33629 | 669 | 2821 |
| 7 | 15.4400 | 5.73430 | 257 | 1234 |
| 8 | 15.6800 | 5.64706 | 280 | 1375 |
| 9 | 16.4628 | 5.38027 | 1566 | 9294 |
| 10 | 17.0020 | 5.21083 | 1161 | 5256 |
| 11 | 18.4415 | 4.80720 | 1105 | 6367 |
| 12 | 19.5223 | 4.54344 | 1678 | 7240 |
| 13 | 20.6429 | 4.29926 | 625 | 2760 |
| 14 | 20.9709 | 4.23275 | 460 | 2591 |
| 15 | 21.9517 | 4.04579 | 6414 | 24463 |
| 16 | 24.1289 | 3.68544 | 315 | 3198 |
| 17 | 24.5621 | 3.62141 | 466 | 1782 |
| 18 | 25.1330 | 3.54043 | 205 | 1452 |
| 19 | 25.6216 | 3.47401 | 228 | 1195 |
| 20 | 26.3248 | 3.38278 | 1026 | 4893 |
| 21 | 27.1244 | 3.28485 | 310 | 1289 |
| 22 | 29.0166 | 3.07480 | 381 | 2124 |
| 23 | 31.1516 | 2.86876 | 518 | 2161 |
| 24 | 34.4828 | 2.59887 | 216 | 1340 |

This unique set of XRPD peak positions or a subset thereof can be used to identify Form G. One such subset comprises peaks at about 5.5, 10.9 and 22.0 degrees °2θ. Another subset comprises peaks at about 16.5, 18.4 and 19.5°2θ.

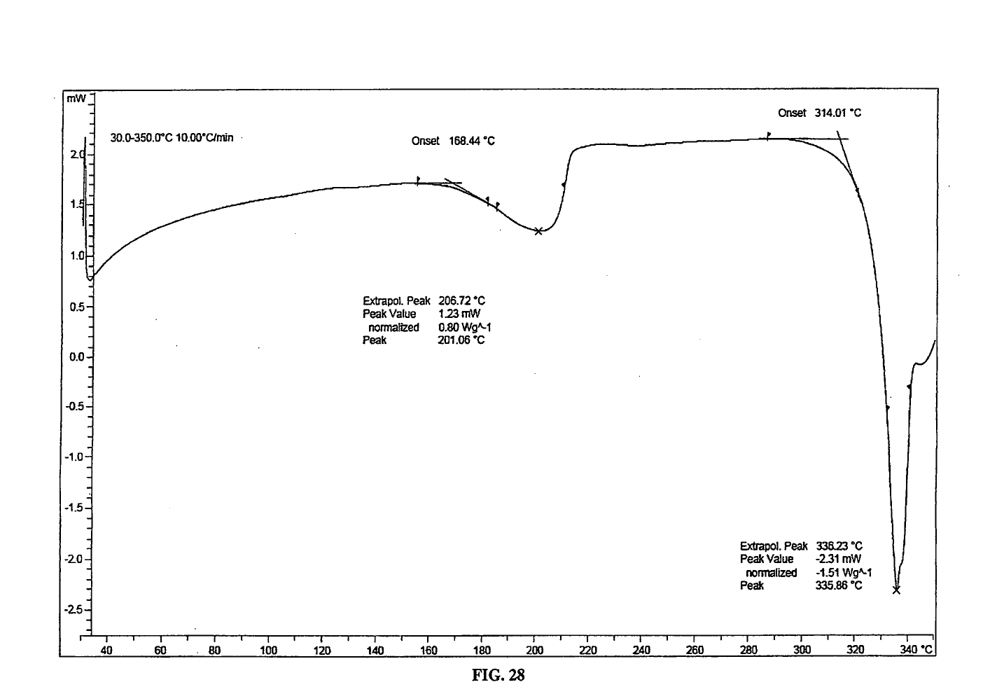

FIG. 28 shows a characteristic DSC thermogram of Form G. The thermogram shows a broad endotherm centered at about 201° C. and a second endotherm which onset at approximately 314° C. and centered from about 334° C. to about 338° C. This second endotherm peaked at approximately 336° C. (peak maximum).

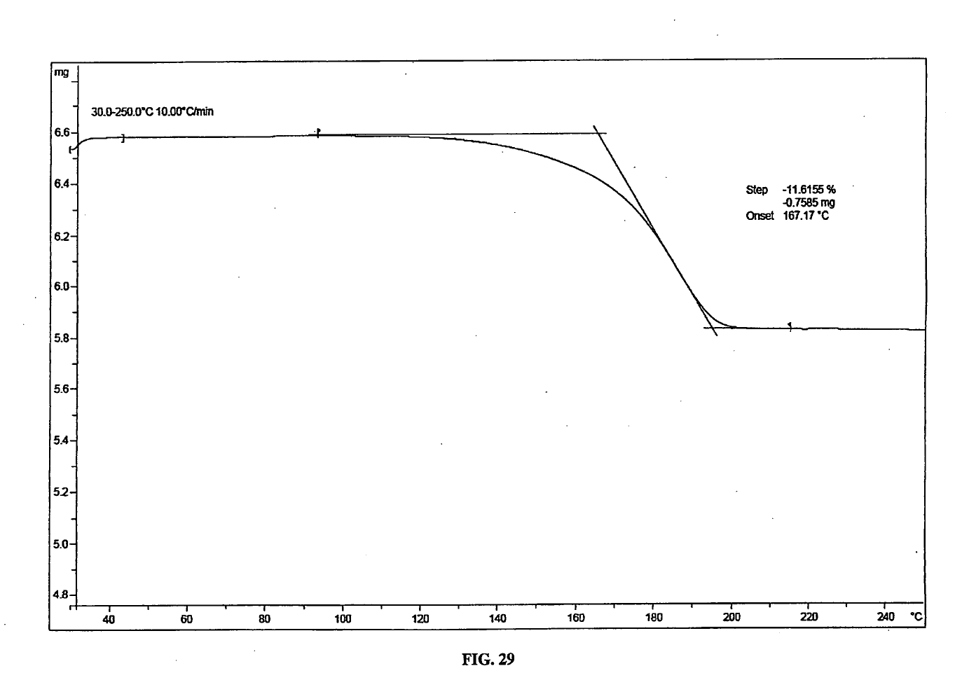

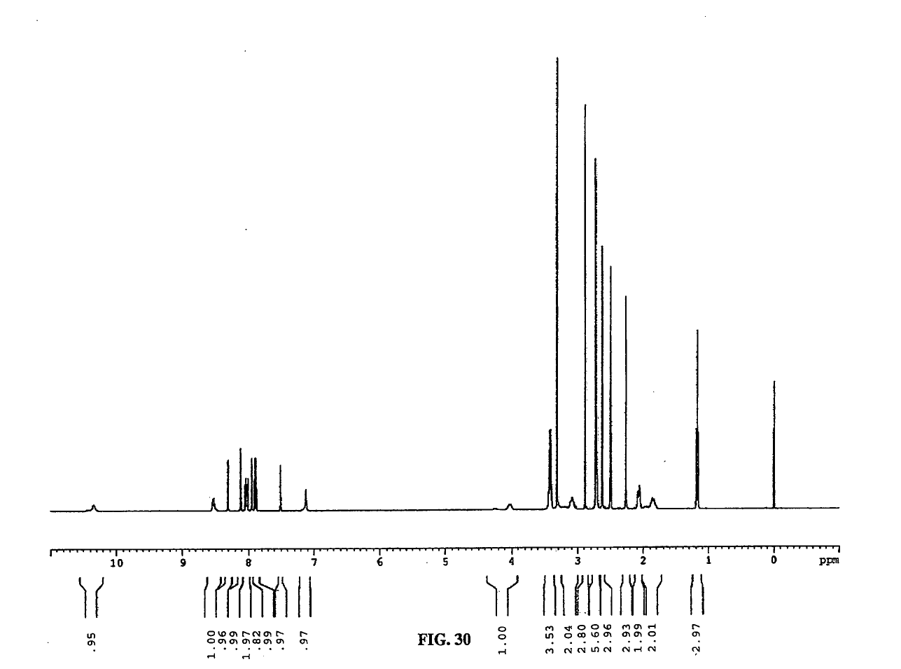

FIG. 29 is a TGA thermogram of Form G.

Form G was further characterized by solution ¹H NMR. The spectrum is reported in FIG. 30. The spectrum is consistent with one molar equivalent of solvent present, as well as the known chemical structure of Compound 1.

Further details related to the preparation and characterization of Form G are presented below in the Examples section.

8. Form I

Based on the available characterization data, Form I appears to be a THF solvate polymorphic form of Compound 1. Form I was characterized by techniques including XRPD, DSC, TGA, and ¹H-NMR. Table 36b summarizes some of these results.

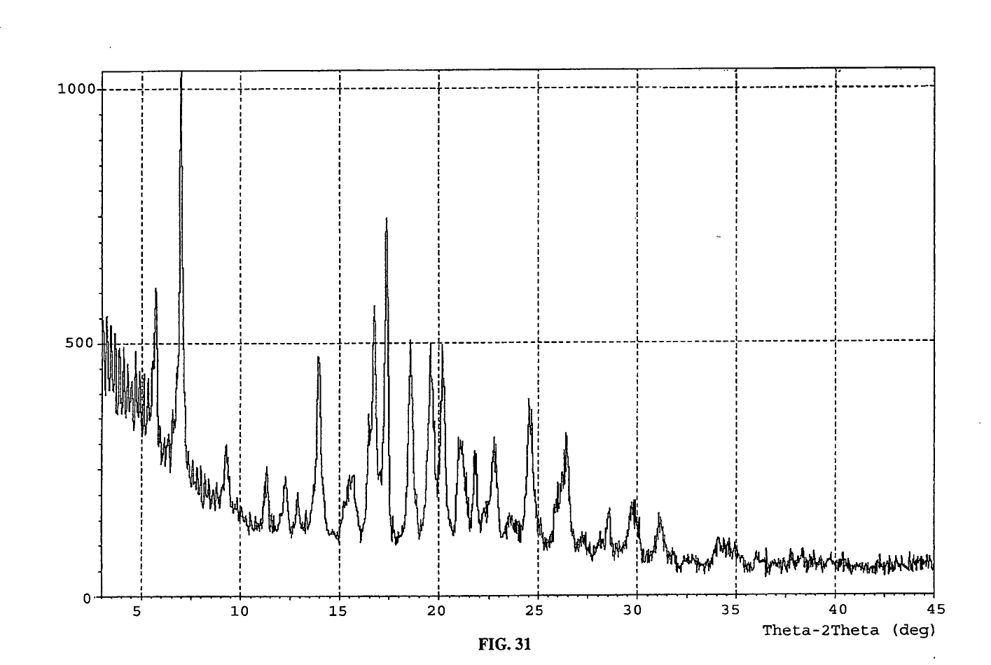

FIG. 31 shows a characteristic XRPD spectrum (CuKα, λ=1.5418 Å) of Form I. The XRPD pattern confirms that Form I is crystalline. Major X-Ray diffraction lines expressed in °2θ and their relative intensities are summarized in Table 8.

TABLE 8
Characteristic XRPD Peaks (CuKα) of Form I

| Peak No. | 2θ (°) | d-spacing | Intensity | I/Iₒ |
|---|---|---|---|---|
| 1 | 3.8726 | 22.79778 | 37 | 165 |
| 2 | 4.0783 | 21.64836 | 38 | 96 |
| 3 | 4.2951 | 20.55611 | 27 | 131 |
| 4 | 4.7018 | 18.77890 | 41 | 313 |
| 5 | 5.3200 | 16.59804 | 49 | 269 |
| 6 | 5.6645 | 15.58934 | 216 | 1319 |
| 7 | 6.3279 | 13.95641 | 33 | 96 |
| 8 | 6.5600 | 13.46313 | 67 | 326 |
| 9 | 6.9855 | 12.64399 | 564 | 3563 |
| 10 | 7.5900 | 11.63827 | 23 | 88 |
| 11 | 7.7960 | 11.33120 | 18 | 86 |
| 12 | 9.0400 | 9.77450 | 23 | 45 |
| 13 | 9.2887 | 9.51335 | 90 | 643 |
| 14 | 11.3004 | 7.82389 | 77 | 465 |
| 15 | 12.0000 | 7.36928 | 17 | 47 |
| 16 | 12.2694 | 7.20808 | 79 | 478 |
| 17 | 12.8880 | 6.86346 | 50 | 267 |
| 18 | 13.6000 | 6.50569 | 35 | 323 |
| 19 | 13.9480 | 6.34415 | 281 | 1624 |
| 20 | 14.2400 | 6.21471 | 45 | 314 |
| 21 | 15.2400 | 5.80910 | 49 | 579 |
| 22 | 15.4800 | 5.71957 | 78 | 0 |
| 23 | 15.6400 | 5.66141 | 92 | 670 |
| 24 | 15.8800 | 5.57639 | 33 | 156 |
| 25 | 16.4400 | 5.38768 | 136 | 563 |
| 26 | 16.7316 | 5.29443 | 345 | 2743 |
| 27 | 17.3561 | 5.10531 | 481 | 3013 |
| 28 | 17.7200 | 5.00128 | 18 | 88 |
| 29 | 18.5670 | 4.77499 | 273 | 1927 |
| 30 | 18.8800 | 4.69653 | 34 | 219 |
| 31 | 19.5848 | 4.52908 | 261 | 2408 |
| 32 | 20.1598 | 4.40118 | 260 | 2026 |
| 33 | 21.0841 | 4.21028 | 140 | 1329 |
| 34 | 21.3600 | 4.15651 | 55 | 266 |
| 35 | 21.7815 | 4.07702 | 121 | 674 |
| 36 | 22.2000 | 4.00110 | 38 | 167 |
| 37 | 22.7480 | 3.90594 | 135 | 1389 |
| 38 | 23.5760 | 3.77060 | 35 | 392 |
| 39 | 24.2800 | 3.66284 | 43 | 269 |
| 40 | 24.5571 | 3.62214 | 196 | 1801 |
| 41 | 25.1200 | 3.54223 | 26 | 141 |
| 42 | 25.8800 | 3.43991 | 64 | 778 |
| 43 | 26.1600 | 3.40372 | 107 | 0 |
| 44 | 26.4000 | 3.37332 | 158 | 1391 |
| 45 | 26.7600 | 3.32875 | 18 | 106 |
| 46 | 27.2628 | 3.26849 | 21 | 171 |
| 47 | 28.1166 | 3.17115 | 22 | 158 |

This unique set of XRPD peak positions or a subset thereof can be used to identify Form I. One such subset comprises peaks at about 7.0, 16.7 and 17.4°2θ. Another subset comprises peaks at about 19.6, 20.2 and 24.6°2θ.

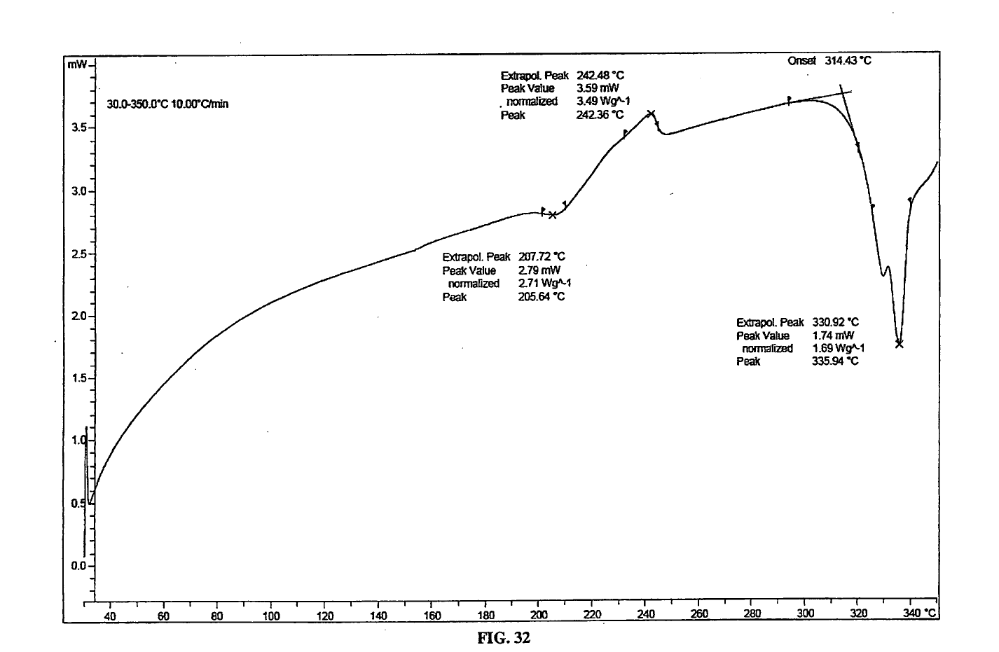

FIG. 32 shows a characteristic DSC thermogram of Form I, showing multiple events, with an endotherm observed near the temperature range observed for bound weight loss by TGA and followed by an exothermic event consistent with re-crystallization to an anhydrous form. The first endothermic event was centered at about 206° C. The exothermic event was centered at about 242° C. The second endothermic event onset at about 314° C. and centered from about 320° C. to about 340° C. and peak maximum centered at approximately 336° C.

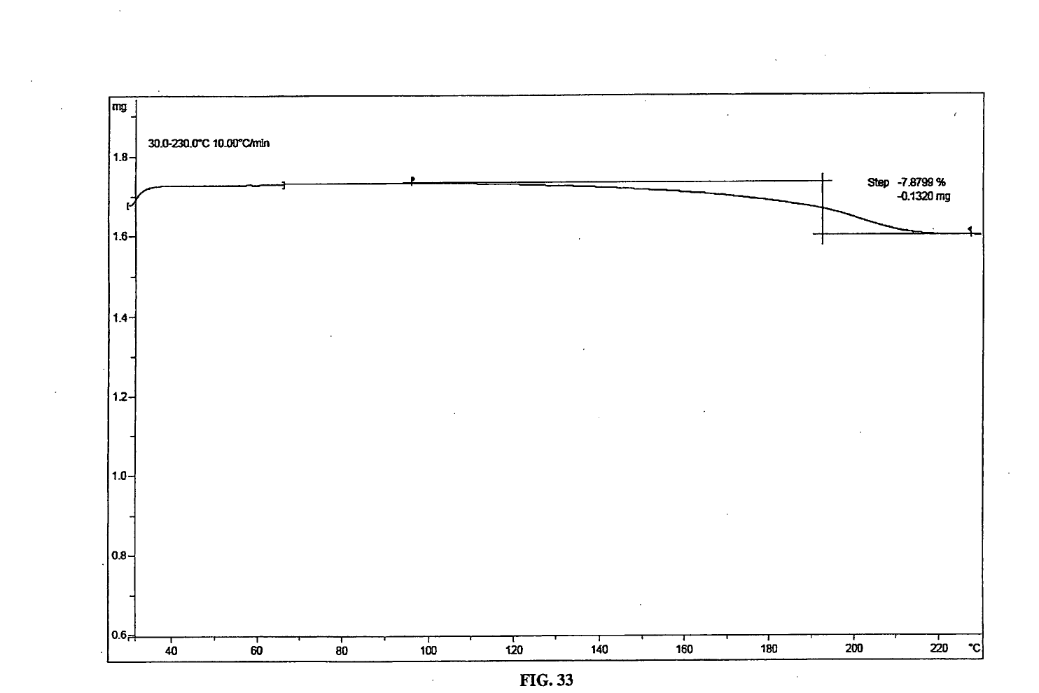

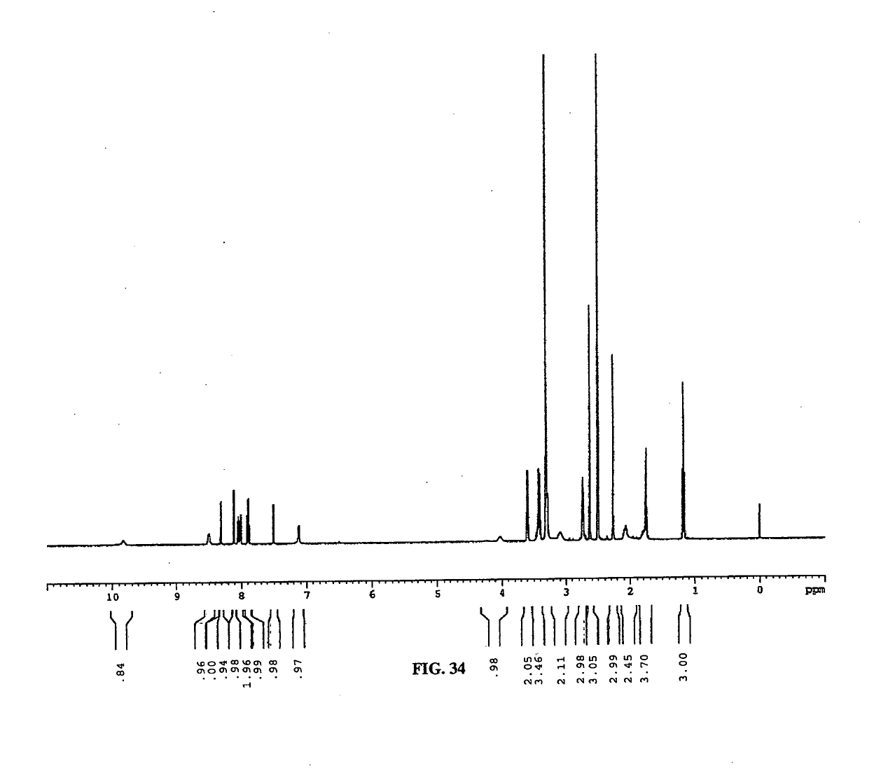

FIG. 33 is a TGA thermogram of Form I.

Form I was further characterized by solution ¹H NMR. The spectrum is reported in FIG. 34. The spectrum is consistent with one half molar equivalent of THF present, as well as the known chemical structure of Compound 1.

Further details related to the preparation and characterization of Form I are presented below in the Examples section.

9. Form J

Based on the available characterization data, Form J appears to be an anhydrous polymorphic form of Compound 1. Form J was characterized by techniques including XRPD, DSC, TGA, and ¹H-NMR. Table 36a summarizes some of these results.

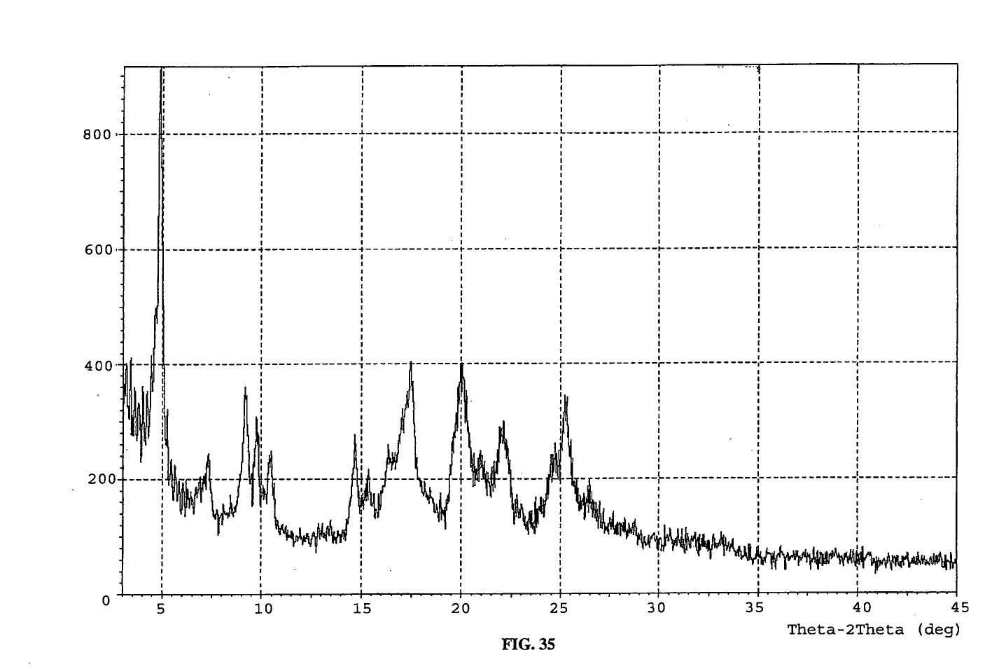

FIG. 35 shows a characteristic XRPD spectrum (CuKα, λ=1.5418 Å) of Form J. The XRPD pattern confirms that Form J is crystalline. Major X-Ray diffraction lines expressed in °2θ and their relative intensities are summarized in Table 9.

TABLE 9
Characteristic XRPD Peaks (CuKα) of Form J

| Peak No. | 2θ (°) | d-spacing | Intensity | I/Iₒ |
|---|---|---|---|---|
| 1 | 4.1200 | 21.42934 | 15 | 80 |
| 2 | 4.4800 | 19.70812 | 90 | 736 |
| 3 | 4.8696 | 18.13218 | 491 | 3649 |
| 4 | 5.2400 | 16.85127 | 31 | 231 |
| 5 | 6.8000 | 12.98849 | 28 | 173 |
| 6 | 7.2697 | 12.15031 | 69 | 675 |
| 7 | 8.4600 | 10.44328 | 19 | 208 |
| 8 | 8.8800 | 9.95026 | 49 | 381 |
| 9 | 9.1910 | 9.61425 | 173 | 1428 |
| 10 | 9.7334 | 9.07968 | 131 | 1178 |
| 11 | 10.4330 | 8.47234 | 103 | 1118 |
| 12 | 13.4420 | 6.58181 | 16 | 146 |
| 13 | 14.6589 | 6.03804 | 109 | 1062 |
| 14 | 15.3600 | 5.76398 | 57 | 694 |
| 15 | 16.0400 | 5.52112 | 37 | 707 |
| 16 | 16.3600 | 5.41384 | 86 | 0 |
| 17 | 16.9600 | 5.22364 | 124 | 0 |
| 18 | 17.4800 | 5.06940 | 197 | 3351 |
| 19 | 18.1600 | 4.88108 | 29 | 0 |
| 20 | 18.4800 | 4.79728 | 22 | 241 |
| 21 | 19.6400 | 4.51647 | 113 | 931 |
| 22 | 20.0400 | 4.42722 | 199 | 2536 |
| 23 | 20.9600 | 4.23492 | 79 | 1497 |
| 24 | 22.0616 | 4.02589 | 123 | 2087 |
| 25 | 22.7855 | 3.89960 | 28 | 106 |
| 26 | 23.0022 | 3.86335 | 23 | 167 |
| 27 | 24.0000 | 3.70494 | 29 | 199 |
| 28 | 24.7200 | 3.59863 | 95 | 1539 |
| 29 | 25.2440 | 3.52511 | 167 | 2054 |
| 30 | 25.8000 | 3.45039 | 44 | 0 |
| 31 | 26.1200 | 3.40884 | 32 | 0 |
| 32 | 26.3960 | 3.37382 | 46 | 673 |
| 33 | 28.2075 | 3.16113 | 15 | 69 |
| 34 | 28.7378 | 3.10399 | 17 | 143 |
| 35 | 29.7000 | 3.00559 | 15 | 89 |
| 36 | 30.5186 | 2.92681 | 17 | 112 |
| 37 | 31.7350 | 2.81734 | 15 | 71 |
| 38 | 32.7628 | 2.73127 | 17 | 39 |
| 39 | 33.1700 | 2.69866 | 16 | 228 |
| 40 | 40.5466 | 2.22310 | 16 | 112 |

This unique set of XRPD peak positions or a subset thereof can be used to identify Form J. One such subset comprises peaks at about 4.9, 17.5 and 20.0°2θ. Another subset comprises peaks at about 9.2, 22.1 and 25.2°2θ.

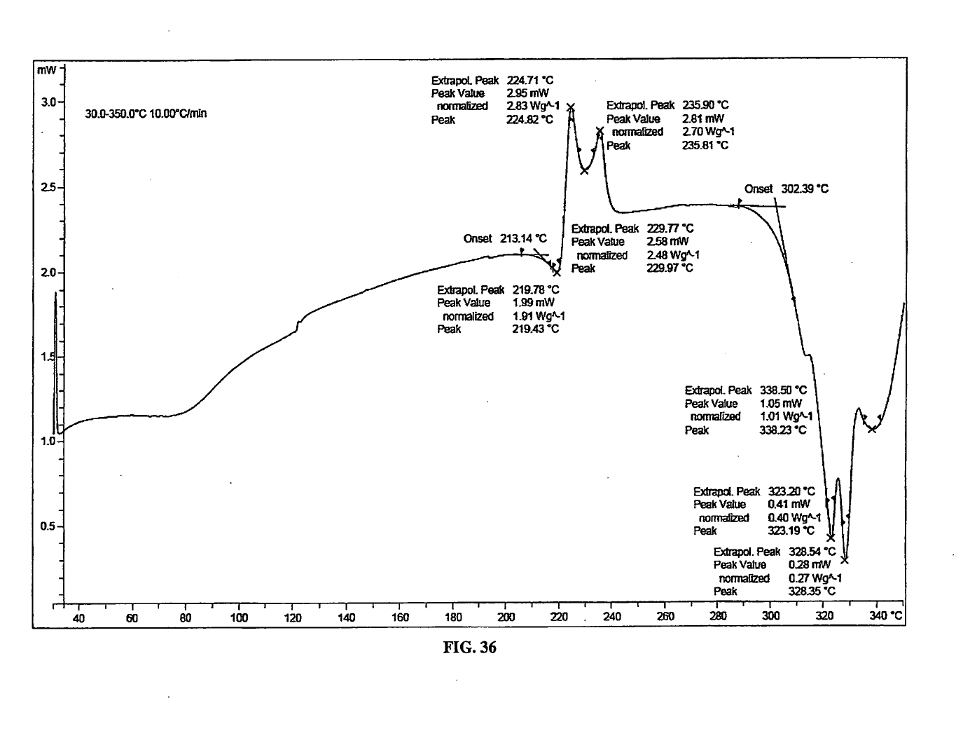

FIG. 36 shows a characteristic DSC thermogram of Form J. The thermogram shows a first endotherm centered at about 219° C., a forked exotherm with peaks centered at about 223° C. and 236° C., followed by a forked endotherm which onset at 302° C. with peaks centered at approximately 323° C., 328° C. and 338° C.

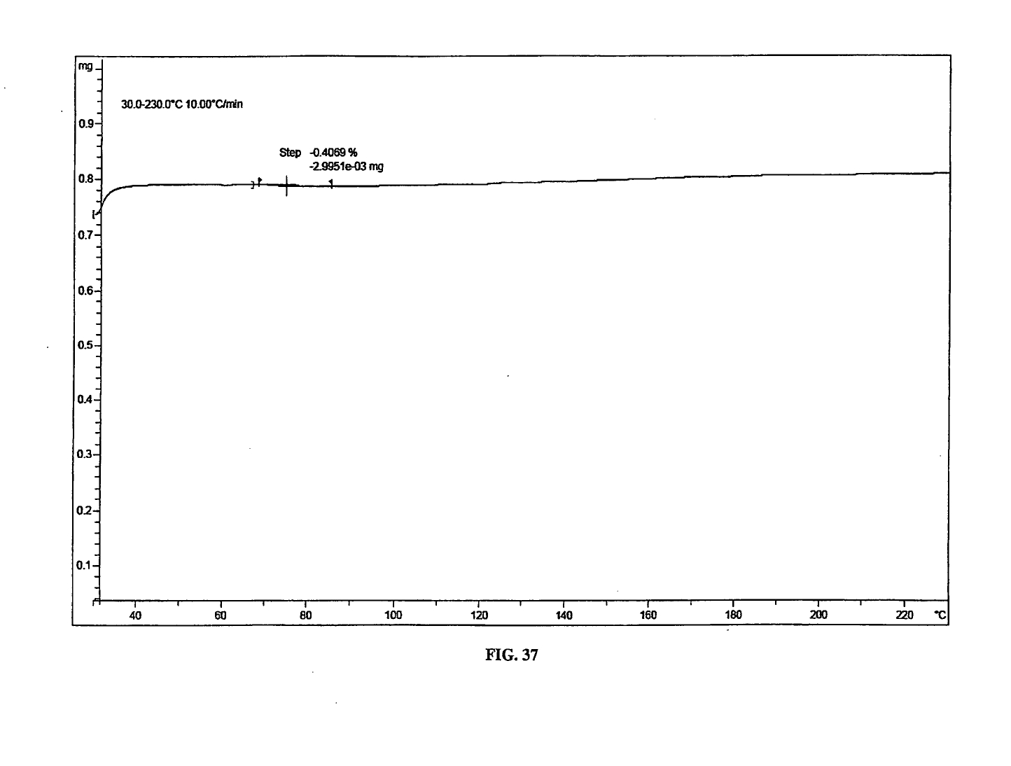

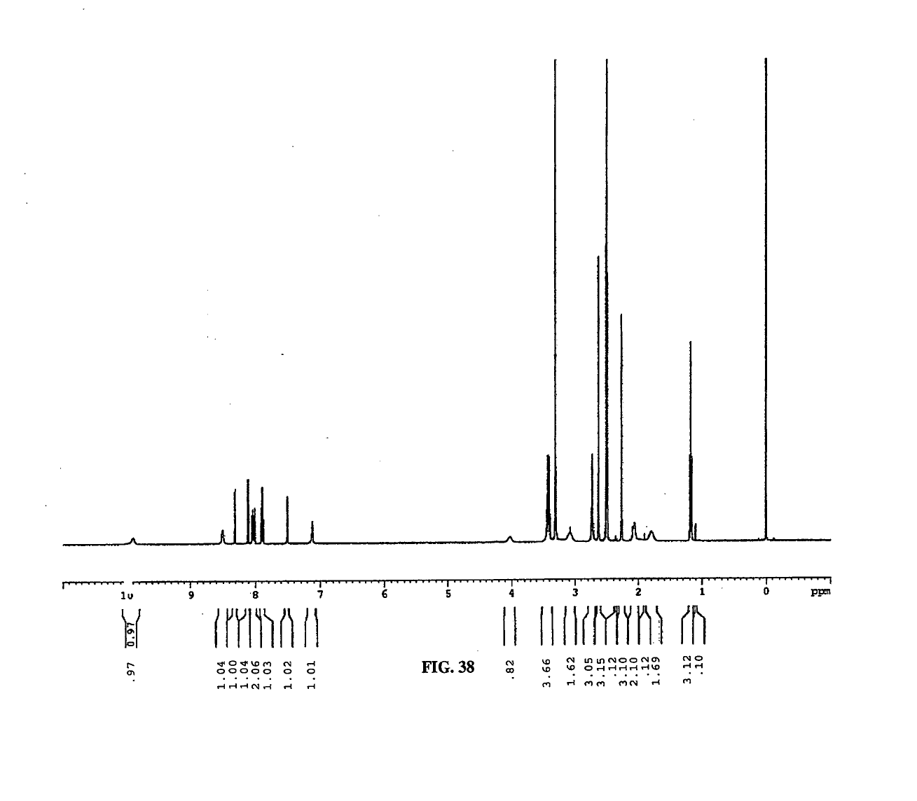

FIG. 37 is a TGA thermogram of Form J. TGA analysis showed no weight loss or only small weight losses likely due to residual solvents.

Form J was further characterized by solution ¹H NMR. The spectrum is reported in FIG. 38. Chemical assignments were not performed; however, the spectra are consistent with the known chemical structure of Compound 1.

Further details related to the preparation and characterization of Form J are presented below in the Examples section.

10. Form K

Based on the available characterization data, Form K appears to be an anhydrous polymorphic form of Compound 1. Form K was characterized by techniques including XRPD, DSC, TGA, and ¹H-NMR. Table 36a summarizes some of these results.

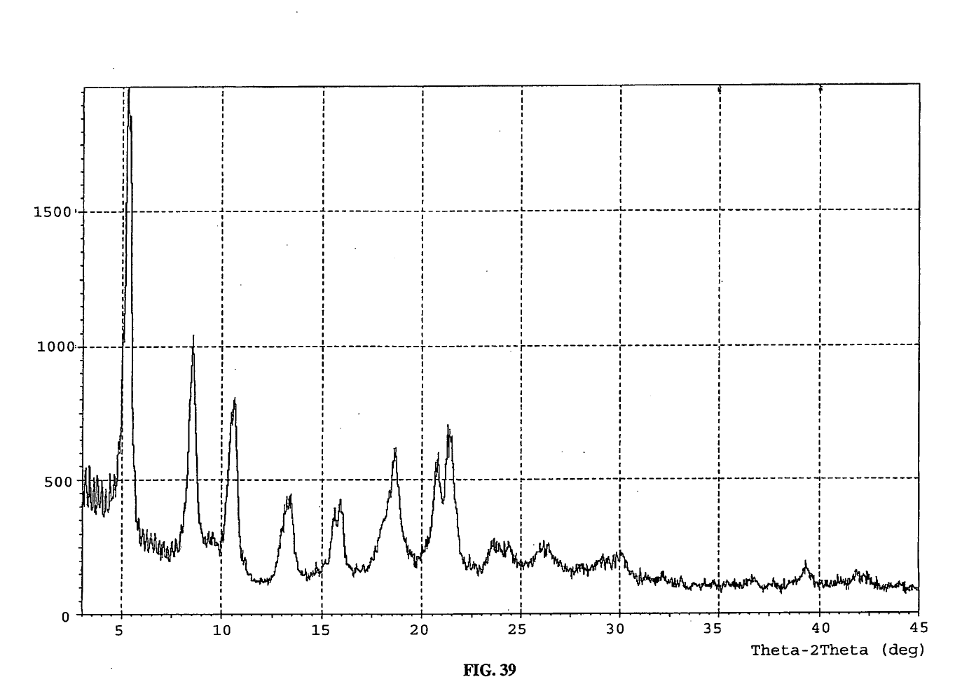

FIG. 39 shows a characteristic XRPD spectrum (CuKα, λ=1.5418 Å) of Form K. The XRPD pattern confirms that Form K is crystalline. Major X-Ray diffraction lines expressed in °2θ and their relative intensities are summarized in Table 10.

TABLE 10
Characteristic XRPD Peaks (CuKα) of Form K

| Peak No. | 2θ (°) | d-spacing | Intensity | I/Iₒ |
|---|---|---|---|---|
| 1 | 4.5600 | 19.36254 | 69 | 1117 |
| 2 | 5.2901 | 16.69179 | 1302 | 14236 |
| 3 | 8.0800 | 10.93355 | 102 | 765 |
| 4 | 8.5269 | 10.36149 | 606 | 7757 |
| 5 | 9.4800 | 9.32180 | 76 | 0 |
| 6 | 10.5407 | 8.38602 | 502 | 8225 |
| 7 | 13.2675 | 6.66798 | 231 | 4157 |
| 8 | 15.5200 | 5.70492 | 145 | 1273 |
| 9 | 15.8800 | 5.57639 | 196 | 2279 |
| 10 | 17.9600 | 4.93498 | 122 | 1574 |
| 11 | 18.5502 | 4.77928 | 329 | 5144 |
| 12 | 19.4400 | 4.56248 | 39 | 317 |
| 13 | 20.0400 | 4.42722 | 45 | 666 |
| 14 | 20.7200 | 4.28343 | 29B | 3200 |
| 15 | 21.3381 | 4.16073 | 396 | 6209 |
| 16 | 23.5200 | 3.77945 | 75 | 977 |
| 17 | 23.8800 | 3.72328 | 63 | 0 |
| 18 | 24.4000 | 3.64510 | 71 | 1089 |
| 19 | 26.0000 | 3.42430 | 61 | 975 |
| 20 | 26.4000 | 3.37332 | 64 | 900 |
| 21 | 29.0400 | 3.07238 | 45 | 953 |
| 22 | 29.4000 | 3.03557 | 44 | 0 |
| 23 | 29.7600 | 2.99966 | 53 | 581 |
| 24 | 30.0800 | 2.96848 | 67 | 711 |
| 25 | 39.2819 | 2.29171 | 57 | 897 |
| 26 | 41.8467 | 2.15699 | 41 | 736 |

This unique set of XRPD peak positions or a subset thereof can be used to identify Form K. One such subset comprises peaks at about 5.3, 8.5 and 10.5°2θ. Another subset comprises peaks at about 13.3, 18.6 and 21.3°2θ.

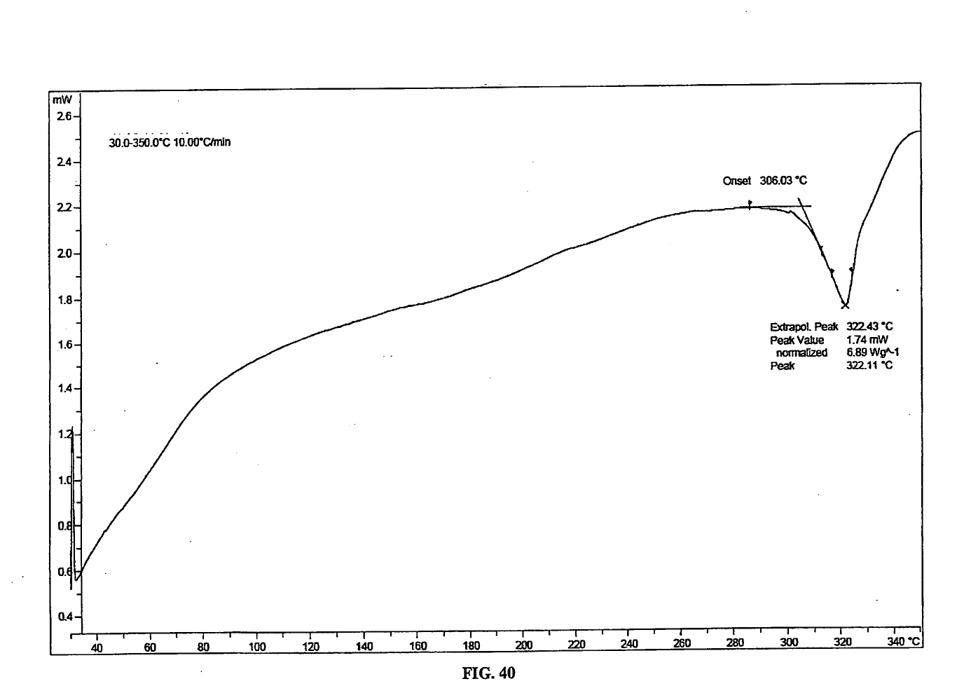

FIG. 40 shows a characteristic DSC thermogram of Form K. An endotherm which onset at about 306° C. and centered at about 322° C. (peak maximum) was observed.

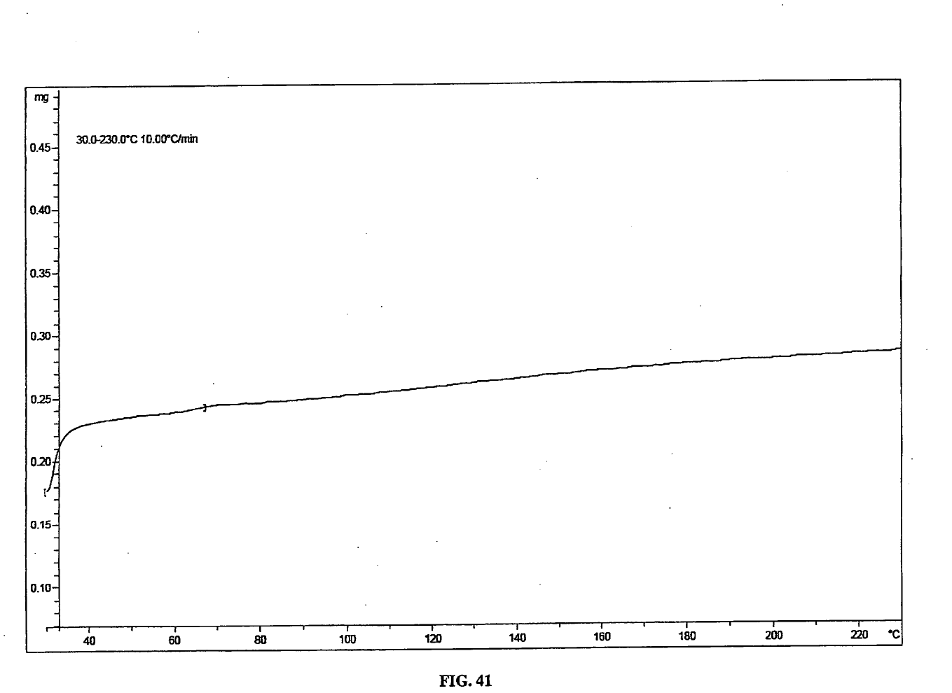

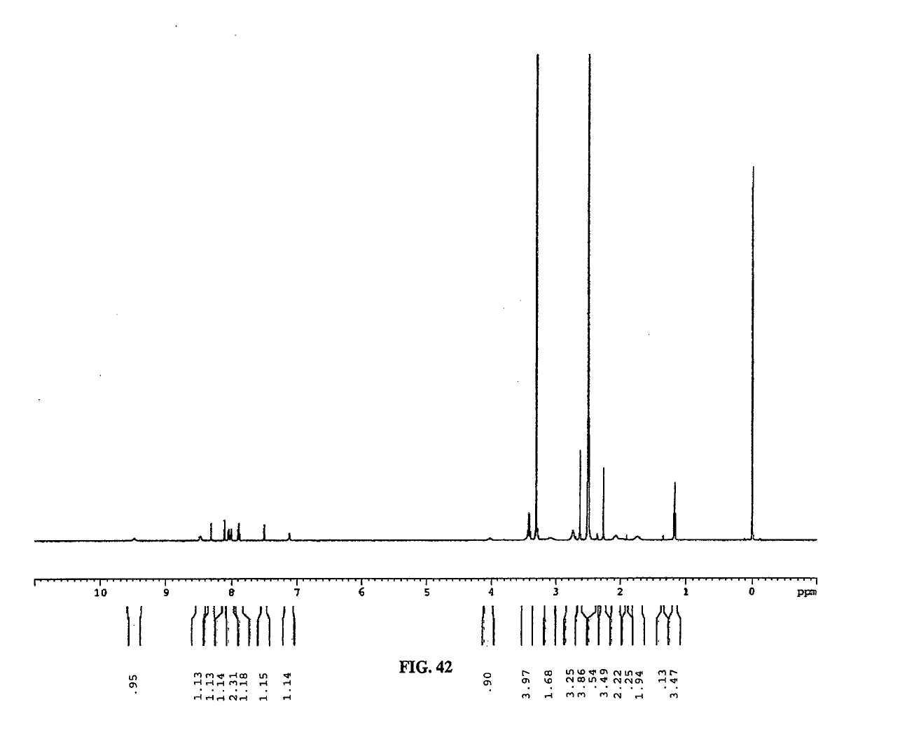

FIG. 41 is a TGA thermogram of Form K. TGA analysis showed no weight loss or only small weight losses likely due to residual solvents.

Form K was further characterized by solution ¹H NMR. The spectrum is reported in FIG. 42. Chemical assignments were not performed; however, the spectra are consistent with the known chemical structure of Compound 1.

Further details related to the preparation and characterization of Form K are presented below in the Examples section.

11. Form L

Based on the available characterization data, Form L appears to be a channel hydrate polymorphic form of Compound 1 that is stable at ambient conditions. Form L was characterized by techniques including XRPD, DSC, TGA, ¹H-NMR and moisture sorption analysis. Table 36a summarizes some of these results.

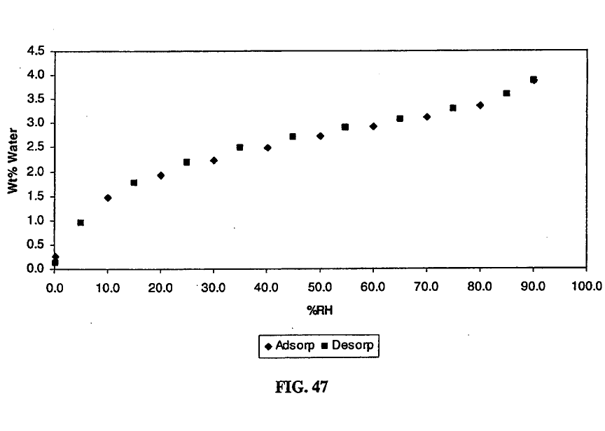

Form L is consistent with a channel hydrate based on KF and moisture sorption data (FIG. 47). KF analysis showed 2.9% water where 3.2% is theoretical for a monohydrate. The moisture sorption curve showed Form L to be moderately hygroscopic with a maximum water uptake of 3.9% at 90% RH. The shape of the curve is consistent with water able to be freely bound/removed based on temperature and relative humidity without significantly affecting the unit cell (i.e. form). The experiment did not time out (>4 hours) at any point and hysteresis was not observed upon desorption. Slurry experiments showed Form L to convert to the monohydrate Form A in water and to the anhydrate Form C in all other solvents. This is consistent with Form C being more thermodynamically stable than Form L at ambient temperature in non-aqueous environments.

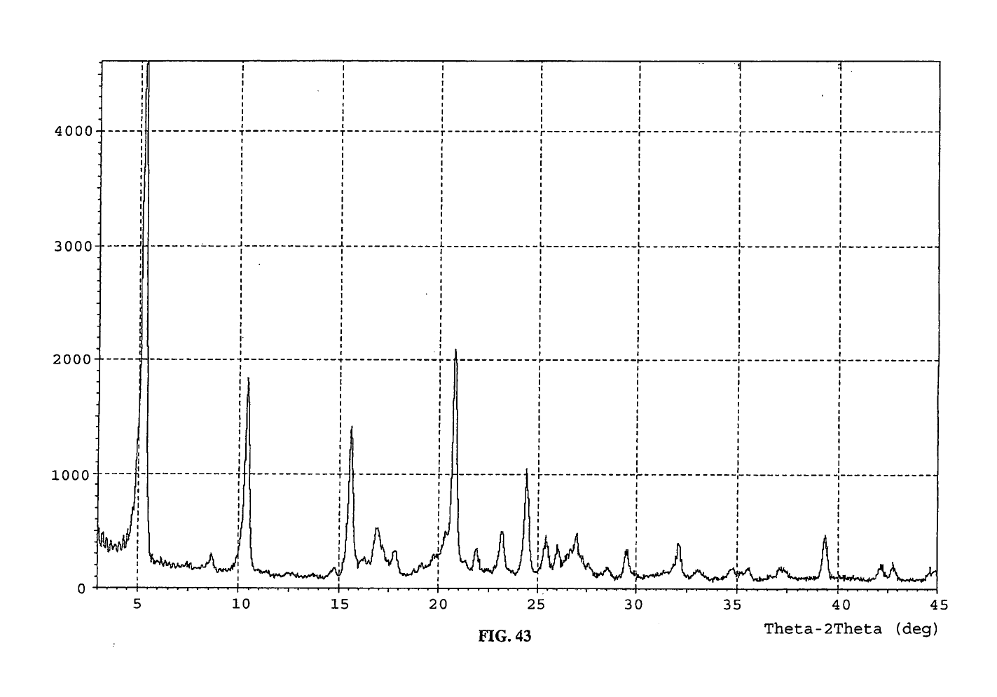

FIG. 43 shows a characteristic XRPD spectrum (CuKα, λ=1.5418 Å) of Form L. XRPD analysis which showed the same pattern before and after drying at 80° C. for one hour. The XRPD pattern confirms that Form L is crystalline. Major X-Ray diffraction lines expressed in °2θ and their relative intensities are summarized in Table 11.

TABLE 11
Characteristic XRPD Peaks (CuKα) of Form L

| Peak No. | 2θ (°) | d-spacing | Intensity | I/Iₒ |
|---|---|---|---|---|
| 1 | 4.6800 | 18.86633 | 216 | 3765 |
| 2 | 5.2436 | 16.83971 | 3437 | 28787 |
| 3 | 10.3883 | 8.50870 | 1268 | 10874 |
| 4 | 15.5487 | 5.69445 | 945 | 8156 |
| 5 | 16.2000 | 5.46695 | 103 | 0 |
| 6 | 16.8800 | 5.24822 | 317 | 3104 |
| 7 | 17.2000 | 5.15129 | 161 | 2040 |
| 8 | 17.7805 | 4.98440 | 165 | 1404 |
| 9 | 19.7600 | 4.48931 | 109 | 1601 |
| 10 | 20.2400 | 4.38392 | 227 | 3043 |
| 11 | 20.7515 | 4.27700 | 1547 | 11336 |
| 12 | 21.8685 | 4.06100 | 146 | 1064 |
| 13 | 23.1344 | 3.84157 | 290 | 2388 |
| 14 | 24.3913 | 3.64638 | 667 | 5099 |
| 15 | 25.3923 | 3.50486 | 224 | 1910 |
| 16 | 25.9912 | 3.42544 | 174 | 1251 |
| 17 | 26.6000 | 3.34841 | 155 | 1724 |
| 18 | 26.9200 | 3.30933 | 262 | 1535 |
| 19 | 27.2400 | 3.27117 | 104 | 1360 |
| 20 | 29.4781 | 3.02770 | 190 | 1539 |
| 21 | 32.0605 | 2.78948 | 237 | 2340 |
| 22 | 39.3236 | 2.28937 | 287 | 2215 |
| 23 | 42.7408 | 2.11391 | 107 | 1358 |

This unique set of XRPD peak positions or a subset thereof can be used to identify Form L. One such subset comprises peaks at about 5.2, 10.4 and 20.7°2θ. Another subset comprises peaks at about 15.5, 16.9 and 24.4°2θ.

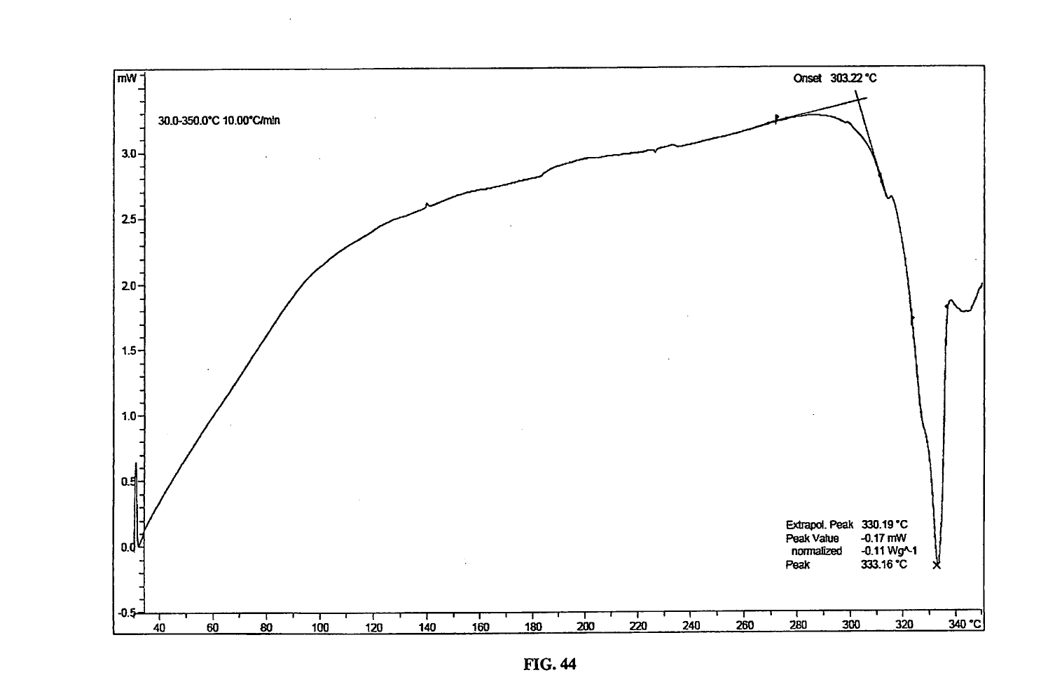

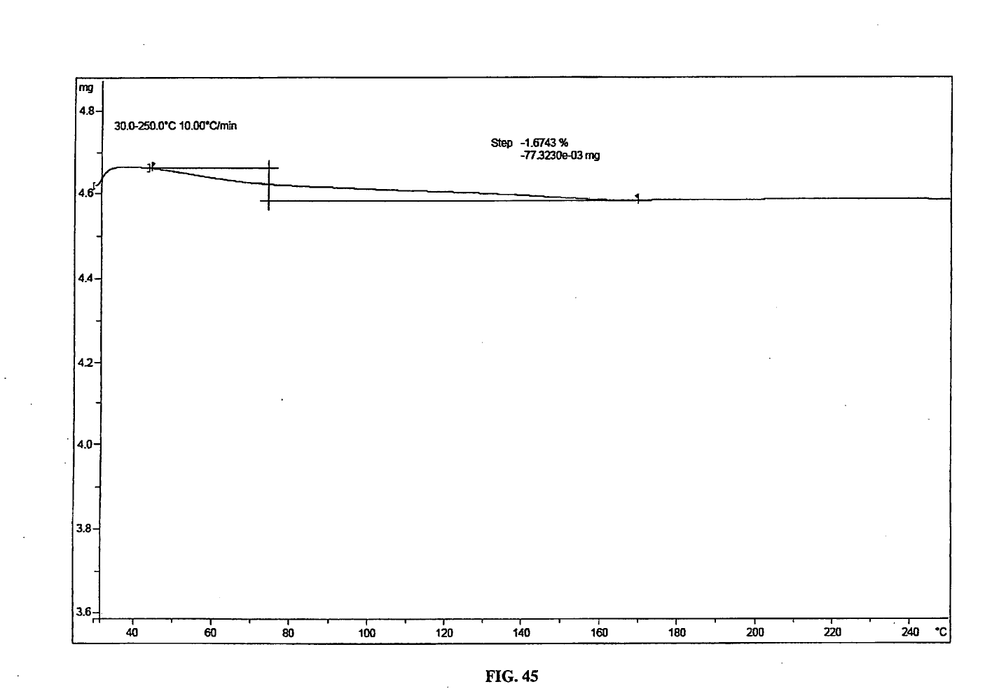

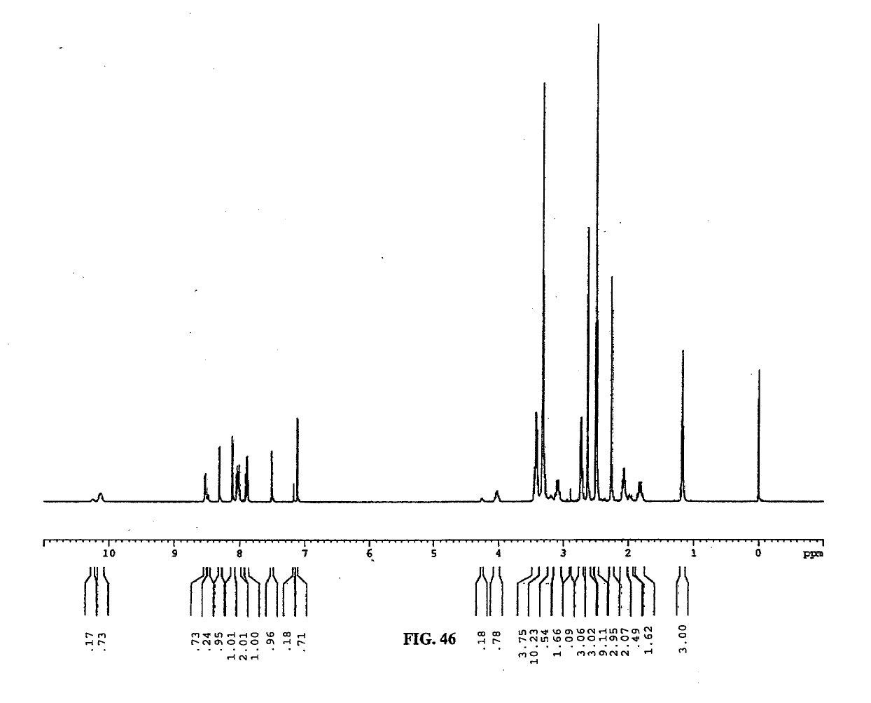

FIG. 44 shows a characteristic DSC thermogram of Form L. An endotherm which onset at about 303° C. and centered at approximately 333° C. (peak maximum) was observed. FIG. 45 is a TGA thermogram of Form L, showing a weight loss of approximately 1.7% at a temperature below 100° C. The theoretical weight loss for a monohydrate is 3.2%.

Form L was further characterized by solution ¹H NMR. The spectrum is reported in FIG. 46. Chemical assignments were not performed; however, the spectra are consistent with the known chemical structure of Compound 1.

Further details related to the preparation and characterization of Form L are presented below in the Examples section.

12. Form M

Based on the available characterization data, Form M appears to be an hydrate polymorphic form of Compound 1. Form M was characterized by techniques including XRPD, DSC, TGA, and ¹H-NMR. Table 36a summarizes some of these results.

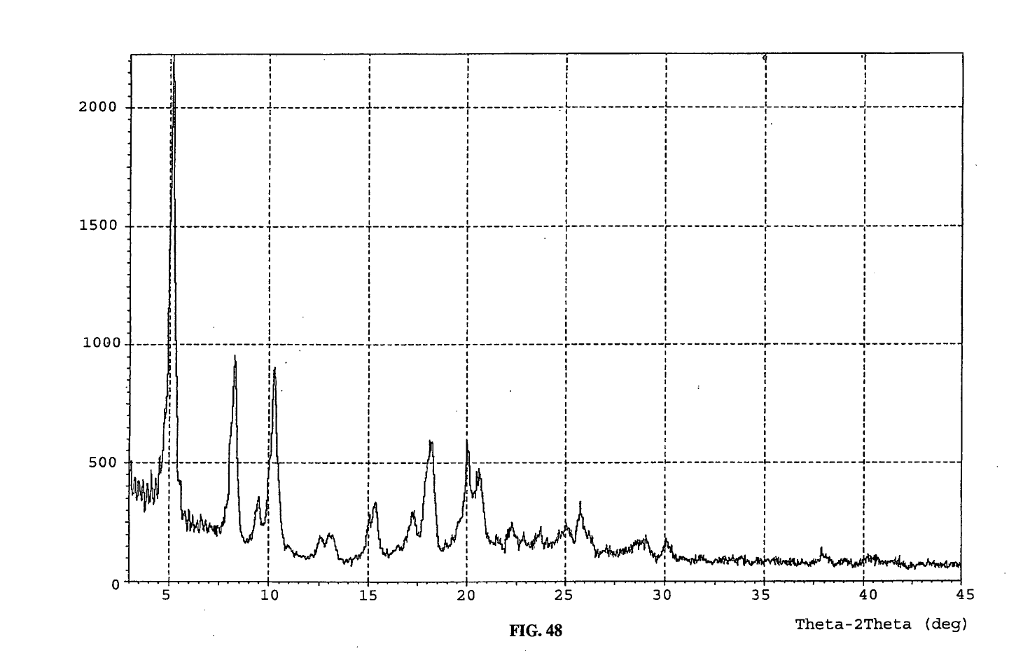

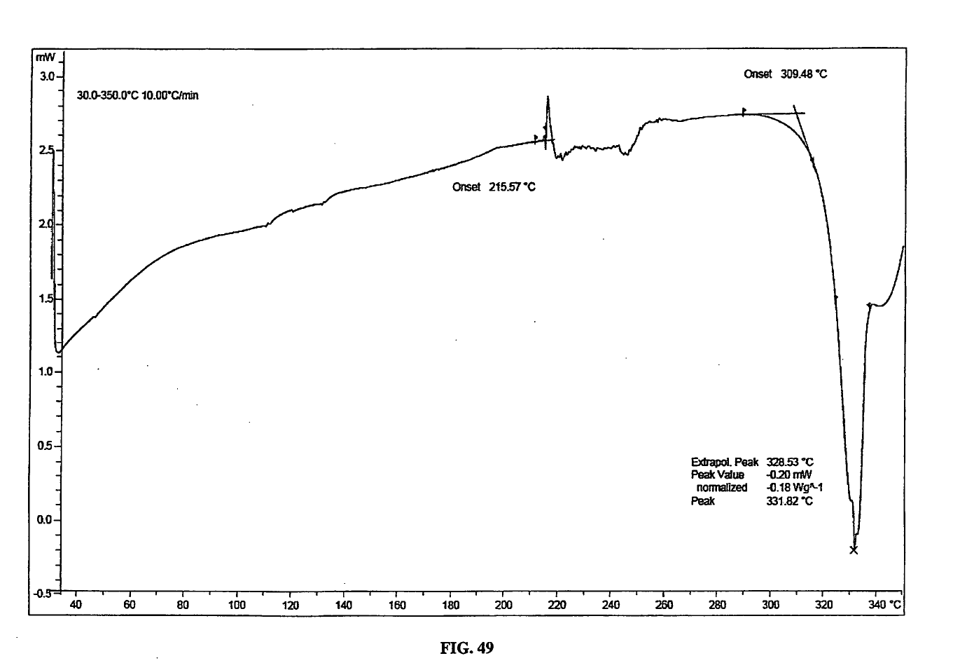

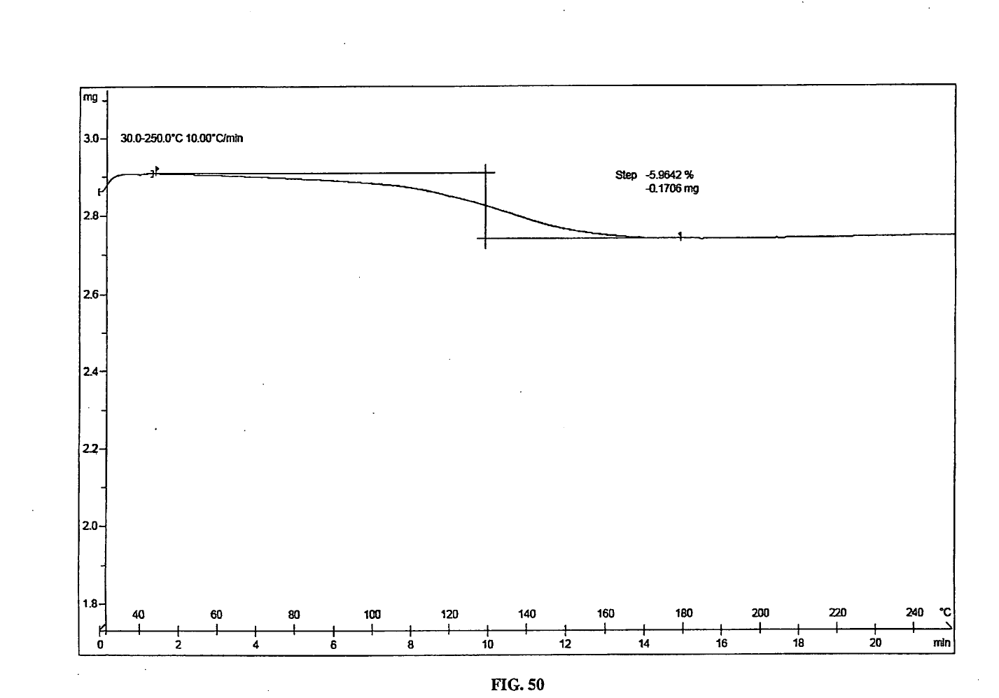

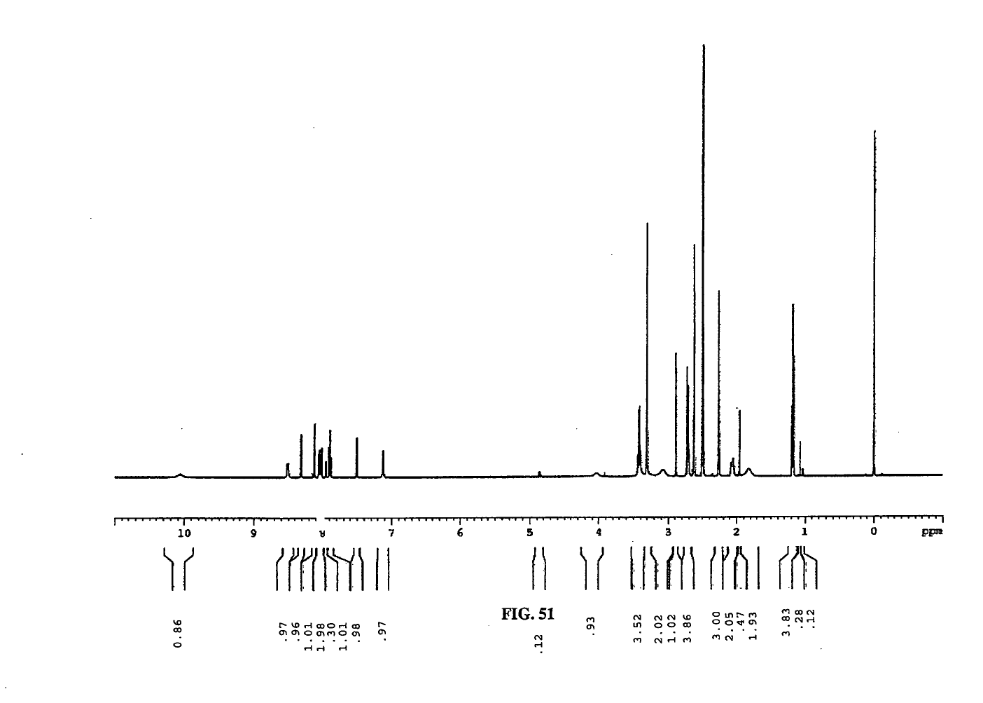

FIG. 48 shows a characteristic XRPD spectrum (CuKα, λ=1.5418 Å) of Form M. The XRPD pattern confirms that Form M is crystalline. Major X-Ray diffraction lines expressed in °2θ and their relative intensities are summarized in Table 12.

TABLE 12
Characteristic XRPD Peaks (CuKα) of Form M

| Peak No. | 2θ (°) | d-spacing | Intensity | I/Iₒ |
|---|---|---|---|---|
| 1 | 4.4800 | 19.70812 | 84 | 988 |
| 2 | 4.6400 | 19.02888 | 167 | 0 |
| 3 | 5.1309 | 17.20934 | 1349 | 13342 |
| 4 | 5.6000 | 15.76875 | 48 | 896 |
| 5 | 8.2465 | 10.71316 | 551 | 5560 |
| 6 | 9.4816 | 9.32023 | 136 | 1393 |
| 7 | 10.2400 | 8.63159 | 557 | 6743 |
| 8 | 12.6000 | 7.01968 | 69 | 638 |
| 9 | 13.1200 | 6.74261 | 80 | 1022 |
| 10 | 15.0400 | 5.88589 | 118 | 1017 |
| 11 | 15.3200 | 5.77894 | 171 | 1404 |
| 12 | 17.2435 | 5.13839 | 122 | 1623 |
| 13 | 18.1029 | 4.89635 | 358 | 4950 |
| 14 | 19.5200 | 4.54397 | 72 | 713 |
| 15 | 20.0400 | 4.42722 | 309 | 3010 |
| 16 | 20.5600 | 4.31640 | 243 | 3303 |
| 17 | 22.2434 | 3.99339 | 65 | 947 |
| 18 | 23.6532 | 3.75847 | 48 | 438 |
| 19 | 24.7200 | 3.59863 | 46 | 484 |
| 20 | 25.0400 | 3.55337 | 75 | 826 |
| 21 | 25.7674 | 3.45468 | 132 | 1296 |
| 22 | 26.2400 | 3.39352 | 54 | 545 |
| 23 | 28.5600 | 3.12291 | 42 | 615 |
| 24 | 29.0000 | 3.07652 | 46 | 497 |
| 25 | 30.0818 | 2.96830 | 51 | 618 |

This unique set of XRPD peak positions or a subset thereof can be used to identify Form M. One such subset comprises peaks at about 5.1, 8.2 and 10.2°2θ. Another subset comprises peaks at about 18.1 and 20.6°2θ.

FIG. 49 shows a characteristic DSC thermogram of Form M. An endotherm onset at about 309° C. and centered at about 332° C. (peak maximum) was observed.

FIG. 50 is a TGA thermogram of Form M, showing a weight loss of approximately 6.0% at a temperature below 200° C. The theoretical weight loss for a monohydrate is 3.2%.

Form M was further characterized by solution ¹H NMR. The spectrum is reported in FIG. 51. Chemical assignments were not performed; however, the spectra are consistent with the known chemical structure of Compound 1.

Further details related to the preparation and characterization of Form M are presented below in the Examples section.

13. Form N

Based on the available characterization data, Form N appears to be an hydrate polymorphic form of Compound 1. Form N was characterized by techniques including XRPD, DSC, TGA, and ¹H-NMR. Table 36a summarizes some of these results.

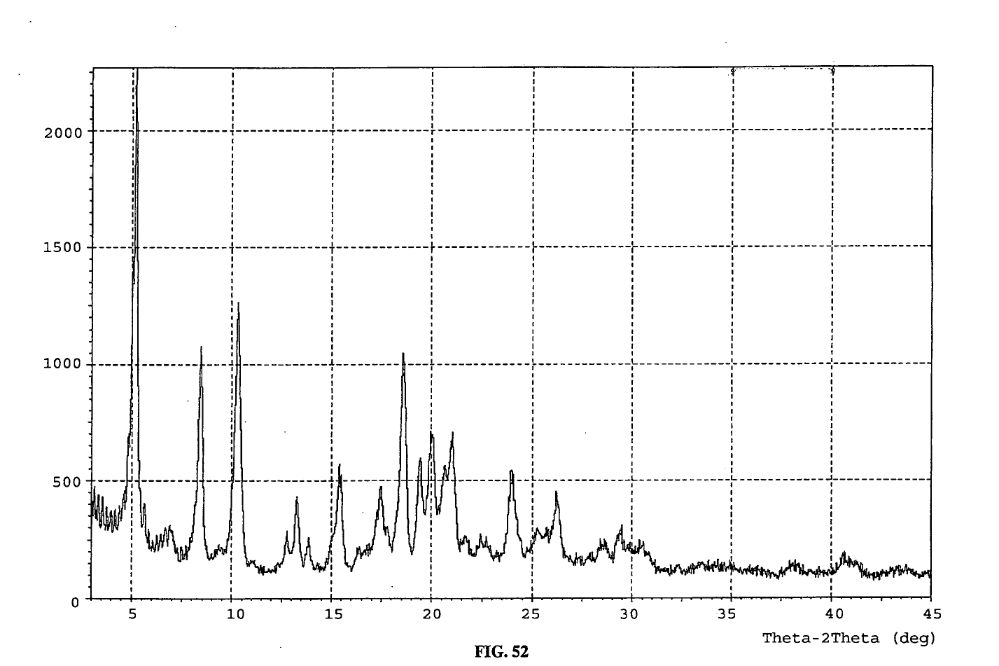

FIG. 52 shows a characteristic XRPD spectrum (CuKα, λ=1.5418 Å) of Form N. The XRPD pattern confirms that Form N is crystalline. Major X-Ray diffraction lines expressed in °2θ and their relative intensities are summarized in Table 13.

TABLE 13
Characteristic XRPD Peaks (CuKα) of Form N

| Peak No. | 2θ (°) | d-spacing | Intensity | I/Iₒ |
|---|---|---|---|---|
| 1 | 4.4800 | 19.70812 | 56 | 933 |
| 2 | 5.1562 | 17.12495 | 1350 | 11632 |
| 3 | 5.6400 | 15.65701 | 93 | 691 |
| 4 | 6.6800 | 13.22154 | 66 | 546 |
| 5 | 6.9600 | 12.69025 | 88 | 689 |
| 6 | 8.4202 | 10.49255 | 663 | 5474 |
| 7 | 9.4400 | 9.36121 | 43 | 934 |
| 8 | 10.2867 | 8.59251 | 822 | 8368 |
| 9 | 12.8000 | 6.91045 | 100 | 1018 |
| 10 | 13.2749 | 6.66428 | 221 | 1576 |
| 11 | 13.8514 | 6.38818 | 85 | 641 |
| 12 | 14.9200 | 5.93296 | 64 | 502 |
| 13 | 15.3909 | 5.75248 | 328 | 3053 |
| 14 | 16.3600 | 5.41384 | 48 | 583 |
| 15 | 17.3939 | 5.09430 | 211 | 2105 |
| 16 | 17.8000 | 4.97898 | 68 | 481 |
| 17 | 18.2000 | 4.87044 | 82 | 319 |
| 18 | 18.5708 | 4.77402 | 644 | 5287 |
| 19 | 19.4182 | 4.56756 | 279 | 2241 |
| 20 | 19.9899 | 4.43820 | 378 | 3716 |
| 21 | 20.6400 | 4.29985 | 268 | 2535 |
| 22 | 20.9600 | 4.23492 | 366 | 3328 |
| 23 | 21.6254 | 4.10610 | 55 | 605 |
| 24 | 22.4400 | 3.95885 | 58 | 399 |
| 25 | 22.7200 | 3.91069 | 45 | 344 |
| 26 | 23.9792 | 3.70810 | 284 | 2728 |
| 27 | 24.3600 | 3.65099 | 61 | 438 |
| 28 | 25.2400 | 3.52566 | 94 | 1563 |
| 29 | 25.6800 | 3.46624 | 93 | 0 |
| 30 | 26.1946 | 3.39930 | 209 | 2806 |
| 31 | 28.4000 | 3.14014 | 60 | 491 |
| 32 | 28.6000 | 3.11864 | 70 | 514 |
| 33 | 29.3610 | 3.03951 | 112 | 1487 |
| 34 | 29.8000 | 2.99573 | 67 | 0 |
| 35 | 30.4800 | 2.93043 | 79 | 1313 |
| 36 | 30.8000 | 2.90071 | 52 | 358 |
| 37 | 40.5600 | 2.22239 | 64 | 773 |
| 38 | 40.9200 | 2.20367 | 42 | 0 |
| 39 | 41.2800 | 2.18528 | 41 | 525 |

This unique set of XRPD peak positions or a subset thereof can be used to identify Form N. One such subset comprises peaks at about 5.2, 8.4 and 10.3°2θ. Another subset comprises peaks at about 18.6, 20.0 and 21.0°2θ.

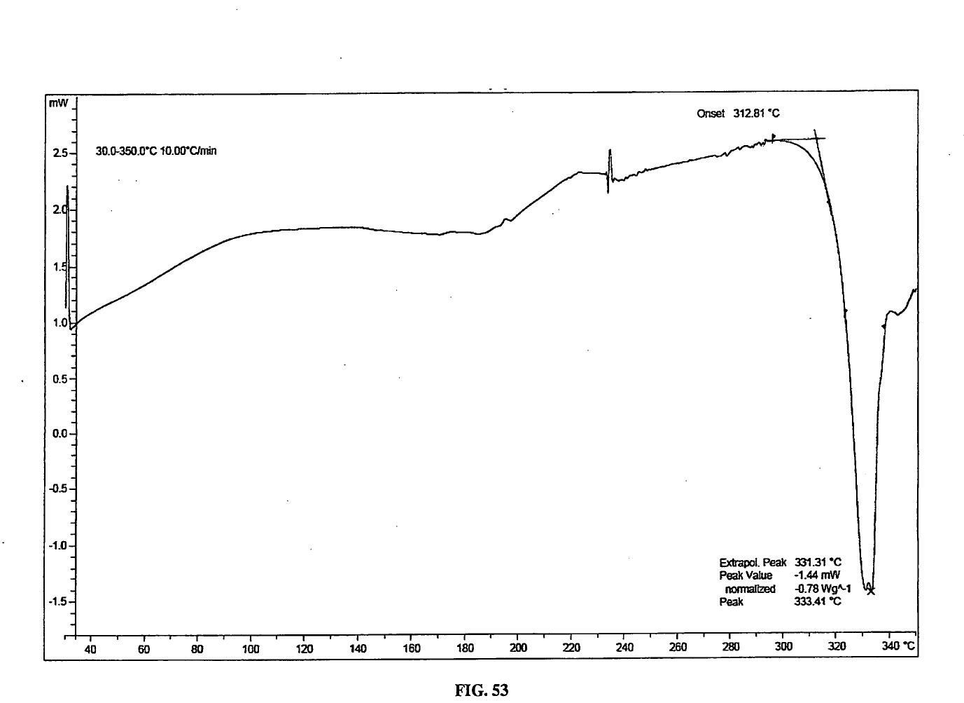

FIG. 53 shows a characteristic DSC thermogram of Form N. An endotherm which onset at about 313° C. and centered at about 333° C. (peak maximum) was observed.

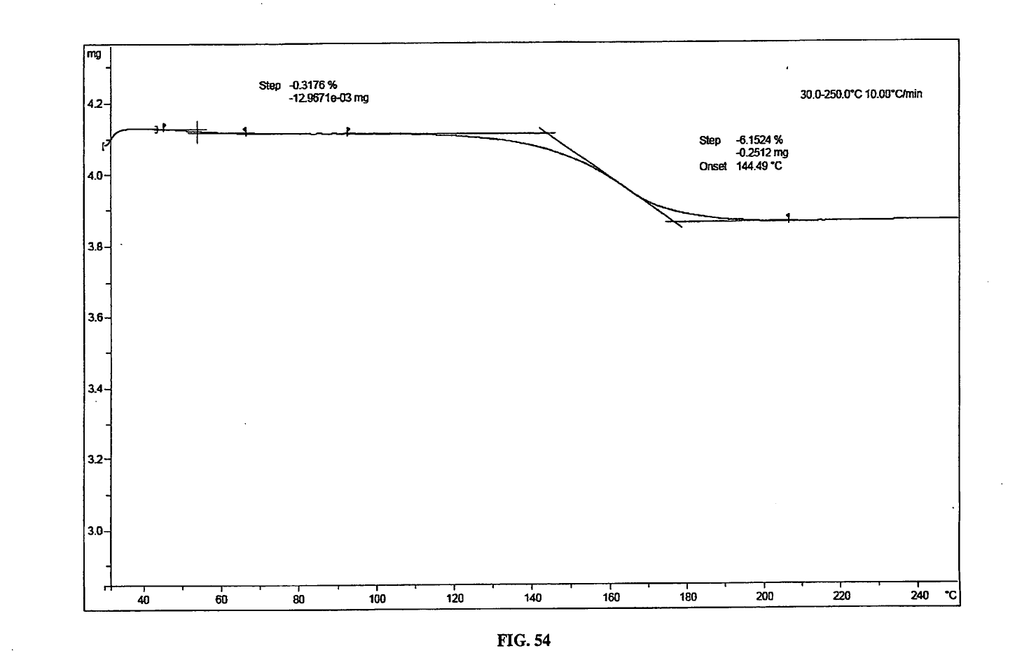

FIG. 54 is a TGA thermogram of Form N, showing a weight loss of approximately 6.2% at a temperature below 200° C. The theoretical weight loss for a monohydrate is 3.2%.

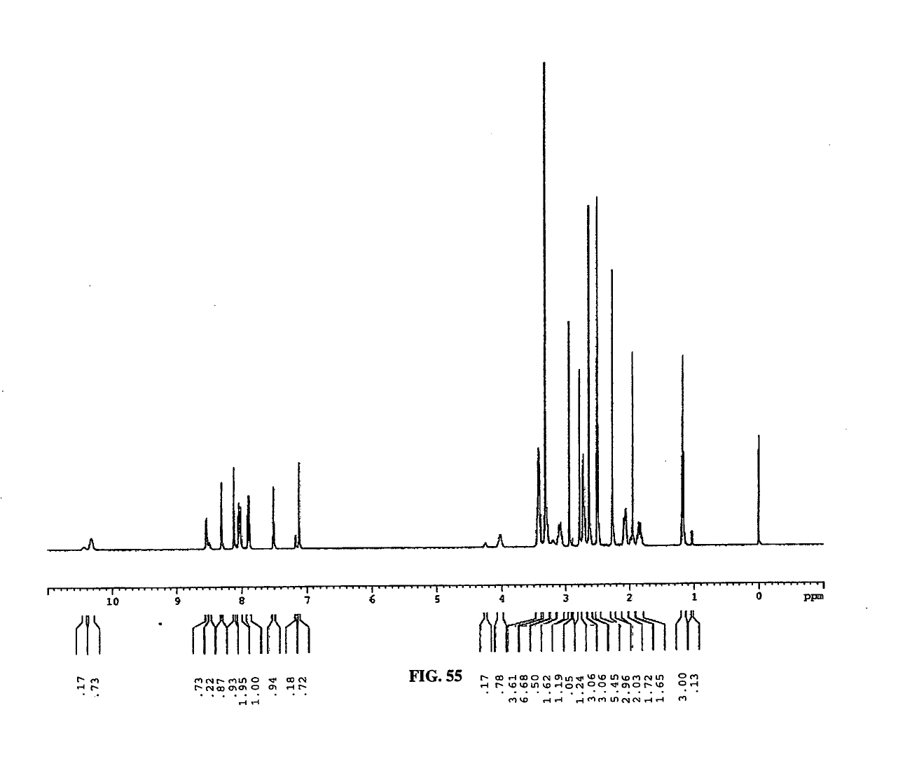

Form N was further characterized by solution ¹H NMR. The spectrum is reported in FIG. 55. Chemical assignments were not performed; however, the spectra are consistent with the known chemical structure of Compound 1.

Further details related to the preparation and characterization of Form N are presented below in the Examples section.

14. Form O

Form O appears to be a dehydrate polymorphic form of Compound 1. It can be obtained by drying Form N under the TGA conditions. Form O was characterized by techniques including XRPD, DSC and TGA. Table 36a summarizes some of these results.

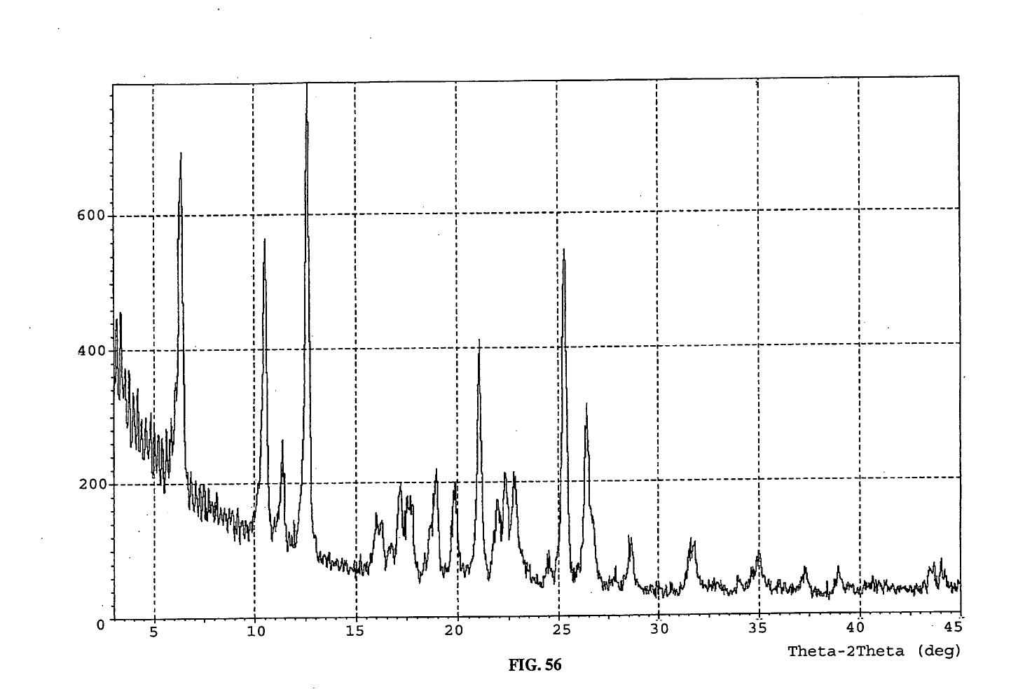

FIG. 56 shows a characteristic XRPD spectrum (CuKα, λ=1.5418 Å) of Form O. The XRPD pattern confirms that Form O is crystalline. Major X-Ray diffraction lines expressed in °2θ and their relative intensities are summarized in Table 14.

TABLE 14
Characteristic XRPD Peaks (CuKα) of Form O

| Peak No. | 2θ (°) | d-spacing | Intensity | I/Iₒ |
|---|---|---|---|---|
| 1 | 3.1200 | 28.29512 | 39 | 124 |
| 2 | 3.3622 | 26.25737 | 51 | 214 |
| 3 | 3.7593 | 23.48462 | 31 | 211 |
| 4 | 4.6121 | 19.14393 | 20 | 178 |
| 5 | 5.6613 | 15.59815 | 46 | 286 |
| 6 | 5.8400 | 15.12125 | 59 | 300 |
| 7 | 6.3387 | 13.93265 | 398 | 3172 |
| 8 | 6.8533 | 12.88759 | 22 | 71 |
| 9 | 7.4819 | 11.80618 | 20 | 124 |
| 10 | 10.1200 | 8.73367 | 49 | 307 |
| 11 | 10.5047 | 8.41467 | 352 | 2325 |
| 12 | 11.0000 | 8.03687 | 16 | 102 |
| 13 | 11.3828 | 7.76744 | 106 | 645 |
| 14 | 12.2400 | 7.22532 | 50 | 407 |
| 15 | 12.6104 | 7.01392 | 520 | 3330 |
| 16 | 13.0000 | 6.80458 | 19 | 150 |
| 17 | 15.7200 | 5.63278 | 17 | 86 |
| 18 | 16.0000 | 5.53483 | 60 | 393 |
| 19 | 16.2400 | 5.45357 | 59 | 319 |
| 20 | 16.6866 | 5.30861 | 31 | 277 |
| 21 | 17.1722 | 5.15957 | 102 | 722 |
| 22 | 17.6000 | 5.03511 | 87 | 976 |
| 23 | 18.3600 | 4.82836 | 23 | 99 |
| 24 | 18.6000 | 4.76660 | 52 | 264 |
| 25 | 18.8711 | 4.69873 | 118 | 886 |
| 26 | 19.8255 | 4.47463 | 102 | 801 |
| 27 | 21.0419 | 4.21863 | 261 | 1777 |
| 28 | 21.6800 | 4.09588 | 39 | 241 |
| 29 | 21.9200 | 4.05157 | 95 | 626 |
| 30 | 22.3353 | 3.97717 | 123 | 922 |
| 31 | 22.7755 | 3.90129 | 117 | 1174 |
| 32 | 24.4755 | 3.63403 | 23 | 142 |
| 33 | 24.8800 | 3.57585 | 28 | 106 |
| 34 | 25.2945 | 3.51819 | 407 | 2742 |
| 35 | 26.4067 | 3.37248 | 196 | 1444 |
| 36 | 26.7200 | 3.33364 | 73 | 557 |
| 37 | 28.5798 | 3.12079 | 50 | 458 |
| 38 | 31.3200 | 2.85372 | 19 | 86 |
| 39 | 31.5200 | 2.83607 | 53 | 255 |
| 40 | 31.7600 | 2.81518 | 55 | 367 |
| 41 | 33.9470 | 2.63865 | 16 | 91 |
| 42 | 34.6000 | 2.59033 | 21 | 123 |
| 43 | 34.9334 | 2.56637 | 41 | 379 |
| 44 | 37.2382 | 2.41265 | 25 | 189 |
| 45 | 38.9265 | 2.31181 | 30 | 194 |
| 46 | 43.4496 | 2.08105 | 35 | 170 |
| 47 | 43.6566 | 2.07167 | 28 | 115 |

This unique set of XRPD peak positions or a subset thereof can be used to identify Form O. One such subset comprises peaks at about 6.3, 12.6 and 25.3°2θ. Another subset comprises peaks at about 10.5 and 21.0°2θ.

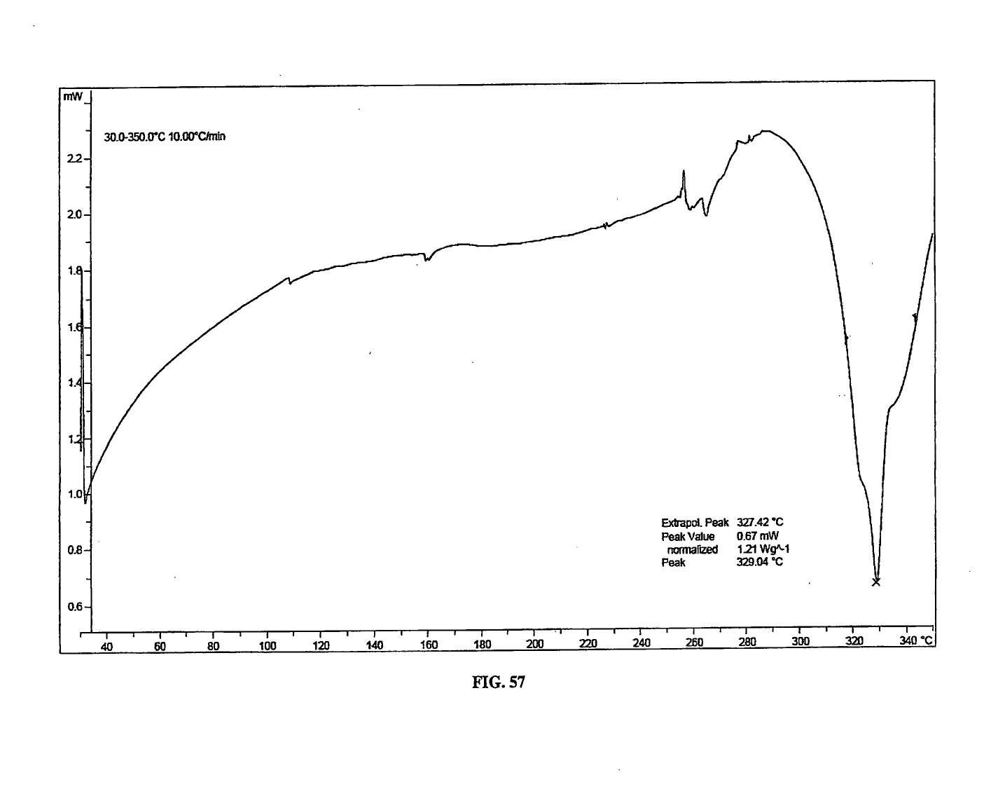

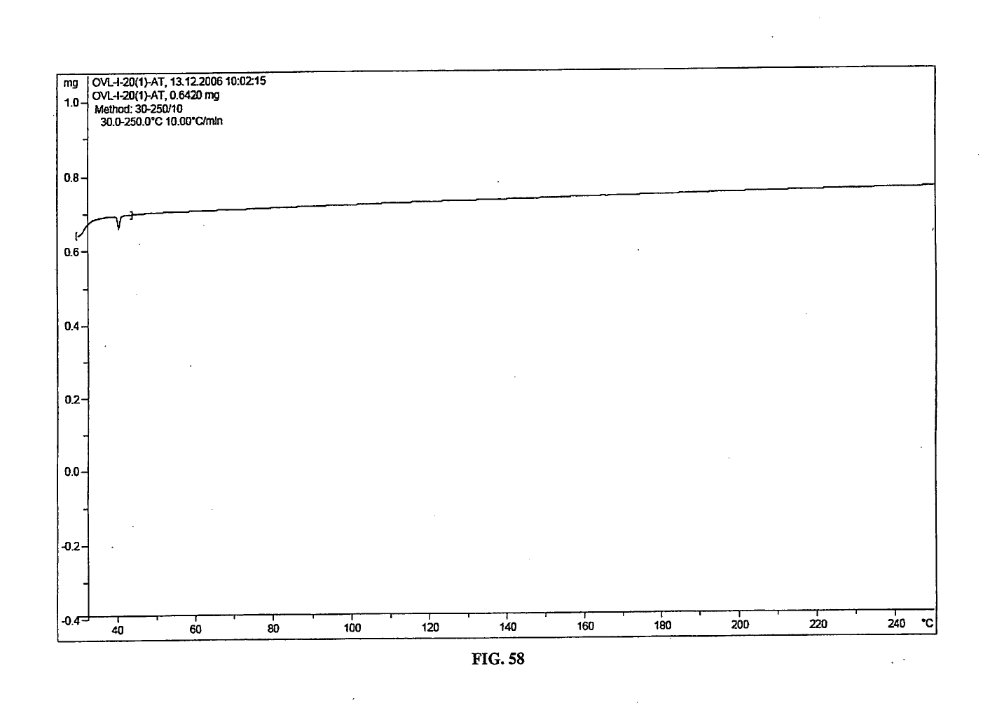

FIG. 57 shows a characteristic DSC thermogram of Form O. An endotherm was observed at approximately 327° C. (peak maximum). FIG. 58 is a TGA thermogram of Form O.

Further details related to the preparation and characterization of Form O are presented below in the Examples section.

15. Form P

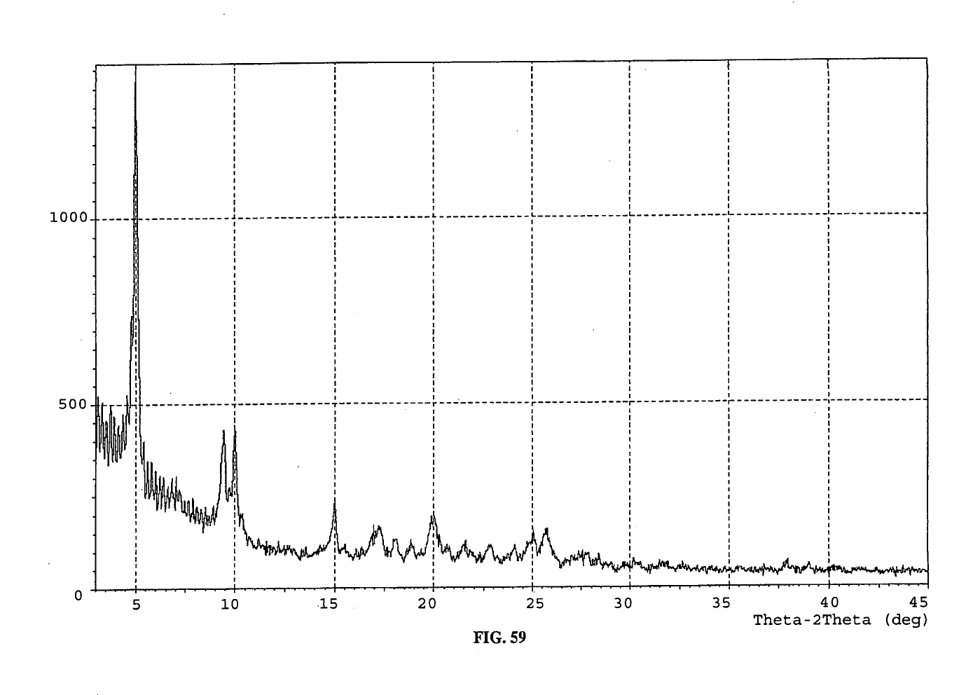

Form P appears to be a metastable form of Compound 1. Form P was characterized by techniques including XRPD. FIG. 59 shows a characteristic XRPD spectrum (CuKα, λ=1.5418 Å) of Form P. The XRPD pattern confirms that Form P is crystalline. Major X-Ray diffraction lines expressed in °2θ and their relative intensities are summarized in Table 15.

TABLE 15
Characteristic XRPD Peaks (CuKα) of Form P

| Peak No. | 2θ (°) | d-spacing | Intensity | I/Iₒ |
|---|---|---|---|---|
| 1 | 3.0991 | 28.48590 | 49 | 145 |
| 2 | 3.3181 | 26.60625 | 54 | 133 |
| 3 | 3.5309 | 25.00321 | 37 | 153 |
| 4 | 4.3600 | 20.25027 | 69 | 604 |
| 5 | 5.0151 | 17.60644 | 825 | 6787 |
| 6 | 5.6008 | 15.76650 | 36 | 311 |
| 7 | 6.4000 | 13.79934 | 29 | 159 |
| 8 | 6.8318 | 12.92810 | 35 | 240 |
| 9 | 7.2314 | 12.21458 | 35 | 190 |
| 10 | 8.5233 | 10.36586 | 26 | 160 |
| 11 | 8.8800 | 9.95026 | 32 | 168 |
| 12 | 9.4048 | 9.39616 | 203 | 1815 |
| 13 | 9.9851 | 8.85136 | 224 | 1682 |
| 14 | 10.3600 | 8.53188 | 54 | 490 |
| 15 | 14.6400 | 6.04580 | 27 | 426 |
| 16 | 14.9594 | 5.91742 | 106 | 809 |
| 17 | 16.8400 | 5.26059 | 39 | 0 |
| 18 | 17.2400 | 5.13943 | 72 | 901 |
| 19 | 18.0443 | 4.91212 | 45 | 442 |
| 20 | 18.8254 | 4.71003 | 34 | 372 |
| 21 | 19.7600 | 4.48931 | 60 | 533 |
| 22 | 20.0400 | 4.42722 | 91 | 523 |
| 23 | 20.3200 | 4.36684 | 35 | 187 |
| 24 | 20.6410 | 4.29965 | 28 | 224 |
| 25 | 21.4996 | 4.12984 | 32 | 388 |
| 26 | 22.7816 | 3.90025 | 35 | 337 |
| 27 | 24.0405 | 3.69879 | 27 | 178 |
| 28 | 24.7600 | 3.59291 | 36 | 257 |
| 29 | 25.0000 | 3.55896 | 56 | 379 |
| 30 | 25.6989 | 3.46373 | 71 | 1049 |
| 31 | 27.7606 | 3.21100 | 30 | 503 |
| 32 | 28.3785 | 3.14247 | 27 | 187 |
| 33 | 37.8316 | 2.37616 | 26 | 251 |

This unique set of XRPD peak positions or a subset thereof can be used to identify Form P. One such subset comprises peaks at about 5.0, 9.4 and 10.0°2θ. Another subset comprises peaks at about 17.2 and 25.7°2θ.

Further details related to the preparation and characterization of Form P are presented below in the Examples section.

### Indications for Use of Compound 1

The present invention also relates to methods to alter, preferably to reduce kinase activity within a subject by administrating Compound 1 in a form selected from the group consisting of Forms A, B, C, D, E, F, G, 1, J, K, L, M, N, O, and P and Amorphous Form.

Kinases are believed to contribute to the pathology and/or symptomology of several different diseases such that reduction of the activity of one or more kinases in a subject through inhibition may be used to therapeutically address these disease states. Examples of various diseases that may be treated using Compound 1 of the present invention are described herein. It is noted that additional diseases beyond those disclosed herein may be later identified as the biological roles that kinases play in various pathways becomes more fully understood.

Compound 1 may be used to treat or prevent cancer. In one embodiment, Compound 1 is used in a method comprising administering a therapeutically effective amount of Compound 1 or a composition comprising Compound 1 to a mammalian species in need thereof. In particular embodiments, the cancer is selected from the group consisting of squamous cell carcinoma, astrocytoma, Kaposi's sarcoma, glioblastoma, small-cell lung cancer, non small-cell lung cancers (e.g., large cell lung cancer, adenocarcinoma and squamous cell carcinoma), bladder cancer, head and neck cancer, melanoma, ovarian cancer, prostate cancer, breast cancer, glioma, colorectal cancer, genitourinary cancer, gastrointestinal cancer, thyroid cancer, skin cancer, kidney cancer, rectal cancer, colonic cancer, cervical cancer, mesothelioma, pancreatic cancer, liver cancer, uterus cancer, cerebral tumor cancer, urinary bladder cancer and blood cancers including multiple myeloma, chronic myelogenous leukemia and acute lymphocytic leukemia. In other embodiments, Compound 1 is useful for inhibiting growth of cancer, for suppressing metastasis of cancer, for suppressing apoptosis and the like.

In another embodiment, Compound 1 is used in a method for treating inflammation, inflammatory bowel disease, psoriasis, or transplant rejection, comprising administration to a mammalian species in need thereof a therapeutically effective amount of Compound 1 or a composition comprising Compound 1.

In another embodiment, Compound 1 is used in a method for preventing or treating amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss and contraceptive medication, comprising administration to a mammalian species in need thereof of a therapeutically effective amount of Compound 1 or a composition comprising Compound 1.

In yet another embodiment, Compound 1 is used in a method for preventing or treating mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment and androgenetic alopecia, comprising administering to a mammal, including man in need of such prevention and/or treatment, a therapeutically effective amount of Compound 1 or a composition comprising Compound 1.

In a further embodiment, Compound 1 is used in a method for preventing or treating dementia related diseases, Alzheimer's Disease and conditions associated with kinases, comprising administration to a mammalian species in need thereof of a therapeutically effective amount of Compound 1 or a composition comprising Compound 1. In one particular variation, the dementia related diseases are selected from the group consisting of Frontotemporal dementia Parkinson's Type, Parkinson dementia complex of Guam, HIV dementia, diseases with associated neurofibrillar tangle pathologies, predemented states, vascular dementia, dementia with Lewy bodies, Frontotemporal dementia and dementia pugilistica.

In another embodiment, Compound 1 is used in a method for treating arthritis comprising administration to a mammalian species in need thereof of a therapeutically effective amount of Compound 1 or a composition comprising Compound 1.

Compositions, according to the present invention, may be administered, or coadministered with other active agents. These additional active agents may include, for example, one or more other pharmaceutically active agents. Coadministration in the context of this invention is intended to mean the administration of more than one therapeutic agent, one of which includes Compound 1. Such co-administration may also be coextensive, that is, occurring during overlapping periods of time or may be sequential, that is, occurring during non-overlapping periods of time. Examples of co-administration of Compound 1 with other active ingredients in a combination therapy are described in U.S. Patent Publication No. 2007-0117816, published May 24, 2007 (see Compound 112) and U.S. Patent Application Nos. 60/912,625 and 60/912,629, filed Apr. 18, 2007 (see Compound 83), which are incorporated herein by reference in their entireties.

For oncology indications, Compound 1 may be administered in conjunction with other agents to inhibit undesirable and uncontrolled cell proliferation. Examples of other anti-cell proliferation agents that may be used in conjunction with Compound 1 include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-1, tissue inhibitor of metalloproteinase-2, plasminogen activator inhibitor-1, plasminogen activator inhibitor-2, cartilage-derived inhibitor, paclitaxel, platelet factor 4, protamine sulfate (clupeine), sulfated chitin derivatives (prepared from queen crab shells), sulfated polysaccharide peptidoglycan complex (sp-pg), staurosporine, modulators of matrix metabolism, including for example, proline analogs ((l-azetidine-2-carboxylic acid (LACA)), cishydroxyproline, d,l-3,4-dehydroproline, thiaproline, beta.-aminopropionitrile fumarate, 4-propyl-5-(4-pyridinyl)-2(3H)-oxazolone, methotrexate, mitoxantrone, heparin, interferons, 2 macroglobulin-serum, chimp-3, chymostatin, beta.-cyclodextrin tetradecasulfate, eponemycin; fumagillin, gold sodium thiomalate, d-penicillamine (CDPT), beta.-1-anticollagenase-serum, alpha.2-antiplasmin, bisantrene, lobenzarit disodium, n-2-carboxyphenyl-4-chloroanthronilic acid disodium or “CCA”, thalidomide; angostatic steroid, carboxyaminoimidazole; metalloproteinase inhibitors such as BB94. Other anti-angiogenesis agents that may be used include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

In another embodiment, a therapeutic method is provided that comprises administering Compound 1. In another embodiment, a method of inhibiting cell proliferation is provided that comprises contacting a cell with an effective amount of Compound 1. In another embodiment, a method of inhibiting cell proliferation in a patient is provided that comprises administering to the patient a therapeutically effective amount of Compound 1.

In another embodiment, a method of treating a condition in a patient which is known to be mediated by one or more kinases, or which is known to be treated by kinase inhibitors, is provided comprising administering to the patient a therapeutically effective amount of Compound 1. In another embodiment, a method is provided for using Compound 1 in order to manufacture a medicament for use in the treatment of a disease state which is known to be mediated by one or more kinases, or which is known to be treated by kinase inhibitors.

In another embodiment, a method is provided for treating a disease state for which kinases possess activity that contributes to the pathology and/or symptomology of the disease state, the method comprising: administering Compound 1 to a subject such that Compound 1 is present in the subject in a therapeutically effective amount for the disease state.

The present invention relates generally to a method comprising administering between 1 mg/day and 500 mg/day of Compound 1 to a patient, optionally between 1 mg/day and 400 mg/day of Compound 1, optionally between 1 mg/day and 250 mg/day of Compound 1, optionally between 2.5 mg/day and 200 mg/day of Compound 1, optionally between 2.5 mg/day and 150 mg/day of Compound 1, and optionally between 5 mg/day and 100 mg/day of Compound 1 (in each instance based on the molecular weight of the free base form of Compound 1). Specific dosage amounts that may be used include, but are not limited to 2.5 mg, 5 mg, 6.25 mg, 10 mg, 12.5 mg, 20 mg, 25 mg, 50 mg, 75 mg, 100 mg, 200 mg, 250 mg, 400 mg and 500 mg of Compound 1 per day. It is noted that the dosage may be administered as a daily dose or weekly dose, once daily or multiple doses per day. It is noted that Compound 1 may be administered in a form selected from the group consisting of Forms A, B, C, D, E, F, G, I, J, K, L, M, N, O, and P and Amorphous Form. However, the dosage amounts and ranges provided herein are always based on the molecular weight of the free base form of Compound 1.

Compound 1 may be administered by any route of administration. In particular embodiments, however, the method of the present invention is practiced by administering Compound 1 orally.

Pharmaceutical Compositions Comprising Compound 1 where at Least One of Form A Through Form P, or Amorphous Form is Present

Compound 1 may be used in various pharmaceutical compositions where at least a portion of Compound 1 is present in the composition in a form selected from the group consisting of Forms A, B, C, D, E, F, G, I, J, K, L, M, N, O, and P and Amorphous Form. The pharmaceutical composition should contain a sufficient quantity of Compound 1 to reduce kinase activity in vivo sufficiently to provide the desired therapeutic effect. Such pharmaceutical compositions may comprise Compound 1 present in the composition in a range of between 0.005% and 100% (weight/weight), optionally 0.1-95%, and optionally 1-95%.

In particular embodiments, the pharmaceutical compositions comprise at least 0.1%, 0.25%, 0.5%, 1%, 5%, 10%, 25%, 50%, 75%, 80%, 85%, 90%, 95%, 97%, or 99% of Compound 1 in a form selected from the group consisting of Form A, Form B, Form C, Form D, Form E, Form F, Form G, From I, Form J, Form K, Form L, Form M, Form N, Form O, Form P, Amorphous Form, and mixtures thereof. In another embodiment, a particular polymorphic form selected from the group consisting of Form A, Form B, Form C, Form D, Form E, Form F, Form G, From I, Form J, Form K, Form L, Form M, Form N, Form O, Form P, Amorphous Form, and mixtures thereof may comprise at least 0.1%, 0.25%, 0.5%, 1%, 5%, 10%, 25%, 50%, 75%, 80%, 85%, 90%, 95%, 97%, or 99% of the total amount of Compound 1 (weight/weight) in the pharmaceutical composition.

In addition to Compound 1, the pharmaceutical composition may comprise one or more additional components that do not deleteriously affect the use of Compound 1. For example, the pharmaceutical compositions may include, in addition to Compound 1, conventional pharmaceutical carriers; excipients; diluents; lubricants; binders; wetting agents; disintegrating agents; glidants; sweetening agents; flavoring agents; emulsifying agents; solubilizing agents; pH buffering agents; perfuming agents; surface stabilizing agents; suspending agents; and other conventional, pharmaceutically inactive agents. In particular, the pharmaceutical compositions may comprise lactose, mannitol, glucose, sucrose, dicalcium phosphate, magnesium carbonate, sodium saccharin, carboxymethylcellulose, magnesium stearate, calcium stearate, sodium crosscarmellose, talc, starch, natural gums (e.g., gum acaciagelatin), molasses, polyvinylpyrrolidine, celluloses and derivatives thereof, povidone, crospovidones acetate, sodium citrate, cyclodextrine derivatives, sorbitan monolaurate, triethanolamine sodium acetate, triethanolamine oleate, biocompatible polymers, such as collagen, ethylene vinyl acetate, polyanhydrides, polyglycolic acid, polyorthoesters, polylactic acid and others such agents.

Pharmaceutical compositions according to the present invention may be adapted for administration by any of a variety of routes. For example, pharmaceutical compositions according to the present invention can be administered orally, parenterally, intraperitoneally, intravenously, intraarterially, topically, transdermally, sublingually, intramuscularly, rectally, transbuccally, intranasally, liposomally, via inhalation, vaginally, intraoccularly, via local delivery (for example, by catheter or stent), subcutaneously, intraadiposally, intraarticularly, or intrathecally, optionally in a slow release dosage form. In particular embodiments, the pharmaceutical compounds are administered orally, by inhalation or by injection subcutaneously, intramuscularly, intravenously or directly into the cerebrospinal fluid.

In general, the pharmaceutical compositions of the present invention may be prepared in a gaseous, liquid, semi-liquid, gel, or solid form, and formulated in a manner suitable for the route of administration to be used.

Compositions according to the present invention are optionally provided for administration to humans and animals in unit dosage forms or multiple dosage forms, such as tablets, capsules, pills, powders, dry powders for inhalers, granules, sterile parenteral solutions or suspensions, oral solutions or suspensions, oil-water emulsions, sustained release formulations, such as, but not limited to, implants and microencapsulated delivery systems, containing suitable quantities of Compound 1. Methods of preparing such dosage forms are known in the art, and will be apparent to those skilled in this art; for example, see Remington's Pharmaceutical Sciences, 19th Ed. (Easton, Pa.: Mack Publishing Company, 1995).

Unit-dose forms, as used herein, refers to physically discrete units suitable for human and animal subjects and packaged individually as is known in the art. Each unit-dose contains a predetermined quantity of Compound 1 sufficient to produce the desired therapeutic effect, in association with a pharmaceutical carrier, vehicle or diluent. Examples of unit-dose forms include ampoules and syringes, and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules, or bottles of pints or gallons. Hence, multiple dose form may be viewed as a multiple of unit-doses that are not segregated in packaging.

In general, the total amount of Compound 1 in a pharmaceutical composition according to the present invention should be sufficient to provide a desired therapeutic effect. This amount may be delivered as a single per day dosage, multiple dosages per day to be administered at intervals of time, or as a continuous release dosage form. Compound 1 may advantageously be used when administered to a patient at a daily dose of between 1 mg/day and 250 mg/day of Compound 1, optionally between 2.5 mg and 200 mg of Compound 1, optionally between 2.5 mg and 150 mg of Compound 1, and optionally between 5 mg and 100 mg of Compound 1 (in each instance based on the molecular weight of the free base form of Compound 1). Specific dosage amounts that may be used include, but are not limited to 2.5 mg, 5 mg, 6.25 mg, 10 mg, 12.5 mg, 20 mg, 25 mg, 50 mg, 75 mg, and 100 mg of Compound 1 per day. It may be desirable for Compound 1 to be administered one time per day. Accordingly, pharmaceutical compositions of the present invention may be in the form of a single dose form comprising between 1 mg/day and 250 mg/day of Compound 1, optionally between 2.5 mg and 200 mg of Compound 1, optionally between 2.5 mg and 150 mg of Compound 1, and optionally between 5 mg and 100 mg of Compound 1. In specific embodiments, the pharmaceutical composition comprises 2.5 mg, 5 mg, 6.25 mg, 10 mg, 12.5 mg, 20 mg, 25 mg, 50 mg, 75 mg or 100 mg of Compound 1.

A. Formulations for Oral Administration

Oral pharmaceutical dosage forms may be as a solid, gel or liquid where at least a portion of Compound 1 is present in the composition in a form selected from the group consisting of Form A, Form B, Form C, Form D, Form E, Form F, Form G, From I, Form J, Form K, Form L, Form M, Form N, Form O, Form P, and Amorphous Form.

In certain embodiments, Compound 1 is provided as solid dosage forms. Examples of solid dosage forms include, but are not limited to pills, tablets, troches, capsules, granules, and bulk powders. More specific examples of oral tablets include compressed, chewable lozenges, troches and tablets that may be enteric-coated, sugar-coated or film-coated. Examples of capsules include hard or soft gelatin capsules. Granules and powders may be provided in non-effervescent or effervescent forms. The powders may be prepared by lyophilization or by other suitable methods.

The tablets, pills, capsules, troches and the like may optionally contain one or more of the following ingredients, or compounds of a similar nature: a binder; a diluent; a disintegrating agent; a lubricant; a glidant; a coloring agent; a sweetening agent; a flavoring agent; and a wetting agent.

Examples of binders that may be used include, but are not limited to, microcrystalline cellulose, gum tragacanth, glucose solution, acacia mucilage, gelatin solution, sucrose and starch paste.

Examples of diluents that may be used include, but are not limited to, lactose, sucrose, starch, kaolin, salt, mannitol and dicalcium phosphate.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of lubricants that may be used include, but are not limited to, talc, starch, magnesium or calcium stearate, lycopodium and stearic acid.

Examples of glidants that may be used include, but are not limited to, colloidal silicon dioxide.

Examples of coloring agents that may be used include, but are not limited to, any of the approved certified water soluble FD and C dyes, mixtures thereof; and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

When the dosage form is a pill, tablet, torches, or the like, Compound 1 may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compound 1 may also be administered as a component of an elixir, emulsion, suspension, microsuspension, syrup, wafer, sprinkle, chewing gum or the like. A syrup may optionally comprise, in addition to the active compounds, sucrose as a sweetening agent and certain preservatives, dyes and colorings and flavors.

Alternatively, liquid or semi-solid oral formulations may be prepared by dissolving or dispersing the active compound or salt in vegetable oils, glycols, triglycerides, propylene glycol esters (e.g. propylene carbonate) and other such carriers, and encapsulating these solutions or suspensions in hard or soft gelatin capsule shells. Other useful formulations include those set forth in U.S. Pat. Nos. Re 28,819 and 4,358,603.

Examples of oral formulations that may be used to administer Compound 1 has been described in U.S. patent application Ser. No. 11/531,671, filed Sep. 13, 2006, the disclosure of which is herein expressly incorporated by reference in its entirety.

Exemplary tablet formulations are provided below. It is noted that the examples are, by way of illustration but not limitation. It is also noted that Compound 1 is present in the formulation in a form selected from the group consisting of one or more of Form A, Form B, Form C, Form D, Form E, Form F, Form G, From I, Form J, Form K, Form L, Form M, Form N, Form O, Form P, and Amorphous Form. It is also noted that the formulations provided herein may be varied as is known in the art.

12.5 mg of Compound 1 (weight of free base form) per tablet
Core Tablet Formulation

| | | |
|---|---|---|
| (1) | Compound 1 | 17.0 mg |
| (2) | Lactose Monohydrate, NF, Ph, Eur | 224.6 mg |
| | (FOREMOST 316 FAST FLO) | |
| (3) | Microcrystalline Cellulose, NF, Ph, Eur | 120.1 mg |
| | (AVICEL PH 102) | |
| (4) | Croscarmellose Sodium, NF, Ph, Eur | 32.0 mg |
| | (AC-DO-SOL) | |
| (5) | Colloidal Silicon Dioxide, NF, Ph, Eur | 3.2 mg |
| | (CAB-O-SIL M-5P) | |
| (6) | Magnesium Stearate, NF, Ph, Eur | 3.2 mg |
| | (MALLINCKRODT, Non-bovine Hyqual) | |

| | |
|---|---|
| TOTAL (per tablet) | 400.0 mg |

25 mg of Compound 1 (weight of free base form) per tablet
Core Tablet Formulation

| | | |
|---|---|---|
| (1) | Compound 1 | 34.0 mg |
| (2) | Lactose Monohydrate, NF, Ph, Eur | 207.6 mg |
| | (FOREMOST 316 FAST FLO) | |
| (3) | Microcrystalline Cellulose, NF, Ph, Eur | 120.1 mg |
| | (AVICEL PH 102) | |
| (4) | Croscarmellose Sodium, NF, Ph, Eur | 32.0 mg |
| | (AC-DO-SOL) | |
| (5) | Colloidal Silicon Dioxide, NF, Ph, Eur | 3.2 mg |
| | (CAB-O-SIL M-5P) | |
| (6) | Magnesium Stearate, NF, Ph, Eur | 3.2 mg |
| | (MALLINCKRODT, Non-bovine Hyqual) | |

| | |
|---|---|
| TOTAL (per tablet) | 400.0 mg |

50 mg of Compound 1 (weight of free base form) per tablet
Core Tablet Formulation

| | | |
|---|---|---|
| (1) | Compound 1 | 68.0 mg |
| (2) | Lactose Monohydrate, NF, Ph, Eur | 173.6 mg |
| | (FOREMOST 316 FAST FLO) | |
| (3) | Microcrystalline Cellulose, NF, Ph, Eur | 120.1 mg |
| | (AVICEL PH 102) | |
| (4) | Croscarmellose Sodium, NF, Ph, Eur | 32.0 mg |
| | (AC-DO-SOL) | |
| (5) | Colloidal Silicon Dioxide, NF, Ph, Eur | 3.2 mg |
| | (CAB-O-SIL M-5P) | |
| (6) | Magnesium Stearate, NF, Ph, Eur | 3.2 mg |
| | (MALLINCKRODT, Non-bovine Hyqual) | |

| | |
|---|---|
| TOTAL (per tablet) | 400.0 mg |

Film Coat (12.0 mg in total)
(1) Opadry II 85F18422, White - Portion 1 (COLORCON)
(2) Opadry II 85F18422, White - Portion 2 (COLORCON)
(3) Opadry II 85F18422, White - Portion 3 (COLORCON)

B. Injectables, Solutions and Emulsions

Compound 1 present in a form or a mixture of forms selected from the group consisting of Form A, Form B, Form C, Form D, Form E, Form F, Form G, From I, Form J, Form K, Form L, Form M, Form N, Form O, Form P, and Amorphous Form may be formulated for parenteral administration. Parenteral administration generally characterized by injection, either subcutaneously, intramuscularly or intravenously. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the route of administration and the indication of disease to be treated.

Injectables may be prepared in any conventional form. These formulations include, but are not limited to, sterile solutions, suspensions, microsuspensions, and emulsions ready for injection, and solid forms, e.g., lyophilized or other powders including hypodermic tablets, ready to be combined with a carrier just prior to use. Generally, the resulting formulation may be a solution, microsuspension, suspension and emulsion. The carrier may be an aqueous, non-aqueous liquid, or a solid vehicle that can be suspended in liquid.

Examples of carriers that may be used in conjunction with injectables according to the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions includes EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of Compound 1 in the parenteral formulation may be adjusted so that an injection administers a pharmaceutically effective amount sufficient to produce the desired pharmacological effect. The exact concentration of Compound 1 and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is known and practiced in the art.

Injectables may be designed for local and systemic administration. Typically a therapeutically effective dosage is formulated to contain a concentration of at least about 0.1% w/w up to about 90% w/w or more, preferably more than 1% w/w of Compound 1 to the treated tissue(s). Compound 1 may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

Compound 1 may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

C. Powders

Compound 1 in a form or a mixture of forms selected from the group consisting of one or more of Form A, Form B, Form C, Form D, Form E, Form F, Form G, From I, Form J, Form K, Form L, Form M, Form N, Form O, Form P, and Amorphous Form may be prepared as powders, which can be reconstituted for administration as solutions, emulsions and other mixtures. The powders may also be formulated as solids or gels.

Powders of Compound 1 may be prepared by grinding, spray drying, lyophilization and other techniques that are well known in the art. Sterile, lyophilized powder may be prepared by dissolving Compound 1 in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, Compound 1 is added to the resulting mixture, preferably above room temperature, more preferably at about 30-35° C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of Compound 1.

D. Topical Administration

Compound 1 present in a form or a mixture of forms selected from the group consisting of Form A, Form B, Form C, Form D, Form E, Form F, Form G, From I, Form J, Form K, Form L, Form M, Form N, Form O, Form P, and Amorphous Form may also be administered as topical mixtures. Topical mixtures may be used for local and systemic administration. The resulting mixture may be a solution, suspension, microsuspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

Compound 1 may be formulated for topical applications to the respiratory tract. These pulmonary formulations can be in the form of an aerosol, solution, emulsion, suspension, microsuspension for a nebulizer, or as a microfine powder for insufflation, alone or in combination with an inert carrier such as lactose. In such a case, the particles of the formulation will typically have diameters of less than 50 microns, preferably less than 10 microns. Examples of aerosols for topical application, such as by inhalation are disclosed in U.S. Pat. Nos. 4,044,126, 4,414,209, and 4,364,923, which describe aerosols for delivery of a steroid useful for treatment inflammatory diseases, particularly asthma.

Compound 1 may also be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions or suspensions of Compound 1 alone or in combination with other pharmaceutically acceptable excipients can also be administered.

E. Formulations for Other Routes of Administration

Depending upon the disease state being treated, Compound 1 present in a form or a mixture of forms selected from the group consisting of Form A, Form B, Form C, Form D, Form E, Form F, Form G, From I, Form J, Form K, Form L, Form M, Form N, Form O, Form P, and Amorphous Form may be formulated for other routes of administration, such as topical application, transdermal patches, and rectal administration. For example, pharmaceutical dosage forms for rectal administration are rectal suppositories, capsules and tablets for systemic effect. Rectal suppositories as used herein mean solid bodies for insertion into the rectum that melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration may be manufactured using the same pharmaceutically acceptable substance and by the same methods as for formulations for oral administration.

### Kits and Articles of Manufacture Comprising Compound 1 Polymorphs

The present invention is also directed to kits and other articles of manufacture for treating diseases associated with kinases. It is noted that diseases are intended to cover all conditions for which kinases possess activity that contributes to the pathology and/or symptomology of the condition.

In one embodiment, a kit is provided that comprises a pharmaceutical composition comprising Compound 1 where greater than 0.1%, 0.25%, 0.5%, 1%, 5%, 10%, 25%, 50%, 75%, 80%, 85%, 90%, 95%, 97% or 99% of Compound 1 (by weight) is present in a form selected from the group consisting of Form A, Form B, Form C, Form D, Form E, Form F, Form G, From I, Form J, Form K, Form L, Form M, Form N, Form O, Form P, and Amorphous Form; and instructions for use of the kit. Optionally, the composition comprises at least 0.1%, 0.25%, 0.5%, 1%, 5%, 10%, 25%, 50%, 75%, 80%, 85%, 90%, 95%, 97%, or 99% of Compound 1. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a pharmaceutical composition comprising Compound 1 where greater than 0.1%, 0.25%, 0.5%, 1%, 5%, 10%, 25%, 50%, 75%, 80%, 85%, 90%, 95%, 97% or 99% of Compound 1 (by weight) is present in the composition in a form selected from the group consisting of Form A, Form B, Form C, Form D, Form E, Form F, Form G, From I, Form J, Form K, Form L, Form M, Form N, Form O, Form P, and Amorphous Form; and packaging materials. Optionally, the composition comprises at least 0.1%, 0.25%, 0.5%, 1%, 5%, 10%, 25%, 50%, 75%, 80%, 85%, 90%, 95%, 97%, or 99% of Compound 1. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXAMPLES

### Example 1

### Preparation of 5-(3-(ethylsulfonyl)phenyl)-3,8-dimethyl-n-(1-methylpiperidin-4-yl)-9h-pyrido[2,3-b]indole-7-carboxamide (Compound 1)

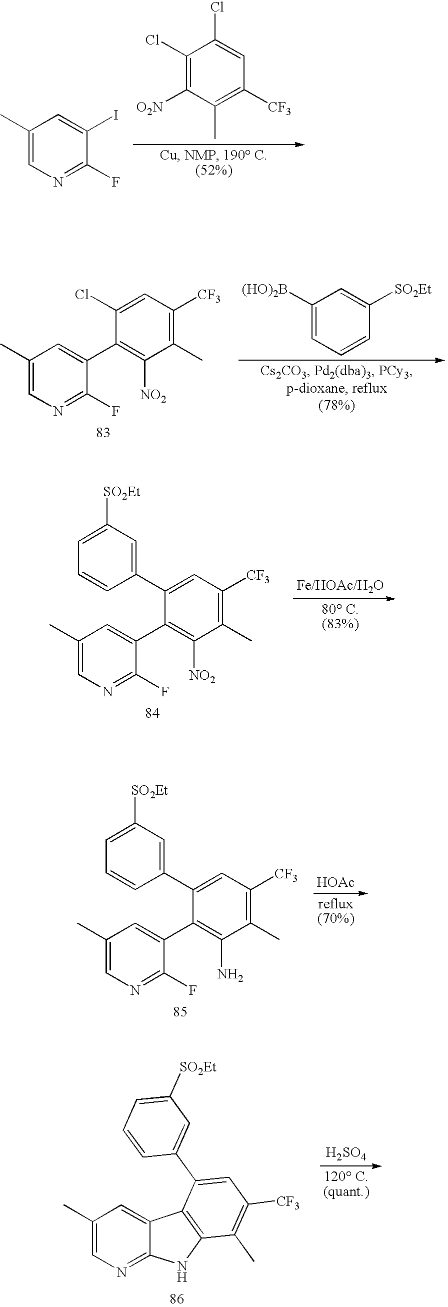

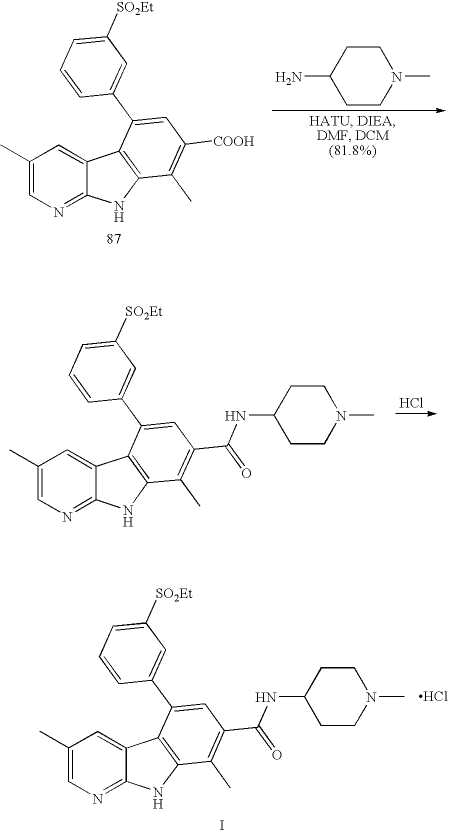

3-(6-chloro-3-methyl-2-nitro-4-(trifluoromethyl)phenyl)-2-fluoro-5-methylpyridine: 2-Fluoro-3-iodo-5-picoline (15.0 g, 63 mmol) was added drop wise during 2 h as a solution in NMP (20 mL) to a stirred suspension of 3,4-dichloro-2-nitro-6-(trifluoromethyl)-toluene (52.1 g, 190 mmol) and copper (12.1 g, 190 mmol) in NMP (115 mL) at 190° C. After completion of the reaction (2.5 h), the mixture was cooled to room temperature, filtered, rinsed with NMP (3×5 mL) followed by EtOAc (1×100 mL). The filtrate was diluted with EtOAc (400 mL) affording a turbid solution. The organic layer was partitioned with sat. NaHCO₃ (150 mL) affording a suspension/emulsion. H₂O (50 mL) and MeOH (50 mL) were added to aid solubility. The aqueous layer was washed with EtOAc (5×150 mL). The organic layers were combined, dried (MgSO₄), and concentrated in vacuo. The crude product was purified by silica gel chromatography (98:2 Toluene:EtOAc) to provide the title compound as a tan solid (11.4 g, 52%). ¹H NMR (400 MHz, DMSO-d₆): δ 8.34 (s, 1H), 8.26 (s, 1H), 7.86-7.89 (m, 1H), 2.4 (s, 3H), 2.34 (s, 3H). MS (ES) [m+H] calc'd for C₁₄H₉ClF₄N₂O₂, 349; found 349.2.

3-(3′-(ethylsulfonyl)-4-methyl-3-nitro-5-(trifluoromethyl)biphenyl-2-yl)-2-fluoro-5-methylpyridine: A mixture of Compound 83 (6.0 g, 17.2 mmol), 3-ethylsulfonylphenylboronic acid (4.79 g, 22.4 mmol), bis(dibenzylideneacetone)Pd(0) (1.48 g, 2.6 mmol), tricyclohexylphosphine (1.45 g, 5.2 mmol), Cs₂CO₃ (14.0 g, 43 mmol), and dioxane (60 mL) was heated at reflux for 4.5 hr. After completion the reaction was cooled to room temperature, filtered, rinsed with dioxane, and concentrated in vacuo. The resulting oil was reconstituted in EtOAc (75 mL) washed with H₂O (1×30 mL) and brine (1×30 mL), dried (MgSO₄), and concentrated in vacuo. The crude product was purified by silica gel chromatography (4:1 hexanes/EtOAc) to provide the title compound as a tan solid (6.5 g, 78%). ¹H NMR (400 MHz, DMSO-d₆): δ 8.15 (s, 1H), 8.04 (s, 1H), 7.90-7.93 (m, 1H), 7.80-7.82 (m, 1H), 7.60-7.70 (m, 3H), 3.1-3.2 (m, 2H), 2.49 (s, 3H), 2.25 (s, 3H), 0.85 (t, 3H). MS (ES) [m+H] calc'd for C₂₂H₁₈F₄N₂O₄S, 483; found 483.3.

3′-(ethylsulfonyl)-2-(2-fluoro-5-methylpyridin-3-yl)-4-methyl-5-(trifluoromethyl)biphenyl-3-amine: A mixture of Compound 84 (6.4 g, 13.3 mmol), iron (3.7 g, 66.3 mmol), HOAc, (32 mL), and H₂O (11 mL) was heated at 80° C. for 2 h. After completion the reaction was concentrated in vacuo. The residue was reconstituted in dichloromethane (100 mL), filtered, and rinsed with dichloromethane (3×30 mL). The organic phase was washed with sat. NaHCO₃ (1×100 mL) and brine (1×50 mL), dried (MgSO₄), filtered, and concentrated in vacuo. The crude product was purified by silica gel chromatography (1:1 hexanes/EtOAc) to provide the title compound as a tan solid (5.0 g, 83%). ¹H NMR (400 MHz, DMSO-d₆): δ 7.93 (s, 1H), 7.67-7.7.71 (m, 2H), 7.53 (t, 1H), 7.46-7.48 (m, 1H), 7.42 (s, 1H), 6.93 (s, 1H), 5.09 (s, 2H), 3.11 (q, 2H), 2.27 (s, 3H), 2.21 (s, 3H), 0.85 (t, 3H). MS (ES) [m+H] calc'd for C₂₂H₂₀F₄N₂O₂S, 453; found 453.3.

5-(3-(ethylsulfonyl)phenyl)-3,8-dimethyl-7-(trifluoromethyl)-9H-pyrido[2,3-b]indole acetate: Compound 85 (4.9 g, 10.8 mmol) was dissolved in HOAc (35 mL) and heated at reflux for 3 h. The reaction mixture was cooled to room temperature affording a crystalline product. The resulting suspension was filtered, rinsed with HOAc (3×5 mL) followed by H₂O (3×10 mL) and the solids dried in vacuo to provide the title compound as a white solid (3.73 g, 70%). NMR analysis confirmed that the product was isolated as the mono-acetate salt. ¹H NMR (400 MHz, DMSO-d₆): δ 12.35 (s, 1H), 12.0 (s, 1H), 8.39 (s, 1H), 8.15 (s, 1H), 8.04-8.09 (m, 2H), 7.90 (t, 1H), 7.51 (s, 1H), 7.42 (s, 1H), 3.43 (q, 2H), 2.76 (s, 3H), 2.28 (s, 3H), 1.91 (s, 3H), 1.18 (t, 3H). MS (ES) [m+H] calc'd for C₂₂H₁₉F₃N₂O₂S, 433; found 433.3.

5-(3-(ethylsulfonyl)phenyl)-3,8-dimethyl-9H-pyrido[2,3-b]indole-7-carboxylic acid: Compound 86 (3.6 g, 7.3 mmol) was dissolved in concentrated H₂SO₄ (30 mL) and heated at 120° C. for 30 min. The reaction was cooled to room temperature and poured over ice affording a white precipitate. The resulting suspension was filtered, rinsed with H₂O (3×30 mL) followed by IPA (3×10 mL) and dried in vacuo to provide the title compound as a white solid (3.2 g, quant.). ¹H NMR (400 MHz, DMSO-d₆): δ 12.20 (s, 1H), 8.36 (s, 1H), 8.12 (s, 1H), 8.02-8.07 (m, 2H), 7.89 (t, 1H), 7.61 (s, 1H), 7.54 (s, 1H), 3.43 (q, 2H), 2.85 (s, 3H), 2.28 (s, 3H), 1.18 (t, 3H). MS (ES) [m+H] calc'd for C₂₂H₂₀N₂O₄S, 409; found 409.3.

5-(3-(ethylsulfonyl)phenyl)-3,8-dimethyl-N-(1-methylpiperidin-4-yl)-9H-pyrido[2,3-b]indole-7-carboxamide: A mixture of Compound 87 (11.3 g, 27.6 mmol), 1-methylpiperidin-4-amine (9.47 g, 82.9 mmol), HATU (13.66 g, 35.9 mmol), DIEA (17.88 g, 138 mmol), DMF (250 mL), and DCM (250 mL) was stirred at room temperature for 30 minutes. The resulting suspension was filtered, rinsed with DMF (10 mL×4) and concentrated in vacuo. The residue was dissolved in DMSO (77 mL), filtered, and the filtrate was purified by preparative HPLC (ACN/H₂O with TFA). Following HPLC purification, the pure fractions were combined, basified with sodium bicarbonate and concentrated in vacuo to half volume. The resulting suspension was filtered, rinsed with H₂O (200 mL×5) and dried in vacuo to provide Compound 88 as a white solid (11.41 g, 81.8%).

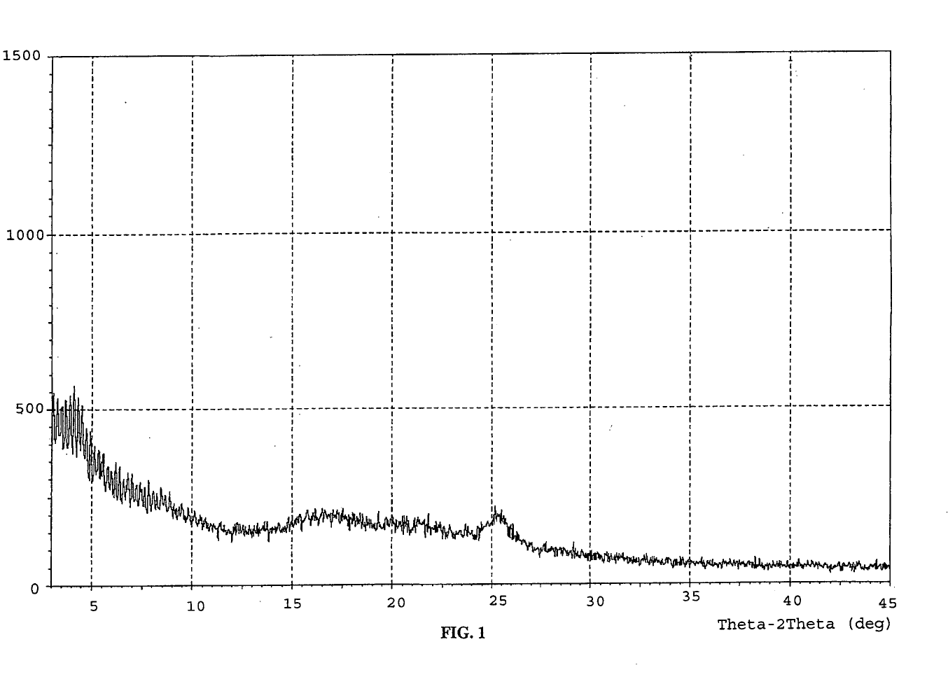

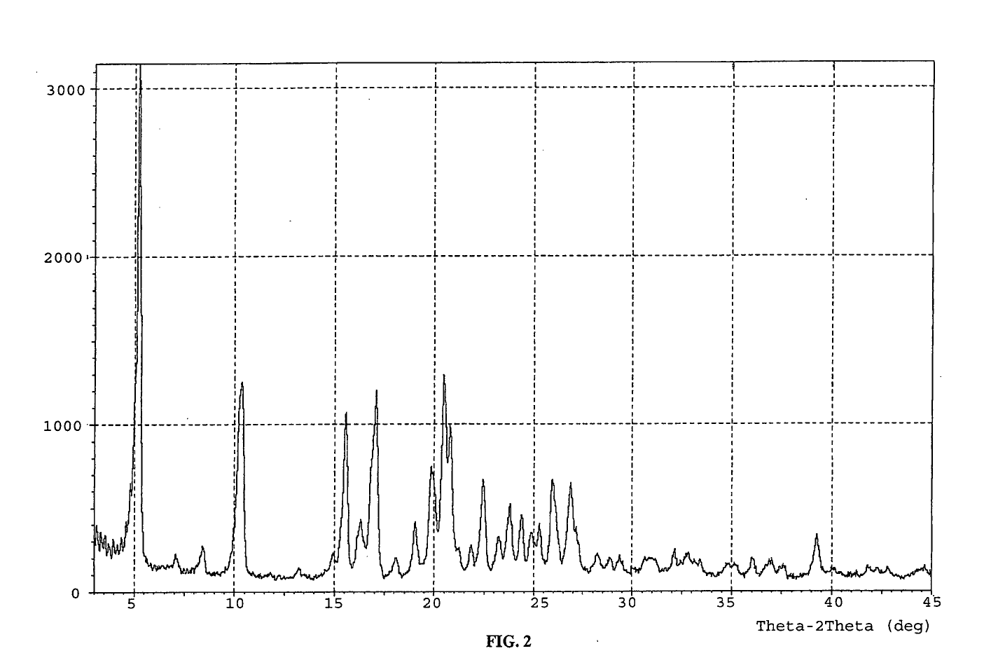

The hydrochloride salt of Compound 88 was prepared as follows. To a stirred suspension of Compound 88 (8.7 g) in ACN (175 mL) and H₂O (175 mL) was added 1N HCl (18.1 mL, 1.05 eq) affording a yellow solution. After 15 minutes, the solution was frozen on dry ice/acetone and lyophilized to provide 5-(3-(ethylsulfonyl)phenyl)-3,8-dimethyl-N-(1-methylpiperidin-4-yl)-9H-pyrido[2,3-b]indole-7-carboxamide hydrochloride as a yellow solid (9.02 g, 96.7%). The above process provided 5-(3-(ethylsulfonyl)phenyl)-3,8-dimethyl-N-(1-methylpiperidin-4-yl)-9H-pyrido[2,3-b]indole-7-carboxamide hydrochloride as the Amorphous Form, as determined by X-ray powder diffraction analysis (FIG. 1).

### Example 2

### Sample Characterization

The following analytical techniques and combination thereof were used determine the physical properties of the solid phases prepared.

1. Instrumentation

| Instrument | Vendor/Model # | AMRI # |
|---|---|---|
| Differential Scanning | Mettler 822^{e} DSC | 10667 |
| Calorimeter | | |
| Thermal Gravimetric | Mettler 851^{e} SDTA/TGA | 009111 |
| Analyzer | | |
| X-Ray Powder Diffraction | Shimadzu XRD-6000 | 009856 |
| System | | |
| Karl Fischer | Metrohm 756 KF Coulometer | 005966 |
| Nuclear Magnetic | 500 MHz Bruker AVANCE | BH-003201 |
| Resonance Spectrometer | with 5-mm BBO probe | |
| Gas Chromatograph | Agilent HP 6890 Gas | GC #9 |
| equipped with a Headspace | Chromatograph equipped with | |
| Sampler | HP 7694 Headspace Sampler | |
| Ion Chromatography | Dionex DX600 Ion | IC #1 |
| | Chromatograph | |
| High-Performance Liquid | Varian ProStar | 008956 |
| Chromatography | | |
| Moisture-Sorption Analysis | Hiden IGAsorp Moisture | IGASA030 |
| | Sorption Instrument | |

2. Differential Scanning Calorimetry Analysis (DSC)

Differential scanning calorimetry (DSC) analyses were carried out on samples weighed in an aluminum pan, covered with a pierced lid, and then crimped. Analysis conditions were 30° C. to 350° C. ramped at 10° C./min.

3. Thermal Gravimetric Analysis (TGA)

Thermal gravimetric analysis (TGA) analyses were carried out on samples weighed in an alumina crucible and analyzed from 30° C. to 230 or 250° C. and at a ramp rate of 10° C./min.

4. X-Ray Powder Diffraction (XRPD)

Samples for X-ray powder diffraction (XRPD) were placed on Si zero-return ultra-micro sample holders and analyzed using the following conditions:

| | |
|---|---|
| X-ray tube: | Cu Kα, 40 kV, 40 mA |
| Slits | |
| Divergence Slit | 1.00 deg |
| Scatter Slit | 1.00 deg |
| Receiving Slit | 0.30 mm |
| Scanning | |
| Scan Range | 3.0-45.0 deg |
| Scan Mode | Continuous |
| Step Size | 0.04° |
| Scan Rate | 2°/min |

5. Karl Fischer Analysis (KF)

Water content was determined by adding solid sample to the instrument with HYDRANAL-Coulomat AD. Micrograms of water were determined by coulometric titration.

6. Moisture-Sorption Analysis

Moisture-sorption experiments were carried out on three forms by first drying the sample at 0% RH and 25° C. until an equilibrium weight was reached or for a maximum of four hours. The sample was then subjected to an isothermal (25° C.) adsorption scan from 10 to 90% RH in steps of 10%. The sample was allowed to equilibrate to an asymptotic weight at each point for a maximum of four hours. Following adsorption, a desorption scan from 85 to 0% RH (at 25° C.) was run in steps of −10%, again allowing a maximum of four hours for equilibration to an asymptotic weight. The sample was then dried for one hour at 80° C. and the resulting solid analyzed by XRPD.

7. Nuclear Magnetic Resonance (NMR)

Samples (2 to 10 mg) were dissolved in DMSO-d₆ with 0.05% tetramethylsilane (TMS) for internal reference. ¹H-NMR spectra were acquired at 500 MHz using 5 mm broadband observe (¹H—X) Z gradient probe. A 30 degree pulse with 20 ppm spectral width, 1.0 s repetition rate, and 16 to 64 transients were utilized in acquiring the spectra.

8. Organic Volatile Impurities (OVI)

Approximately 100 mg of sample was weighed into an individual 20-mL headspace vial and 5 mL of DMSO added. The vial was then sealed and gentle shaking/vortexing was used to ensure sample was entirely dissolved. Blank samples were prepared by transferring 5.0 mL of DMSO into a 20-mL headspace vial and then sealed. Standards were prepared using stock solutions in DMSO.

Instrument Parameters were as follows:

| | |
|---|---|
| Column: | DB-1, 60 meter × 0.32 mm (inside diameter), |
| | 3-μm film thickness, P/N: 123-1064 |
| Detector: | FID; hydrogen flow of 40 mL/min, air flow of |
| | 450 mL/min. Makeup gas (helium) flow of |
| | 30 mL/min. |
| Carrier Gas: | Helium |
| Carrier Flow: | 2.2 mL/min |
| Oven Temperature: | 40° C. isothermal held for 5 minutes; ramp at |
| | 5° C./minute to |
| | 105° C.; ramp at 10° C./minute to 165° C.; ramp |
| | at 20° C./ minute to 245° C.; hold for 2 minutes |
| Injector Temperature: | 140° C. |
| Detector Temperature: | 260° C. |
| Injection Type: | Split |
| Split Flow: | 25 mL/minute (includes flow contributed by the |
| | headspace sampler) |
| Injection Volume: | 1 mL (Headspace) |
| Analysis Time: | 30 minutes |

Headspace Sampler Conditions were as follows:

| Parameter | Setting | |
|---|---|---|
| Oven Temperature: | 80° | C. |
| Loop Temperature: | 100° | C. |
| Transfer Line Temperature: | 110° | C. |
| GC Cycle Time: | 40 | minutes |
| Vial Equilibration Time: | 20 | minutes |
| Pressurization Time: | 0.13 | minutes |
| Loop Fill Time: | 0.06 | minutes |
| Loop Equilibration Time: | 0.06 | min |
| Injection Time: | 0.20 | minutes |
| Carrier Flow: | 20 | mL/min |
| Vial Pressurization: | 10.0 | psi |
| Shake: | 2 | (high) |

9. Ion Chromatography (IC)

Sample solutions in DI water were prepared with a concentration of 0.1 mg/mL. IC was performed utilizing the following conditions:

| | |
|---|---|
| Instrument: | Dionex DX600 Ion Chromatograph |
| AMRI System #: | 1 |
| Column: | Dionex IonPac AS17, 250 × 4 mm |
| Guard Column: | Dionex IonPac AS17, 50 × 4 mm |
| Column Temperature: | 35 ± 2° C. |
| Detector Operating Mode: | Suppressed Conductivity |
| Suppressor Type: | Dionex ASRS Ultra 4 mm |
| Suppressor Current: | 220 mA |
| Mobile Phase A: | Purified Water |
| Mobile Phase B: | Potassium Hydroxide, delivered using an |
| | Eluent Generator |
| Gradient: | See table below. |
| Flow Rate: | 1.5 mL/minute |
| Injection Volume: | 10 μL |
| Needle Wash: | Purified Water |
| Diluent: | Purified Water |

Gradient Conditions were as follows:

| Time | Mobile | Concentration |
|---|---|---|
| (minutes) | Phase A | of KOH (mM) |
| 0.0 | 100% | 5 |
| 3.0 | 100% | 5 |
| 10.0 | 100% | 15 |
| 20.0 | 100% | 60 |
| 20.1 | 100% | 5 |
| 30.0 | 100% | 5 |

10. High Performance Liquid Chromatography (HPLC)

Equipment used was an HPLC system equipped with a UV detector, gradient capabilities, and electronic data collection and processing, or equivalent, an autosampler capable of 10 μL injection, an analytical Column: Waters X-Terra RP18, 4.6×150 mm, 3.5 μm, P/N 186000442, an analytical balance capable of weighing to ±0.01 mg, and class A volumetric pipettes and flasks

The instrument parameters were as follows:

| | |
|---|---|
| Column: | Waters X-Terra RP18, 4.6 × 150 mm, 3.5 μm |
| Column | 45 ± 2° C. |
| Temperature: | |
| Auto-sampler | Ambient |
| Temperature: | |
| Detection: | 225 nm |
| Mobile Phase A: | 0.05% TFA in Water |
| Mobile Phase B: | 0.04% TFA in Acetonitrile |
| Gradient: | See table below |
| Flow Rate: | 1.0 mL/minute |
| Injection Volume: | 10 μL |
| Analysis Time: | 38 minutes |
| Re-equilibration Time: | 8 minutes |
| Data Collection time: | 30 minutes |
| Needle Wash: | 50:50 Acetonitrile/Water |

Gradient Conditions were as follows:

| Time (minutes) | % A | % B |
|---|---|---|
| 0.0 | 95 | 5 |
| 15.0 | 75 | 25 |
| 30.0 | 5 | 95 |
| 30.1 | 95 | 5 |
| 38.0 | 95 | 5 |

### Example 3

### Solvent Screen

A solubility study of Compound 1 in various solvents was executed to select appropriate solvents for the further crystallizations. The material was placed in vials and the solvent was added in 250 μL portions. Solvents were picked based on differences in polarity and functionality and on their classification according to the International Conference on Harmonization (ICH), with preferences given to class II and class III solvents. After each addition of solvent, the vials were visually inspected for residual solids, and further heating to 55° C. to ensure dissolution. Table 16 shows the solvents that were used and their ability to dissolve the material at room temperature.

TABLE 16
Solubility Screen of Compound 1

| | Material | | | | | |
|---|---|---|---|---|---|---|
| | Amt | Solvent Amt | Conc. | | | ICH |
| Solvent | (mg) | (mL) | (mg/mL) | Temp | Soluble | Class |
| MeCN | 2.4 | 5.25 | 0.5 | 55 | No | II |
| Dioxane | 1.1 | 1.50 | 0.7 | 55 | Yes | II |
| Acetone | 1.4 | 5.25 | 0.3 | 55 | No | III |
| MTBE | 1.2 | 4.75 | 0.3 | 55 | No | III |
| EtOH | 1.8 | 1.50 | 1.2 | 55 | Yes | III |
| EtOAc | 2.0 | 5.25 | 0.4 | 55 | No | III |
| IPAC | 2.2 | 4.75 | 0.5 | 55 | No | III |
| IPA | 2.3 | 4.75 | 0.5 | 55 | No | III |
| THF | 1.8 | 4.75 | 0.4 | 55 | No | II |
| 2-Me-THF | 2.1 | 4.75 | 0.4 | 55 | No | N/A |
| MEK | 2.8 | 4.75 | 0.6 | 55 | No | III |
| DMF | 2.6 | 0.25 | >10.4 | RT | Yes | II |
| AcOH | 2.3 | 0.25 | >9.2 | RT | Yes | III |
| MeOH | 2.5 | 0.25 | >10.0 | RT | Yes | II |
| c-Hexane | 3.1 | 6.00 | 0.5 | 55 | No | II |
| Heptane | 2.4 | 6.00 | 0.4 | 55 | No | III |
| DCM | 1.9 | 2.25 | 0.8 | RT | No | II |
| Toluene | 2.0 | 4.75 | 0.4 | 55 | No | II |
| water | 1.7 | 2.75 | 0.6 | 55 | No | N/A |
| NMP | 2.6 | 0.25 | >10.4 | RT | Yes | II |
| DMA | 2.3 | 0.25 | >9.2 | RT | Yes | II |
| Chloroform | 2.1 | 0.25 | >8.4 | RT | Yes | II |

### Example 4

### Primary and Binary Solvent Efficiency Studies with Compound 1

Solvent efficiency experiments for Compound 1 were carried out by charging the free base version of Compound 1 (15-16 mg) to an 8-Dram clear vial equipped with magnetic stir bar. Seven primary solvents (MeCN, EtOH, THF, DMA, NMP, AcOH, and DMF) were chosen based on initial solubility data obtained during the solvent screen (Table 16) and added in 100 μL portions until complete dissolution was observed with heating to 50° C. Once complete dissolution was observed HCl was added as a 1M solution (1.05 equiv.) in the reaction solvents at elevated temperatures. The resulting mixtures were then allowed to stir at that temperature for approximately 15 minutes. Four anti-solvents (MtBE, EtOAc, IPAc, and heptane) were chosen based on solubility data and added in one vol. portions at elevated temperatures until a turbid mixture was observed. Each sample was then allowed to cool to ambient temperature at a rate of 20° C./h with further stirring for 16 hours. Solids were isolated by filtration and dried under vacuum at ambient temperature for 16 hours. All samples were analyzed by XRPD with the results outlined in Tables 17-18.

TABLE 17
Solvent efficiency evaluation of Compound 1 using DMA

| Amt | DMA Amt | | | Amt Anti- | | Amt | | |
|---|---|---|---|---|---|---|---|---|
| Material | Solvent/Vol | Dissolution | Anti- | Solvent/Vol | Method of | Recovered | % | Form |
| (mg) | (mL) | Temp [° C.] | Solvent | (mL) | Isolation | (mg) | Yield | (XRPD) |
| 50.73 | 0.300/6 | n/a | — | — | n/a | n/a | n/a | n/a |
| 50.06 | 0.400/8 | 41.5 | — | — | filtration | 15.70 | 29.2 | B |
| 51.36 | 0.500/10 | 29.9 | — | — | filtration | 6.24 | 11.3 | A |
| 50.91 | 0.600/12 | 29.8 | — | — | filtration | 7.25 | 13.2 | C |
| 50.39 | 0.700/14 | 30.8 | — | — | evap. | n/a | n/a | mix |
| 50.30 | 0.500/10 | 41.0 | MTBE | 0.500/10 | filtration | 28.46 | 52.8 | B |
| 50.78 | 0.500/10 | 41.0 | MTBE | 0.250/5 | filtration | 26.53 | 48.7 | D |
| 50.31 | 0.400/8 | 41.0 | MTBE | 0.200/4 | filtration | 10.02 | 18.6 | mix |
| 52.77 | 0.450/9 | 41.0 | MTBE | 0.100/2 | filtration | 12.66 | 22.4 | B |
| 49.60 | 0.400/8 | 41.0 | MTBE | 0.100/2 | filtration | 18.38 | 34.5 | B |

TABLE 18
Solvent efficiency evaluation of Compound 1 using several organic solvents

| | | | | Amt | | | | |
|---|---|---|---|---|---|---|---|---|
| | Amt | Dissolution | | Anti-Solvent | | Amt | | |
| | Solvent Vol | Temp | Anti- | Vol | Method of | Recovered | | Form |
| Solvent | (mL/vol.) | [° C.] | Solvent | (mL/Vol) | Isolation | (mg) | % Yield | (XRPD) |
| MeCN | 2.25/150 | n/a | n/a | n/a | n/a | n/a | n/a | n/a |
| EtOH | 2.25/150 | n/a | n/a | n/a | n/a | n/a | n/a | n/a |
| THF | 2.25/150 | n/a | n/a | n/a | n/a | n/a | n/a | n/a |
| DMAc | 0.150/10 | 50 | — | — | n/a | n/a | n/a | n/a |
| DMAc | 0.150/10 | 50 | MTBE | 0.375/25 | filtered | 4.1 | 26 | D |
| DMAc | 0.150/10 | 50 | EtOAc | 0.150/10 | filtered | 2.8 | 17 | C |
| DMAc | 0.150/10 | 50 | IPAc | 0.150/10 | filtered | 1.9 | 12 | B |
| DMAc | 0.150/10 | 50 | — | — | n/a | n/a | n/a | n/a |
| NMP | 0.075/5 | 35 | — | — | filtered | 13.7 | 78 | E |
| NMP | 0.075/5 | 35 | MTBE | 0.075/5 | filtered | 2.0 | 11 | E |
| NMP | 0.075/5 | 35 | EtOAc | 0.075/5 | filtered | 9.0 | 55 | E |
| NMP | 0.075/5 | 35 | IPAc | 0.075/5 | filtered | 8.5 | 55 | E |
| NMP | 0.075/5 | 35 | heptane | 0.075/5 | filtered | 2.3 | 13 | E |
| AcOH | 0.075/10 | 25 | — | — | n/a | n/a | n/a | n/a |
| AcOH | 0.075/5 | 25 | MTBE | 0.150/10 | n/a | n/a | n/a | n/a |
| AcOH | 0.075/5 | 25 | EtOAc | 0.150/10 | n/a | n/a | n/a | n/a |
| AcOH | 0.075/5 | 25 | IPAc | 0.150/10 | n/a | n/a | n/a | n/a |
| AcOH | 0.075/5 | 25 | heptane | 0.450/30 | n/a | n/a | n/a | n/a |
| DMF | 0.225/15 | 50 | — | — | filtered | 2.0 | 13 | A |
| DMF | 0.225/15 | 50 | — | — | filtered | 4.9 | 28 | A |
| DMF | 0.225/15 | 50 | MTBE | 0.375/25 | filtered | 3.1 | 18 | A |
| DMF | 0.225/15 | 50 | EtOAc | 0.200/13 | filtered | 4.7 | 30 | A |
| DMF | 0.225/15 | 50 | IPAC | 0.200/13 | filtered | 7.7 | 44 | A |

### Example 5

### Preparation of Compound 1 from DMF/IPAc

Preparation of Compound 1 was carried out in a 250 mL 3N-RBF equipped with magnetic stir bar and thermocouple. To this was added a free base version of Compound 1 starting material (5.05 g, 0.10 mol.) followed by the portion wise (approximately 5 mL) addition of DMF (50 mL, 10 vol.) with heating to 65° C. Once complete dissolution was observed the HCl counter ion was added as a 1M solution (10.49 mL, 1.05 equiv.) in DMF at 65° C., and the resultant mixture allowed to stir for 15 min. The reaction mixture was then allowed to cool to 55° C. at a rate of 20° C./h. Once an internal temperature of 55° C. had been achieved IPAc (50 mL, 10 vol.) was added as an anti-solvent in a dropwise fashion over a 30 minute period. The reaction mixture was then further cooled to ambient temperature at the same rate (20° C./h) followed by further cooling to 0° C. with an ice/water bath. A light precipitate was observed at 30° C., and the resultant slurry was allowed to continue stirring at 0° C. for an additional 4 hours. The solids were then isolated by filtration and the filter cake dried under vacuum at 40° C. for 16 hours to give Compound 1 (3.19 g, 59% yield of Form G) as a light tan crystalline solid.

### Example 6

### Single-Solvent Crystallizations

Using the initial solubility study (Table 16) and the methods outlined below, six solvents were selected for the single solvent crystallization: MeOH, EtOH, AcOH, DMF, DMA and NMP. All solids isolated were analyzed by XRPD to determine the physical form. Table 19 shows a list of the solvents that were used and the amount of solvent needed to dissolve the material in the fast cooling procedure and Table 20 shows the same information for the slow cooling procedure. Solutions of Compound 1 in acetic acid did not form a precipitate under either slow or fast cooling conditions. Both samples were evaporated to dryness and afforded amorphous materials. XRPD analysis of non-amorphous solids showed patterns consistent with Forms A, B, C, E, G, and mixtures of Forms C and P (originally designated as Form H) as shown in Tables 19 and 20.

1. Fast-Cooling Profile

Using the initial solvent screen six solvents were selected for the single solvent crystallization: MeOH, EtOH, AcOH, DMF, DMA and NMP. Compound 1 (˜20 mg) was weighed out into vials and enough solvent (starting with 0.25 mL) was added until the material completely dissolved at elevated temperature. After hot filtration the vials were placed in a refrigerator (4° C.) for 16 hours. The resultant solids were isolated by vacuum filtration. The samples without solids were evaporated to dryness using a gentle stream of nitrogen.

All resultant solids from filtration and evaporation were dried in vacuo at room temperature and 30 inches Hg for 16 hours. All solids were analyzed by XRPD to determine the physical form. Table 19 shows a list of the solvents that were used and the amount of solvent needed to dissolve the material in the fast cooling procedure.

TABLE 19
Single solvent crystallizations of Compound 1 using fast cooling procedure

| Compound 1 | | | Temp. | | | | | |
|---|---|---|---|---|---|---|---|---|
| Amt (mg) | Solvent | Amt (mL) | (° C.) | Cooling | Precipitation | Recovery (mg) | Recovery (%) | Form |
| 20.2 | MeOH | 0.75 | 60 | Fast | Yes | 9.0 | 44.6 | C + P |
| 20.3 | EtOH | 4.00 | 75 | Fast | Yes | 12.6 | 62.1 | C + P |
| 20.4 | AcOH | 0.25 | 80 | Fast | No | n/a | n/a | amorph |
| 20.9 | DMF | 0.25 | 70 | Fast | Yes | 10.7 | 51.2 | A |
| 20.7 | DMA | 0.25 | 70 | Fast | Yes | 14.1 | 68.1 | B |
| 20.0 | NMP | 0.25 | 70 | Fast | Yes | 11.7 | 58.5 | E |

Amorph = amorphous

TABLE 20
Single solvent crystallizations of Compound 1 using slow cooling procedure

| Compound 1 | | | Temp | | | | | |
|---|---|---|---|---|---|---|---|---|
| Amt (mg) | Solvent | Amt (mL) | (° C.) | Cooling | Precipitation | Recovery (mg) | Recovery (%) | Form |
| 19.9 | MeOH | 0.5 | 60 | Slow | Yes | 11.0 | 55.3 | C |
| 20.3 | EtOH | 3.00 | 75 | Slow | Yes | 13.8 | 68.0 | C |
| 20.9 | AcOH | 0.25 | 80 | Slow | No/evap | n/a | n/a | amorph |
| 19.9 | DMF | 0.25 | 80 | Slow | Yes | 9.2 | 46.2 | G |
| 19.6 | DMA | 0.25 | 80 | Slow | Yes | 15.8 | 80.6 | B |
| 19.8 | NMP | 0.25 | 80 | Slow | Yes | 18.6 | 93.9 | E |

Amorph = amorphous

2. Slow-Cooling Profile

Using the initial solubility study six solvents were selected for the single solvent crystallization: MeOH, EtOH, AcOH, DMF, DMA and NMP. Compound 1 (approximately 20 mg) was weighed out into vials and enough solvent (starting with 0.25 mL) was added until the material completely dissolved at elevated temperature. After hot filtration the vials were slowly cooled to the room temperature at the rate of 20° C./h and stirred at this temperature for 16 hours. The resultant solids were isolated by vacuum filtration. The samples without solids were evaporated to dryness using a gentle stream of nitrogen. All resultant solids from filtration and evaporation were dried in vacuo at room temperature and 30 inches Hg for 16 hours. All solids were analyzed by XRPD to determine the physical form. Table 20 shows a list of the solvents that were used and the amount of solvent needed to dissolve the material.

### Example 7

### Binary-Solvent Crystallizations

Using the methods described below, binary solvent crystallizations were performed using MeOH, EtOH, AcOH, DMF, DMA and NMP as primary solvents. All obtained solids were analyzed by XRPD to determine the physical form. A summary of the experimental details for the fast and slow cooling experiments is presented in Tables 21 through 32.

All solids obtained from single and binary solvent crystallizations with fast and slow cooling procedures were analyzed by XRPD to determine the physical form. When either of the two known forms of the freebase (A or B) was observed, it was labeled as FB (A) or FB (B) respectively. For samples affording unique XRPD patterns, further analysis was performed on representative lots including: IC to determine counter-ion content, ¹H NMR to determine residual solvent content and confirm degradation did not occur, and thermal analysis (DSC and TGA) to characterize thermal events. Tables 36a and 36b summarize characterization data for all forms discovered in this screen. Forms A, C, and L were found to be the most common non-solvated forms observed during the screen. These materials were used for slurry, moisture sorption, and humidity chamber studies.

Water was found to be a poor crystallization anti-solvent as it afforded a free base version of Compound 1 consistently except when acetic acid was used as the primary solvent. Acetic acid was found to be a poor crystallization solvent as it often afforded amorphous solids or sticky solids which were not analyzable. Non-amorphous solids from acetic acid still showed the presence of an amorphous halo suggesting the materials were semi-crystalline, which made definitive form assignment difficult.

1. Fast-Cooling Profile

Compound 1 (approximately 20 mg) was weighed out into vials and enough primary solvent was added until the material went into solution at elevated temperature. After hot filtration antisolvent was added portion wise until the solution became turbid or the vial became full. The vials were then placed in a refrigerator (4° C.) for 16 hours. Tables 21, 23, 25, 27, 29 and 31 show experimental details. After the cooling process precipitates were isolated by filtration, they were dried in vacuo at room temperature and 30 in Hg. The vials without solids were evaporated down to dryness using a gentle stream of nitrogen and also dried in vacuo at ambient temperature and 30 in Hg. All solids were analyzed by XRPD.

2. Slow-Cooling Profile

Compound 1 (approximately 20 mg) was weighed out into vials and enough primary solvent was added until the material went into solution at elevated temperature. After hot filtration antisolvent was added portion wise until the solution became turbid or the vial became full according to the data obtained from fast-cooling experiments. The vials were then slowly cooled to room temperature at the rate of 30° C./h. Tables 22, 24, 26, 28, 30 and 32 show experimental details. After the cooling process precipitates were isolated by filtration, they were dried in vacuo at room temperature and 30 in Hg. The vials without solids were evaporated down to dryness using a gentle stream of nitrogen and also dried in vacuo at ambient temperature and 30 in Hg. All solids were analyzed by XRPD.

TABLE 21
Binary solvent crystallizations of Compound 1 using fast cooling procedure and
MeOH as primary solvent

| Compound | MeOH | Anti- | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 19.3 | 0.5 | MeCN | 6.00 | 60 | Fast | clear/ppt | 10.3 | 53.4 | C + P |
| 19.6 | 0.5 | MTBE | 0.47 | 60 | Fast | turbid/ppt | 10.6 | 54.1 | C + P |
| 20.5 | 0.5 | EtOAc | 2.00 | 60 | Fast | turbid/ppt | 13.8 | 67.3 | C + P |
| 20 | 0.5 | IPAc | 1.40 | 60 | Fast | turbid/ppt | 14.1 | 70.5 | C + P |
| 19.8 | 0.5 | IPA | 2.00 | 60 | Fast | turbid/ppt | 13.1 | 66.2 | A |
| 21.0 | 0.5 | THF | 6.00 | 60 | Fast | clear/ppt | 13.5 | 64.3 | I |
| 21.3 | 0.5 | MEK | 3.80 | 60 | Fast | turbid/ppt | 14.1 | 66.2 | A + C + P |
| 20.9 | 0.5 | Heptane | 1.00 | 60 | Fast | 2 layers/ppt | 10.5 | 50.2 | C + P |
| 20.3 | 0.5 | Water | 6.00 | 60 | Fast | clear/ppt | 1.2 | 5.9 | FB(A) |

FB(A) indicates the pattern is consistent with free base a free base versions of Compound 1.

TABLE 22
Binary solvent crystallizations of Compound 1 using slow cooling procedure and
MeOH as primary solvent

| Compound | MeOH | Anti- | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 21.5 | 0.5 | MeCN | 6.0 | 60 | Slow | clear/ppt | 14.2 | 66.0 | C |
| 20.5 | 0.5 | MTBE | 0.5 | 60 | Slow | turbid/ppt | 12.6 | 61.5 | L |
| 19.6 | 0.5 | EtOAc | 2.0 | 60 | Slow | turbid/ppt | 12.8 | 65.3 | C + P |
| 20.7 | 0.5 | IPAc | 1.4 | 60 | Slow | turbid/ppt | 14.2 | 68.6 | L |
| 20.3 | 0.5 | IPA | 2.0 | 60 | Slow | turbid/ppt | 13.3 | 65.5 | A |
| 19.8 | 0.5 | THF | 6.0 | 60 | Slow | clear/ppt | 8.4 | 42.4 | A |
| 20.8 | 0.5 | MEK | 4.0 | 60 | Slow | turbid/ppt | 14.5 | 69.7 | A |
| 19.7 | 0.5 | Heptane | 1.0 | 60 | Slow | 2 layers/ppt | 9.0 | 45.7 | L |
| 20.5 | 0.5 | Water | 6.0 | 60 | Slow | clear/ppt | 2.0 | 9.8 | FB(A) |

FB(A) indicates the pattern is consistent with free base a free base versions of Compound 1.

TABLE 23
Binary solvent crystallizations of Compound 1 using fast cooling procedure and
EtOH as primary solvent

| Compound | EtOH | Anti | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 21.9 | 4.0 | MeCN | 15 | 75 | Fast | clear/no ppt | 10.8 | 49.3 | C + P |
| 20.7 | 3.0 | MTBE | 7 | 70 | Fast | turbid/ppt | 17 | 82.1 | J |
| 21.5 | 3.0 | EtOAc | 15 | 70 | Fast | clear/ppt | 14.3 | 66.5 | A + C + P |
| 20.5 | 3.0 | IPAc | 15 | 70 | Fast | clear/ppt | 14.2 | 69.3 | A + C + P |
| 20.6 | 3.0 | IPA | 15 | 70 | Fast | clear/ppt | 15.4 | 74.8 | J |
| 20.7 | 3.0 | THF | 15 | 70 | Fast | clear/no ppt | 9.6 | 46.4 | K |
| 20.1 | 3.0 | MEK | 15 | 70 | Fast | clear/ppt | 10.5 | 52.2 | A |
| 20.3 | 3.0 | Heptane | 5 | 70 | Fast | turbid/ppt | 16.8 | 82.8 | C + P |
| 21.9 | 3.0 | Water | 15 | 70 | Fast | clear/ppt | 4.3 | 19.6 | FB(A) |

FB(A) indicates the pattern is consistent with free base a free base versions of Compound 1.

TABLE 24
Binary solvent crystallizations of Compound 1 using slow cooling procedure and
EtOH as primary solvent

| Compound | EtOH | Anti- | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 20.2 | 3.0 | MeCN | 15 | 70 | Slow | clear/no ppt | 10.8 | 53.5 | C + P |
| 19.6 | 3.0 | MTBE | 7 | 70 | Slow | turbid/ppt | 16.2 | 82.7 | L |
| 19.9 | 3.0 | EtOAc | 15 | 70 | Slow | clear/ppt | 14 | 70.4 | C + P |
| 21.3 | 3.0 | IPAc | 15 | 70 | Slow | clear/ppt | 17.1 | 80.3 | A |
| 20.8 | 3.0 | IPA | 15 | 70 | Slow | clear/ppt | 11.6 | 55.8 | C + P |
| 20.2 | 3.0 | THF | 15 | 70 | Slow | clear/ppt | 5.6 | 27.7 | C + P |
| 21.5 | 3.0 | MEK | 15 | 70 | Slow | clear/ppt | 14.1 | 65.6 | A |
| 20.4 | 3.0 | Heptane | 5 | 70 | Slow | turbid/ppt | 16.7 | 81.9 | A + C + P |
| 20.7 | 3.0 | Water | 15 | 70 | Slow | clear/ppt | 4.0 | 19.3 | FB(A) |

FB(A) indicates the pattern is consistent with free base a free base versions of Compound 1.

TABLE 25
Binary solvent crystallizations of Compound 1 using fast cooling procedure and
AcOH as primary solvent

| Compound | AcOH | Anti- | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 19 | 0.25 | MeCN | 7.00 | 70 | Fast | Clear/No | n/a | n/a | amorph |
| 19.8 | 0.25 | MTBE | 0.25 | 70 | Fast | Turbid/Yes | 4.5 | 22.7 | amorph |
| 19.5 | 0.25 | EtOAc | 6.00 | 70 | Fast | Turbid/Yes | 8.8 | 45.1 | amorph |
| 19.1 | 0.25 | IPAc | 1.35 | 70 | Fast | Turbid/Yes | 3.7 | 19.4 | amorph |
| 20.7 | 0.25 | IPA | 7.00 | 70 | Fast | Clear/Yes | 9.7 | 46.9 | C + P + J |
| 19.3 | 0.25 | THF | 7.00 | 70 | Fast | Clear/Yes | 12.3 | 63.7 | I |
| 19.8 | 0.25 | MEK | 7.00 | 70 | Fast | Clear/Yes | 8.9 | 44.9 | L |
| 20.6 | 0.25 | Heptane | 0.30 | 70 | Fast | 2 layers/No | n/a | n/a | amorph |
| 19.8 | 0.25 | Water | 7.00 | 70 | Fast | Clear/No | n/a | n/a | n/a |

Amorph = amorphous
n/a Indicates the sample was not an isolatable sol

TABLE 26
Binary solvent crystallizations of Compound 1 using slow cooling procedure and
AcOH as primary solvent

| Compound | AcOH | Anti- | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 19.7 | 0.25 | MeCN | 7.00 | 70 | Slow | Clear/Yes | 9.5 | 48.2 | C |
| 20.1 | 0.25 | MTBE | 0.30 | 70 | Slow | Turbid/Yes | 9.9 | 49.3 | J |
| 20.5 | 0.25 | EtOAc | 6.00 | 70 | Slow | Turbid/Yes | 13 | 63.4 | J |
| 21.2 | 0.25 | IPAc | 1.35 | 70 | Slow | Turbid/Yes | 12.5 | 59.0 | J |
| 21 | 0.25 | IPA | 7.00 | 70 | Slow | Clear/Yes | 11.2 | 53.3 | J w/ add |
| 20.0 | 0.25 | THF | 7.00 | 70 | Slow | Clear/Yes | 11.7 | 58.5 | J w/ add |
| 20.6 | 0.25 | MEK | 7.00 | 70 | Slow | Clear/Yes | 12.4 | 60.2 | L |
| 19.7 | 0.25 | Heptane | 0.20 | 70 | Slow | 2 layers/No | n/a | n/a | amorph |
| 20.0 | 0.25 | Water | 7.00 | 70 | Slow | Clear/No | n/a | n/a | n/a |

Amorph = amorphous
J w/add = Form J with additional peaks present. Due to the semi-crystalline nature of the material definitive determination was not possible.
n/a Indicates the sample was not an isolatable solid

TABLE 27
Binary solvent crystallizations of Compound 1 using fast cooling procedure and
DMF as primary solvent

| Compound | DMF | Anti- | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 20.2 | 0.25 | MeCN | 0.77 | 70 | Fast | Turbid/Yes | 16.1 | 79.7 | A |
| 21.2 | 0.25 | MTBE | 0.40 | 70 | Fast | Turbid/Yes | 12.8 | 60.4 | A |
| 19.2 | 0.25 | EtOAc | 0.38 | 70 | Fast | Turbid/Yes | 13.0 | 67.7 | A |
| 19.8 | 0.25 | IPAc | 0.20 | 70 | Fast | Turbid/Yes | 15.7 | 79.3 | G + A |
| 19.7 | 0.25 | IPA | 0.92 | 70 | Fast | Turbid/Yes | 13.8 | 70.1 | A |
| 20.9 | 0.25 | THF | 0.97 | 70 | Fast | Turbid/Yes | 19.1 | 91.4 | I |
| 20.6 | 0.25 | MEK | 0.63 | 70 | Fast | Turbid/Yes | 17.2 | 83.5 | G + A |
| 19.6 | 0.25 | Heptane | 1.00 | 70 | Fast | 2 layers/yes | 11.0 | 56.1 | G |
| 20.6 | 0.25 | Water | 6.00 | 70 | Fast | Turbid/Small | 1.4 | 6.8 | FB(A) |

FB(A) indicates the pattern is consistent with free base a free base versions of Compound 1.

TABLE 28
Binary solvent crystallizations of Compound 1 using slow cooling procedure and
DMF as primary solvent

| Compound | DMF | Anti- | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 20.9 | 0.25 | MeCN | 0.60 | 70 | Slow | Turbid/Yes | 16.1 | 77.0 | C |
| 19.7 | 0.25 | MTBE | 0.39 | 70 | Slow | Turbid/Yes | 12.8 | 65.0 | A + G |
| 19.1 | 0.25 | EtOAc | 0.50 | 70 | Slow | Turbid/Yes | 13.0 | 68.1 | A |
| 20.5 | 0.25 | IPAc | 0.15 | 70 | Slow | Turbid/Yes | 15.7 | 76.6 | A + G |
| 20.4 | 0.25 | IPA | 0.96 | 70 | Slow | Turbid/Yes | 13.8 | 67.6 | C + P |
| 19.6 | 0.25 | THF | 1.00 | 70 | Slow | Turbid/Yes | 19.1 | 97.4 | I |
| 20.3 | 0.25 | MEK | 0.82 | 70 | Slow | Turbid/Yes | 17.2 | 84.7 | G |
| 20.9 | 0.25 | Heptane | 1.00 | 70 | Slow | 2 layers/Yes | 11.0 | 52.6 | G |
| 20.0 | 0.25 | Water | 6.00 | 70 | Slow | Turbid/Small | 1.4 | 7.0 | FB(A) |

FB(A) indicates the pattern is consistent with free base a free base versions of Compound 1.

TABLE 29
Binary solvent crystallizations of Compound 1 using fast cooling procedure and
DMA as primary solvent

| Compound | DMA | Anti- | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 19.5 | 0.25 | MeCN | 0.60 | 70 | Fast | Turbid/Yes | 15.7 | 80.5 | A |
| 20.7 | 0.25 | MTBE | 0.30 | 70 | Fast | Turbid/Yes | 16 | 77.3 | B |
| 20.2 | 0.25 | EtOAc | 0.45 | 70 | Fast | Turbid/Yes | 17.1 | 84.7 | B |
| 19.9 | 0.25 | IPAc | 0.32 | 70 | Fast | Turbid/Yes | 17.8 | 89.4 | B |
| 19.9 | 0.25 | IPA | 0.60 | 70 | Fast | Turbid/Yes | 15.6 | 78.4 | A |
| 19.5 | 0.25 | THF | 0.60 | 70 | Fast | Turbid/Yes | 16.8 | 86.2 | B + A |
| 20.8 | 0.25 | MEK | 0.65 | 70 | Fast | Turbid/Yes | 17.8 | 85.6 | B + A |
| 20.1 | 0.25 | Heptane | 0.22 | 70 | Fast | 2 layers/Yes | 14.8 | 73.6 | B |
| 20.0 | 0.25 | Water | 6.00 | 70 | Fast | Clear/Small | 3.6 | 18.0 | FB(A) |

FB(A) indicates the pattern is consistent with free base a free base versions of Compound 1.

TABLE 30
Binary solvent crystallizations of Compound 1 using slow cooling procedure and
DMA as primary solvent

| Compound | DMA | Anti- | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 19.7 | 0.25 | MeCN | 0.51 | 70 | Slow | Turbid/Yes | 10.0 | 50.8 | C |
| 19.2 | 0.25 | MTBE | 0.27 | 70 | Slow | Turbid/Yes | 16.5 | 85.9 | B + A |
| 20.7 | 0.25 | EtOAc | 0.45 | 70 | Slow | Turbid/Yes | 18.9 | 91.3 | B + A |
| 20.5 | 0.25 | IPAc | 0.35 | 70 | Slow | Turbid/Yes | 17.9 | 87.3 | B + A |
| 20.4 | 0.25 | IPA | 0.72 | 70 | Slow | Turbid/Yes | 16.1 | 78.9 | A |
| 20.4 | 0.25 | THF | 0.75 | 70 | Slow | Turbid/Yes | 19.2 | 94.1 | B + A |
| 20.5 | 0.25 | MEK | 0.73 | 70 | Slow | Turbid/Yes | 18.9 | 92.2 | B + A |
| 20.5 | 0.25 | Heptane | 0.25 | 70 | Slow | 2 layers/Yes | 14.8 | 72.2 | B |
| 20.6 | 0.25 | Water | 6.00 | 70 | Slow | Clear/Small | 1.0 | 4.9 | FB(A) |

FB(A) indicates the pattern is consistent with free base a free base versions of Compound 1.

TABLE 31
Binary solvent crystallizations of Compound 1 using fast cooling procedure and
NMP as primary solvent

| Compound | NMP | Anti- | Amt | Temp. | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 19.4 | 0.25 | MeCN | 1.00 | 70 | Fast | Turbid/Yes | 17.7 | 91.2 | E |
| 20 | 0.25 | MTBE | 0.43 | 70 | Fast | Turbid/Yes | 16.1 | 80.5 | E |
| 20.6 | 0.25 | EtOAc | 0.65 | 70 | Fast | Turbid/Yes | 18.3 | 88.8 | E |
| 20.5 | 0.25 | IPAc | 0.50 | 70 | Fast | Turbid/Yes | 19.4 | 94.6 | E |
| 19.8 | 0.25 | IPA | 6.00 | 70 | Fast | Clear/Yes | 15.1 | 76.3 | A |
| 19.2 | 0.25 | THF | 2.00 | 70 | Fast | Turbid/Yes | 14.9 | 77.6 | E |
| 20.8 | 0.25 | MEK | 1.00 | 70 | Fast | Turbid/Yes | 19.4 | 93.3 | E |
| 20.7 | 0.25 | Heptane | 0.70 | 70 | Fast | 2 layers/Yes | 18.0 | 87.0 | E |
| 20.9 | 0.25 | Water | 6.00 | 70 | Fast | Clear/Small | <1 | n/a | n/a |

N/A - sample was not analyzable.

TABLE 32
Binary solvent crystallizations of Compound 1 using slow cooling procedure and
NMP as primary solvent

| Compound | NMP | Anti- | Amt | Temp | | | Recovery | Recovery | |
|---|---|---|---|---|---|---|---|---|---|
| 1 (mg) | (mL) | Solvent | (mL) | (° C.) | Cooling | Precipitation | (mg) | (%) | Form |
| 20.3 | 0.25 | MeCN | 1.00 | 70 | Slow | Turbid/Yes | 18.8 | 92.6 | E |
| 19.8 | 0.25 | MTBE | 0.42 | 70 | Slow | Turbid/Yes | 18.6 | 93.9 | E |
| 19.2 | 0.25 | EtOAc | 0.67 | 70 | Slow | Turbid/Yes | 18.7 | 97.4 | E |
| 20.3 | 0.25 | IPAc | 0.50 | 70 | Slow | Turbid/Yes | 18.5 | 91.1 | E |
| 20.2 | 0.25 | IPA | 6.00 | 70 | Slow | Clear/Yes | 16.2 | 80.2 | A |
| 19.2 | 0.25 | THF | 2.00 | 70 | Slow | Turbid/Yes | 19.6 | 102.1 | I |
| 19.4 | 0.25 | MEK | 1.00 | 70 | Slow | Turbid/Yes | 18.8 | 96.9 | E |
| 20.9 | 0.25 | Heptane | 0.70 | 70 | Slow | 2 layers/Yes | 15.8 | 75.6 | E |
| 20.7 | 0.25 | Water | 6.00 | 70 | Slow | Clear/Small | 1.7 | 8.2 | FB(A) |

FB(A) indicates the pattern is consistent with free base a free base versions of Compound 1.

TABLE 33
Details of scale-up experiments of selected forms

| Targeted | Compound | | Amount | Anti- | Amount | Temp | | Recovery | Obtained |
|---|---|---|---|---|---|---|---|---|---|
| Form | 1 (mg) | Solvent | (mL) | Solvent | (mL) | (° C.) | Cooling | (%) | Form |
| A | 300 | DMA | 3.60 | IPA | 10.50 | 70 | Slow | 95.3 | N |
| C | 301 | DMF | 3.60 | MeCN | 10.00 | 70 | Slow | 91.7 | L |
| H* | 301 | MeOH | 9.50 | EtOAc | 30.00 | 60 | Slow | 75.7 | N |
| A | 400 | MeOH | 10 | IPA | 40 | 60 | Slow | 84.00 | C + P* |
| C | 400 | MeOH | 10 | MeCN | 112 | 60 | Slow | 76.05 | C |
| H* | 400 | EtOH | 60 | EtOAc | 300 | 70 | Slow | 80.33 | C + P* |

*A mixture of Forms C and P.

TABLE 34
Details and results of 1 week slurry experiments

| | Starting | | | | | | | |
|---|---|---|---|---|---|---|---|---|
| | material | Amt | | | | Recovery | Recovery | Form |
| Entry | (Form) | (mg) | Solvent | Amt (mL) | Temp (° C.) | (mg) | (%) | (1 wk) |
| 1 | Form N | 34.1 | water | 1.0 | RT | 23.0 | 67.4 | A |
| 2 | | 28.8 | IPA | 1.0 | RT | 21.2 | 73.6 | C + P |
| 3 | | 27.9 | EtOAc | 1.0 | RT | 23.3 | 83.5 | C + P |
| 4 | | 27.5 | MeCN | 1.0 | RT | 23.2 | 84.4 | C |
| 5 | | 27.6 | EtOH | 1.0 | RT | 21.7 | 78.6 | C |
| 6 | | 27.1 | Dioxane | 1.0 | RT | 24.1 | 88.9 | C + P |
| 7 | Form L | 30.3 | water | 1.0 | RT | 16.4 | 54.1 | A |
| 8 | | 26.8 | IPA | 1.0 | RT | 23.9 | 89.2 | L |
| 9 | | 27.7 | EtOAc | 1.0 | RT | 25.3 | 91.3 | L |
| 10 | | 27.5 | MeCN | 1.0 | RT | 18.6 | 67.6 | C |
| 11 | | 27.2 | EtOH | 1.0 | RT | 20.8 | 76.5 | C |
| 12 | | 28.2 | Dioxane | 1.0 | RT | 25.0 | 88.7 | L |
| 13 | Form N | 30.3 | water | 1.0 | RT | 21.1 | 69.6 | A |
| 14 | | 30.1 | IPA | 1.0 | RT | 24.7 | 82.1 | C |
| 15 | | 27.7 | EtOAc | 1.0 | RT | 23.1 | 83.4 | C |
| 16 | | 27.5 | MeCN | 1.0 | RT | 18.1 | 65.8 | C |
| 17 | | 28.7 | EtOH | 1.0 | RT | 24.1 | 84.0 | C |
| 18 | | 26.4 | Dioxane | 1.0 | RT | 19.8 | 75.0 | C |
| 19 | Form C | 27.1 | water | 1.0 | RT | 22.3 | 82.3 | A |
| 20 | | 27.3 | IPA | 1.0 | RT | 23.6 | 86.4 | C |
| 21 | | 28.4 | EtOAc | 1.0 | RT | 24.5 | 86.3 | C |
| 22 | | 27.9 | MeCN | 1.0 | RT | 25.6 | 91.8 | C |
| 23 | | 28.0 | EtOH | 1.0 | RT | 23.9 | 85.4 | C |
| 24 | | 27.5 | Dioxane | 1.0 | RT | 22.5 | 81.8 | C |
| 25 | Form H | 26.8 | water | 1.0 | RT | 18.8 | 70.1 | A |
| 26 | | 26.8 | IPA | 1.0 | RT | 23.5 | 87.7 | C |
| 27 | | 26.8 | EtOAc | 1.0 | RT | 24.8 | 92.5 | C |
| 28 | | 27.6 | MeCN | 1.0 | RT | 24.5 | 88.8 | C |
| 29 | | 26.5 | EtOH | 1.0 | RT | 21.8 | 82.3 | C |
| 30 | | 26.5 | Dioxane | 1.0 | RT | 22.2 | 83.8 | C |
| 31 | Form A | 26.6 | water | 1.0 | RT | 19.2 | 72.2 | A |
| 32 | | 29.7 | IPA | 1.0 | RT | 20.9 | 70.3 | A |
| 33 | | 29.2 | EtOAc | 1.0 | RT | 10.7 | 36.6 | A |
| 34 | | 27.4 | MeCN | 1.0 | RT | 12.9 | 47.1 | C |
| 35 | | 26.8 | EtOH | 1.0 | RT | 17.0 | 63.4 | C |
| 36 | | 28.2 | Dioxane | 1.0 | RT | 17.6 | 62.4 | A |

TABLE 35
Details and results of MeCN/water slurry experiments of Forms A and C at
ambient temperature.

| | Amt | Amt | MeCN/water | | | Filtration | | Filtration |
|---|---|---|---|---|---|---|---|---|
| Starting | Form A | Form C | (v/v | Amt | Form | after | Form | after |
| material | (mg) | (mg) | ratio) | (mL) | 1 day | 1 day | 1 wk | 1 week |
| 50:50 A/C | 20.2 | 20.0 | 1:9 | 2.0 | A | slow | A | slow |
| | 20.8 | 20.4 | 1:1* | 2.0 | * | * | * | * |
| | 20.5 | 20.8 | 9:1 | 2.0 | A | fast | A | fast |
| Form A | 39.4 | 0.0 | 1:9 | 2.0 | A | slow | A | slow |
| | 37.2 | 0.0 | 1:1* | 2.0 | * | * | * | * |
| | 38.0 | 0.0 | 9:1 | 2.0 | A | fast | A | fast |
| Form C | 0.0 | 40.2 | 1:9 | 2.0 | A | slow | A | slow |
| | 0.0 | 37.8 | 1:1* | 2.0 | * | * | * | * |
| | 0.0 | 39.2 | 9:1 | 2.0 | A | fast | A | fast |

*All solids were dissolved in 50/50 mixtures of MeCN/water, therefore characterization was not possible.

TABLE 36a
Analytical results summary for non-solvated forms of Compound 1.

| | | | | TGA | | |
|---|---|---|---|---|---|---|
| Form | State | Solvents/Conditions | DSC (° C.) | (wt %) | IC | NMR |
| A | Monohydrate | Slurry in water | 68, 198\hat{ }, 327 | 2.4 | 1.0:1 | Consistent; |
| | | | | | | Protonated |
| | | | | | | species observed |
| C | Anhydrate | MeOH/MeCN | 335 | 0.0 | 1.0:1 | Consistent; |
| | | | | | | Protonated |
| | | | | | | species observed |
| D | Anhydrate | DMA/MTBE | 249, 264\hat{ }, 318 | 1.8 | 0.6:1 | Consistent; |
| | | (observed twice) | | | | Protonated |
| | | | | | | species observed |
| F | De-solvate | Heat solvate in | 328 | — | 1.0:1 | Consistent; |
| | | TGA | | | | Protonated |
| | | | | | | species observed |
| H | Anhydrate | Fast cooling and | 331 | 0.5 | 1.0:1 | Consistent; |
| (C + P) | | slow cooling with | | | | Protonated |
| | | multiple solvents | | | | species observed |
| J | Anhydrate | Fast cooling and | 219, 225\hat{ }, 236\hat{ }, | 0.4 | 0.9:1 | Consistent; |
| | | slow cooling with | 323, 328, 338 | | | Protonated |
| | | multiple solvents | | | | species observed |
| K | Anhydrate | EtOH/THF fast | 322 | 0.0 | 0.9:1 | Consistent; |
| | | cooling | | | | Protonated |
| | | | | | | species observed |
| L | Channel | EtOH/MTBE, | 333 | 1.7 | 1.0:1 | Consistent; |
| | hydrate | DMF/MeCN | | | | Protonated |
| | | | | | | species observed |
| M | unknown hydrate | DMF/IPAc | 215, 332 | 6.0 | — | Consistent; |
| | | | | | | Protonated |
| | | | | | | species observed |
| N | unknown hydrate | MeOH/EtOAc | 335** | 2.4 | 1.0:1* | Consistent; |
| | | | | | | Protonated |
| | | | | | | species observed |
| O | De-hydrate | Heat hydrate in | 332 | — | — | — |
| | | TGA | | | | |
| P | Unknown form | Found during in- | — | — | — | — |
| | (metastable) | process checks that | | | | |
| | | yielded Form H | | | | |

\hat{ }Indicates exothermic event;
*Takes into account one-half mole of water present;
**Baseline shift observed at ~175° C.;
— indicates test was not performed or does not apply

TABLE 36b
Analytical results summary for solvate forms of Compound 1.

| | | | | TGA | | |
|---|---|---|---|---|---|---|
| Form | State | Solvents/Conditions | DSC (° C.) | (wt %) | IC | NMR |
| B | DMA Solvate | DMA as primary | 189, 238\hat{ }, 330 | 11.7 | 0.8:1* | Consistent; |
| | | solvent | | | | Protonated species observed |
| E | NMP Solvate | NMP as primary | 220, 228\hat{ }, 236 | 11.9 | 0.8:1* | Consistent; |
| | | solvent | | | | Protonated species observed |
| G | DMF Solvate | DMF as primary | 201, 336 | 11.6 | 1.2:1* | Consistent; |
| | | solvent | | | | Protonated species observed |
| I | THF Solvate | THF used as anti- | 206, 242\hat{ }, 336 | 7.9 | 0.9:1** | Consistent; |
| | | solvent | | | | Protonated species observed |

\hat{ }Indicates exothermic event.
*Takes one mole of solvent into account.
**Takes 6.4 wt % THF present into account - form may be a hemi-solvate or incomplete solvate.

### Example 8

### Scale-Up Experiments of Compound 1, Forms A, C and L

Preparation of Forms A, C, and H of Compound 1 were carried out on 300 mg scale in DMA/IPA, DMF/MeCN, and MeOH/EtOAc respectively, by charging Compound 1 to a 25 mL equipped with magnetic stir bar and thermocouple. To this was added the appropriate solvent in one portion followed by heating to 60-70° C. with stirring until complete dissolution was observed. Each reaction solution was polish filtered followed by the addition of anti-solvents at elevated temperatures with additional stirring for 5-10 minutes. Each reaction was then allowed to slowly cool to ambient temperature at a rate of 20° C./h, followed by further stirring for 16 hours. Solids were isolated by filtration and dried under vacuum at ambient temperature for 16 hours to give Compound 1 (286 mg, 95% yield; 276 mg, 91% yield; and 228 mg, 75% yield) as Forms N, L, and N respectively. A summary of the experimental details are outlined in Table 33.

Preparation of Forms A, C and H of Compound 1 were also carried out on 400 mg scale in MeOH/IPA, MeOH/MeCN and EtOH/EtOAc, respectively. Form C was produced by using MeOH instead of DMF as the primary solvent. The crystallization targeting Form A was found to produce a mixture of Forms C and P (originally designated as Form H) upon isolation. In-process checks presented a pattern consistent with Form P. Since the mixture of forms was isolated, the material was seeded with Form A in an effort to generate Form A. However, seeding did not produce Form A. Therefore the resulting material was isolated as a mixture and then was further slurried in water to afford Form A.

### Example 9

### Slurry Experiments

Slurry experiments were performed using six solvents (water, IPA, EtOAc, MeCN, EtOH and dioxane) and six lots of Compound 1. Each vial was charged with approximately 25-30 mg of API and 1 mL of the corresponding solvent, followed by stirring at room temperature for one week. Table 34 shows experimental details. The obtained solids were isolated by filtration, dried under vacuum at room temperature and analyzed by XRPD.

Slurry experiments of Compound 1, Forms A and C, were conducted in mixtures of MeCN and water. Each vial was charged with approximately 40 mg of Compound 1 (pure Form A, pure Form C, or 50:50 mixtures) and 2 mL of MeCN/water mixture in a 1:9, 1:1, and 9:1 ratio. Table 35 provides the experimental details. Some samples (indicated in table) afforded clear solutions in the 1:1 mixture of MeCN/water, therefore characterization of these samples could not be performed, but the experiment was continued to determine if a more stable form would precipitate. All samples were stirred at room temperature for approximately 24 hours. An aliquot (1 mL) of the suspension was taken from the slurries (samples #1, 3, 4, 6, 7 and 9) for determination of form by XRPD. All aliquots were filtered and dried in vacuo at 30 mm Hg and ambient temperature. All samples were stirred at ambient temperature for an additional six days, filtered, dried, and again the solid form and filterability were determined.

It was also found during the concurrent process control screen that Form A was isolated when Form B or Form G was slurried in DI water for several hours (2-24 hours).

### Example 10

### Further Preparation of Compound 1, Forms A and C

Preparation of the Compound 1 Form A was carried out by charging a free base version of Compound 1 (18 g, 0.036 mol.) to a 1 L-3N RBF equipped with magnetic stir bar and thermocouple. To this was added a 1:1 (v/v) mixture of MeCN and water in one portion at ambient temperature. Once complete dissolution was observed, concentrated HCl (3.062 mL, 1.05 equiv.) was added to the reaction solution, followed by additional stirring for 10 minutes, followed by a polish filtration. Additional MeCN (720 mL, 40 vol.) was then added as an anti-solvent to facilitate precipitation. The resultant slurry was allowed to stir at ambient temperature for approximately 19 h, before the solids were isolated by filtration. The filter cake was rinsed with two portions of MeCN (2×100 mL, 5.6 vol.), and dried under vacuum at ambient temperature for 16 hours to give Compound 1 (16.19 g, 84% yield) as a light orange crystalline (Form A) powder.

Compound 1 Form A (5.05 g) was slurried in 50 mL (10 vol.) of anhydrous MeCN at room temperature under nitrogen. In-process samples were taken after 24, 72, and 96 hours and XRPD showed the material to still be Form A. To help promote conversion to Form C, the reaction mixture was diluted with anhydrous MeCN (150 mL, 30 vol.) and allowed to stir at room temperature for three days, as previous experiments to convert Form A to Form C were successful using 30 or more volumes of MeCN. After stirring for an additional three days the material was still found to be Form A.

Based on a previous observation that Form C could also be obtained from a cooling crystallization employing acetonitrile and MeOH, it was decided to add anhydrous MeOH (50 mL, 10 volumes) and to heat the reaction mixture to 60° C. in order to improve the solubility of the material and possibly promote solution-mediated polymorph conversion. After stirring with the addition of MeOH, the slurry visibly thinned out then thickened after approximately 30 minutes at 60° C. Analysis of a small aliquot from the batch by XRPD showed the material to be consistent with Form C. After stirring the slurry at 60° C. for a total of 1.5 hours, the mixture was naturally cooled to room temperature. The solids were isolated by filtration, washed with MeCN (2×50 mL) and dried under vacuum at 40° C. to afford 4.26 g of Compound 1 Form C material (84% recovery).

### Example 11

### Solubility Study

The solubility measurement of Compound 1 Form A was performed in DI water (Milli-Q) and in 20 mM phosphate buffer (pH 3.2), see Table 37. Each vial was charged with 50-55 mg of Compound 1 and 1 mL of the corresponding solvent.

TABLE 37
Solubility of Form A of Compound 1.

| | | Solu- | | | | |
|---|---|---|---|---|---|---|
| | | bility | | Solubility | pH | |
| | pH | mg/mL | XRPD | mg/mL | (final, | XRPD |
| Media | (initial) | (1 day) | (1 day) | (1 week) | 1 week) | (1 week) |
| DI water | 8.1 | 3.1 | Form A | 4.3 | 5.0 | Form A |
| Phosphate | 3.2 | 3.3 | Form A | 4.1 | 3.3 | Form A |
| buffer | | | | | | |

Both slurries were stirred at room temperature for approximately 19 hours. An aliquot (0.2 mL) was taken from each sample for the solubility determination and XRPD test. Table 36 shows the results of these tests. Both slurries were allowed to stir for an additional 6 days at room temperature. The pH of the supernatant liquid and solubility were determined following the 1 week slurry.

### Example 12

### Humidity Chamber Study

Humidity chamber studies of Compound 1 Forms A and C were set up at ambient temperature as shown in Table 38. The 0% RH chamber was prepared with drierite and the 95% RH chamber was made with a saturated solution of Na₂HPO₄.12H₂O in DI water. The chambers were equilibrated for >1 week prior to introducing samples. Samples were placed in Teflon-lined caps for scintillation vials and allowed to equilibrate for one week then analyzed by XRPD and KF to determine form and water content.

TABLE 38
Humidity chamber studies of Compound 1 Forms A and C.

| Form | KF | | Result Form | Result |
|---|---|---|---|---|
| (initial) | (initial) | % RH | (1 week) | KF (1 week) |
| C | 0.20 | 0% | C | 0.51 |
| A | 3.3 | 0% | A | 3.6 |
| C | 0.20 | 95% | A | 3.5 |
| A | 3.3 | 95% | A | 3.3 |

Further stability studies in humid environments were performed and showed Form C converted to Form A after equilibrating at 95% RH for one week at ambient temperature. FIG. 60 summarizes the conversion of the forms of Compound 1 observed from slurry and humidity chamber studies.

### Example 13

### Drying Study

Compound 1 (4.08 g, Form A) was dried under vacuum at 40° C. overnight. A small sample (˜200 mg) was taken for XRPD and OVI tests. The rest of the material was further dried under vacuum at 55° C. overnight. A second sample (˜200 mg) was taken for XRPD and OVI tests. The oven temperature was increased to 70° C. and the material was further dried under vacuum at 70° C. overnight. A third sample (˜200 mg) was taken for XRPD and OVI tests. The oven temperature was then increased to 85° C. and the material was further dried under vacuum over weekend. Analysis by XRPD and OVI was completed as shown in Table 39 along with experimental details. A summary of moisture sorption data for Forms A, C, and L is shown in Table 40.

TABLE 39
Drying Study of Compound 1 Form A

| Temperature | Time at Temperature | Form by | Residual Amt of |
|---|---|---|---|
| (° C.) | (total) | XRPD | MeCN by OVI (ppm) |
| 40 | 16 | A | 1353 |
| 55 | 24 (40) | A | 1303 |
| 70 | 24 (64) | A | 1113 |
| 85 | 72 (136) | A | 670 |

TABLE 40
Summary of Moisture Data for Forms A, C, and L

| Form | State | KF | Moisture Sorption | |
|---|---|---|---|---|
| A | Monohydrate | 3.1%* | 60% RH: | 3.7% |
| | | | 90% RH: | 4.2% |

XRPD consistent
before and after
analysis

| | | | | |
|---|---|---|---|---|
| C | Anhydrate | 0.2% | 60% RH: | 1.4% |
| | | | 90% RH: | 1.9% |

XRPD consistent
before and after
analysis

| | | | | |
|---|---|---|---|---|
| L | Channel | 2.1% | 60% RH: | 2.9% |
| | Hydrate | | 90% RH: | 3.9% |

The theoretical weight % water for a monohydrate is 3.2%.

What is claimed is:

1. A polymorphic form of Compound 1 having the formula: 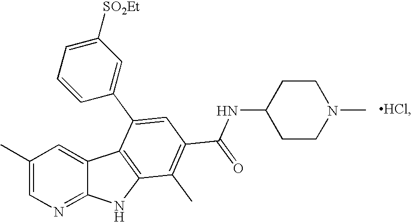
wherein the polymorphic form is selected from the group consisting of Form B, Form C, Form D, Form E, Form F, Form G, Form I, Form J, Form K, Form L, Form M, Form N, Form O and Form P.

2. The polymorphic form of claim 1, wherein the polymorphic form is Form B which is a dimethylacetamide (DMA) solvate having an X-ray powder diffraction pattern (CuKα) comprising significant diffraction peaks at about 13.8, 17.1 and 19.7 degrees 2-theta (°2θ).

3. The polymorphic form of claim 2, wherein the X-ray powder diffraction pattern (CuKα) further comprises significant diffraction peaks at about 16.5, 20.1 and 25.0°2θ.

4. The polymorphic form of claim 2, wherein the X-ray diffraction pattern (CuKα) is substantially as shown in FIG. 7.

5. The polymorphic form of claim 2, wherein said Form B further having a differential scanning calorimetry (DSC) curve comprising a first and a second endotherms and an exotherm, wherein said first endotherm is centered at about 211° C., said second endotherm is forked and having peaks centered at about 331° C. and at about 338° C., and said exotherm is centered at about 245° C.

6. The polymorphic form of claim 2, wherein said Form B further having substantially a differential scanning calorimetry (DSC) curve as shown in FIG. 8.

7. The polymorphic form of claim 2, wherein said Form B is prepared by treating Compound 1 with DMA.

8. The polymorphic form of claim 1, wherein the polymorphic form is Form C which is an anhydrate having an X-ray powder diffraction pattern (CuKα) comprising significant diffraction peaks at about 17.1, 19.8 and 26.4°2θ

9. The polymorphic form of claim 8, wherein the X-ray powder diffraction pattern (CuKα) further comprises significant diffraction peaks at about 17.7 and 22.0°2θ.

10. The polymorphic form of claim 8, wherein the X-ray diffraction pattern (CuKα) is substantially as shown in FIG. 11.

11. The polymorphic form of claim 8, wherein Form C further having a differential scanning calorimetry (DSC) curve comprising an endotherm which onset at about 314° C.

12. The polymorphic form of claim 11, wherein the endotherm is centered at about 335° C.

13. The polymorphic form of claim 8, wherein said Form C having substantially a differential scanning calorimetry (DSC) curve as shown in FIG. 12.

14. The polymorphic form of claim 8, wherein said Form C is prepared by drying Compound 1.

15. The polymorphic form of claim 8, wherein said Form C is prepared by dissolving Compound 1 in an anhydrous solvent.

16. The polymorphic form of claim 1, wherein the polymorphic form is Form D which is an anhydrate having an X-ray powder diffraction pattern (CuKα) comprising significant diffraction peaks at about 7.8, 17.6, and 20.9°2θ

17. The polymorphic form of claim 16, wherein the X-ray powder diffraction pattern (CuKα) further comprises significant diffraction peaks at about 5.9 and 25.2°2θ

18. The polymorphic form of claim 16, wherein the X-ray diffraction pattern (CuKα) is substantially as shown in FIG. 16.

19. The polymorphic form of claim 16, wherein said Form D further having a differential scanning calorimetry (DSC) curve comprising an endotherm centered at about 249° C. and an exotherm centered at about 264° C.

20. The polymorphic form of claim 16, wherein said Form D further having substantially a differential scanning calorimetry (DSC) curve as shown in FIG. 17.

21. The polymorphic form of claim 16, wherein said Form D is prepared by:
dissolving Compound 1 in DMA; and
adding an antisolvent to the Compound 1 and DMA solution, wherein the antisolvent is methyl tert-butylether (MTBE).

22. The polymorphic form of claim 1, wherein said polymorphic form is Form E which is an N-methylpyrrolidinone (NMP) solvate having an X-ray powder diffraction pattern (CuKα) comprising significant diffraction peaks at about 17.0, 19.6 and 20.2°2θ.

23. The polymorphic form of claim 22, wherein the X-ray powder diffraction pattern (CuKα) further comprises significant diffraction peaks at about 13.9, 25.1 and 26.2°2θ.

24. The polymorphic form of claim 22, wherein the X-ray diffraction pattern (CuKα) is substantially as shown in FIG. 20.

25. The polymorphic form of claim 22, wherein said Form E further having a differential scanning calorimetry (DSC) curve comprising a first and a second endotherm and an exotherm, wherein said first endotherm centered at about 220° C., said second endotherm centered at about 336° C., and said exotherm centered at about 228° C.

26. The polymorphic form of claim 22, wherein said Form E further having substantially a differential scanning calorimetry (DSC) curve as shown in FIG. 21.

27. The polymorphic form of claim 22, wherein said Form E is prepared by treating Compound 1 with NMP.

28. The polymorphic form of claim 1, wherein the polymorphic form is Form F which is a desolvate having an X-ray powder diffraction pattern (CuKα) comprising significant diffraction peaks at about 7.0, 17.2, and 25.9°2θ.

29. The polymorphic form of claim 28, wherein the X-ray powder diffraction pattern (CuKα) further comprises significant diffraction peaks at about 5.2, 10.3 and 20.2°2θ.

30. The polymorphic form of claim 28, wherein the X-ray diffraction pattern (CuKα) is substantially as shown in FIG. 24.

31. The polymorphic form of claim 28, wherein said Form F further having a differential scanning calorimetry (DSC) curve comprising an endotherm which onset about 304° C.

32. The polymorphic form of claim 31, wherein the endotherm is centered at about 328° C.

33. The polymorphic form of claim 28, wherein said Form F further having substantially a differential scanning calorimetry (DSC) curve as shown in FIG. 25.

34. The polymorphic form of claim 28, wherein said Form F is prepared by treating Compound 1 with DMA or DMF, and heating the treated Compound 1.

35. The polymorphic form of claim 1, wherein the polymorphic form is Form G which is a dimethylformamide (DMF) solvate having an X-ray powder diffraction pattern (CuKα) comprising significant diffraction peaks at about 5.5, 10.9 and 22.0°2θ.

36. The polymorphic form of claim 35, wherein the X-ray powder diffraction pattern (CuKα) further comprises significant diffraction peaks at about 16.5, 18.4 and 19.5°2θ.

37. The polymorphic form of claim 35, wherein the X-ray diffraction pattern (CuKα) is substantially as shown in FIG. 27.

38. The polymorphic form of claim 35, wherein Form G further having a differential scanning calorimetry (DSC) curve comprising a broad endotherm at approximately 201° C. and a second endotherm which onsets at approximately 314° C.

39. The polymorphic form of claim 38, wherein the second endotherm is centered at about 336° C.

40. The polymorphic form of claim 35, wherein Form G further having substantially a differential scanning calorimetry (DSC) curve as shown in FIG. 28.

41. The polymorphic form of claim 35, wherein Form G is prepared by treating Compound 1 with DMF.

42. The polymorphic form of claim 1, wherein the polymorphic form is Form I which is a tetrahydrofuran (THF) solvate having an X-ray powder diffraction pattern (CuKα) comprising significant diffraction peaks at about 7.0, 16.7 and 17.4 degrees 2-theta (°2θ).

43. The polymorphic form of claim 42, wherein the X-ray powder diffraction pattern (CuKα) further comprises significant diffraction peaks at about 19.6, 20.2 and 24.6°2θ.

44. The polymorphic form of claim 42, wherein the X-ray diffraction pattern (CuKα) is substantially as shown in FIG. 31.

45. The polymorphic form of claim 42, wherein said Form I further having a differential scanning calorimetry (DSC) curve comprising a first endotherm centered at about 206° C., an exotherm centered at about 242° C., and a second endotherm onset at about 314° C. and centered at about 336° C.

46. The polymorphic form of claim 42, wherein said Form I further having substantially a differential scanning calorimetry (DSC) curve as shown in FIG. 32.

47. The polymorphic form of claim 42, wherein said Form I is prepared by treating Compound 1 with THF.

48. The polymorphic form of claim 1, wherein the polymorphic form is Form J which is an anhydrate having an X-ray powder diffraction pattern (CuKα) comprising significant diffraction peaks at about 4.9, 17.5 and 20.0 degrees 2-theta (°2θ).

49. The polymorphic form of claim 48, wherein the X-ray powder diffraction pattern (CuKα) further comprises significant diffraction peaks at about 9.2, 22.1 and 25.2°2θ.

50. The polymorphic form of claim 48, wherein the X-ray diffraction pattern (CuKα) is substantially as shown in FIG. 35.

51. The polymorphic form of claim 48, wherein said Form J further having a differential scanning calorimetry (DSC) curve comprising a first endotherm centered at about 219° C., a forked exotherm having peaks centered at about 223° C. and 236° C., and a forked endotherm which onsets at about 302° C.

52. The polymorphic form of claim 51, wherein the forked endotherm having peaks centered at approximately 323° C., 328° C. and 338° C.

53. The polymorphic form of claim 48, wherein said Form J further having substantially a differential scanning calorimetry (DSC) curve as shown in FIG. 36.

54. The polymorphic form of claim 48, wherein said Form J is prepared by treating Compound 1 with isopropyl alcohol.

55. The polymorphic form of claim 1, wherein the polymorphic form is Form K which is an anhydrate having an X-ray powder diffraction pattern (CuKα) comprising significant diffraction peaks at about 5.3, 8.5 and 10.5°2θ.

56. The polymorphic form of claim 55, wherein the X-ray powder diffraction pattern (CuKα) further comprises significant diffraction peaks at about 13.3, 18.6 and 21.3°2θ.

57. The polymorphic form of claim 55, wherein the X-ray diffraction pattern (CuKα) is substantially as shown in FIG. 39.

58. The polymorphic form of claim 55, wherein said Form K further having a differential scanning calorimetry (DSC) curve comprising an endotherm onset at about 306° C.

59. The polymorphic form of claim 58, wherein the endotherm is centered at about 322° C.

60. The polymorphic form of claim 55, wherein said Form K further having substantially a differential scanning calorimetry (DSC) curve as shown in FIG. 40.

61. The polymorphic form of claim 55, wherein said Form K is prepared by:
dissolving Compound 1 in EtOH; and
adding THF to the solution.

62. The polymorphic form of claim 1, wherein said polymorphic form is Form L which is a channel hydrate having an X-ray powder diffraction pattern (CuKα) comprising significant diffraction peaks at about 5.2, 10.4 and 20.7 degrees 2-theta (°2θ).

63. The polymorphic form of claim 62, wherein the X-ray powder diffraction pattern (CuKα) further comprises significant diffraction peaks at about 15.5, 16.9 and 24.4°2θ.

64. The polymorphic form of claim 62, wherein the X-ray diffraction pattern (CuKα) is substantially as shown in FIG. 43.

65. The polymorphic form of claim 62, wherein said Form L having a differential scanning calorimetry (DSC) curve comprising an endotherm which onsets at about 303° C.

66. The polymorphic form of claim 65, wherein the endotherm is centered at about 333° C.

67. The polymorphic form of claim 62, wherein said Form L having substantially a differential scanning calorimetry (DSC) curve as shown in FIG. 44.

68. The polymorphic form of claim 62, wherein said Form L is prepared by
dissolving Compound 1 in methanol; and
adding an antisolvent to Compound 1 dissolved in the solvent, wherein the antisolvent is selected from the group consisting of methyl tert-butylether, isopropyl acetate and heptane.

69. The polymorphic form of Compound 1, wherein the polymorphic form is Form M having an X-ray powder diffraction pattern (CuKα) comprising significant diffraction peaks at about 5.1, 8.2 and 10.2 degrees 2-theta (°2θ).

70. The polymorphic form of claim 69, wherein the X-ray powder diffraction pattern (CuKα) further comprises significant diffraction peaks at about 18.1 and 20.6°2θ.

71. The polymorphic form of claim 69, wherein the X-ray diffraction pattern (CuKα) is substantially as shown in FIG. 48.

72. The polymorphic form of claim 69, wherein Form M further having a differential scanning calorimetry (DSC) curve comprising an endotherm centered at about 332° C.

73. The polymorphic form of claim 69, wherein said Form M further having substantially a differential scanning calorimetry (DSC) curve as shown in FIG. 49.

74. The polymorphic form of claim 69, wherein said Form M is prepared by treating Compound 1 with water.

75. The polymorphic form of Compound 1, wherein the polymorphic form is Form N having an X-ray powder diffraction pattern (CuKα) comprising significant diffraction peaks at about 5.2, 8.4 and 10.3°2θ.

76. The polymorphic form of claim 75, wherein the X-ray powder diffraction pattern (CuKα) further comprises significant diffraction peaks at about 18.6, 20.0 and 21.0°2θ.

77. The polymorphic form of claim 75, wherein the X-ray diffraction pattern (CuKα) is substantially as shown in FIG. 52.

78. The polymorphic form of claim 75, wherein said Form N further having a differential scanning calorimetry (DSC) curve comprising an endotherm centered at about 333° C.

79. The polymorphic form of claim 75, wherein said Form N further having substantially a differential scanning calorimetry (DSC) curve as shown in FIG. 53.

80. The polymorphic form of claim 75, wherein said Form N is prepared by treating Compound 1 with water.

81. The polymorphic form of claim 1, wherein said polymorphic form is Form O which is a dehydrate having an X-ray powder diffraction pattern (CuKα) comprising significant diffraction peaks at about 6.3, 12.6 and 25.3°2θ.

82. The polymorphic form of claim 81, wherein the X-ray powder diffraction pattern (CuKα) further comprises significant diffraction peaks at about 10.5 and 21.0°2θ.

83. The polymorphic form of claim 81, wherein the X-ray diffraction pattern (CuKα) is substantially as shown in FIG. 56.

84. The polymorphic form of claim 81, wherein said Form O further having a differential scanning calorimetry (DSC) curve comprising an endotherm centered at about 327° C.

85. The polymorphic form of claim 81, wherein said Form O further having substantially a differential scanning calorimetry (DSC) curve as shown in FIG. 57.

86. The polymorphic form of claim 81, wherein said Form O is prepared by
treating Compound 1 with water; and
heating the treated Compound 1.

87. The polymorphic form of claim 1, wherein the polymorphic form is Form P which having an X-ray powder diffraction pattern (CuKα) comprising significant diffraction peaks at about 5.0, 9.4 and 10.0 degrees 2-theta (°2θ).

88. The polymorphic form of claim 87, wherein the X-ray powder diffraction pattern (CuKα) further comprises significant diffraction peaks at about 17.2 and 25.7°2θ.

89. The polymorphic form of claim 87, wherein the X-ray diffraction pattern (CuKα) is substantially as shown in FIG. 59.

90. A pharmaceutical composition comprising, as an active ingredient, Compound 1 of the formula: 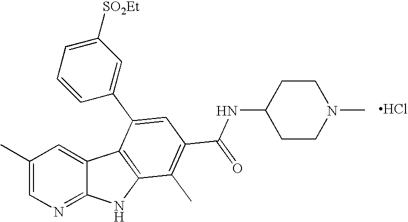
and a pharmaceutically acceptable carrier, wherein at least a portion of Compound 1 is present as a polymorphic form selected from the group consisting of Form B, Form C, Form D, Form E, Form F, Form G, Form I, Form J, Form K, Form L, Form M, Form N, Form O and Form P.

91. The pharmaceutical composition of claim 82, wherein the portion of polymorphic form is between about 0.1% to about 100%.

92. The pharmaceutical composition of claim 82, wherein said portion is greater than 1%.

93. The pharmaceutical composition of claim 82, wherein said portion is greater than 10%.

94. The pharmaceutical composition of claim 82, wherein said portion is greater than 90%.

95. A therapeutic method comprising:
administering Compound 1, wherein at least a portion of Compound 1 is present as a polymorphic form selected from the group consisting of Form B, Form C, Form D, Form E, Form F, Form G, Form I, Form J, Form K, Form L, Form M, Form N, Form O and Form P

96. A method of treating a disease state for which a kinase possesses activity that contributes to the pathology and/or symptomology of the disease state, the method comprising:
administering Compound 1 to a subject in need thereof, wherein at least a portion of Compound 1 is present as a polymorphic form selected from the group consisting of Form B, Form C, Form D, Form E, Form F, Form G, Form I, Form J, Form K, Form L, Form M, Form N, Form O and Form P.

97. A method of treating cancer comprising administering a therapeutically effective amount of Compound 1 to a mammalian species in need thereof, wherein at least a portion of Compound 1 is present as a polymorphic form selected from the group consisting of Form B, Form C, Form D, Form E, Form F, Form G, Form I, Form J, Form K, Form L, Form M, Form N, Form O and Form P.

98. The method according to claim 97, wherein the cancer is selected from the group consisting of squamous cell carcinoma, astrocytoma, Kaposi's sarcoma, glioblastoma, small-cell lung cancer, non small-cell lung cancers, bladder cancer, head and neck cancer, melanoma, ovarian cancer, prostate cancer, breast cancer, glioma, colorectal cancer, genitourinary cancer, gastrointestinal cancer, thyroid cancer, skin cancer, kidney cancer, rectal cancer, colonic cancer, cervical cancer, mesothelioma, pancreatic cancer, liver cancer, uterus cancer, cerebral tumor cancer, urinary bladder cancer and blood cancers including multiple myeloma, chronic myelogenous leukemia and acute lymphocytic leukemia.

99. A method for preventing or treating dementia related diseases, Alzheimer's Disease and conditions associated with kinases, comprising administration to a mammalian species in need thereof of a therapeutically effective amount of Compound 1, wherein at least a portion of Compound 1 is present as a polymorphic form selected from the group consisting of Form B, Form C, Form D, Form E, Form F, Form G, Form I, Form J, Form K, Form L, Form M, Form N, Form O and Form P.

100. The method according to claim 99, wherein the dementia related diseases are selected from the group consisting of Frontotemporal dementia Parkinson's Type, Parkinson dementia complex of Guam, HIV dementia, diseases with associated neurofibrillar tangle pathologies, predemented states, vascular dementia, dementia with Lewy bodies, Frontotemporal dementia and dementia pugilistica.

101. A method for treating arthritis comprising administration to a mammal in need thereof a therapeutically effective amount of Compound 1, wherein at least a portion of Compound 1 is present as a polymorphic form selected from the group consisting of Form B, Form C, Form D, Form E, Form F, Form G, Form I, Form J, Form K, Form L, Form M, Form N, Form O and Form P.

102. A method of inhibiting cell proliferation in a patient comprising administering to the patient a therapeutically effective amount of Compound 1, wherein at least a portion of Compound 1 is present as a polymorphic form selected from the group consisting of Form B, Form C, Form D, Form E, Form F, Form G, Form I, Form J, Form K, Form L, Form M, Form N, Form O and Form P.
